(19) United States
(12) Patent Application Publication
(10) Pub. No.: US 2014/0170157 A1
(43) Pub. Date: Jun. 19, 2014

(54) METHOD OF SELECTING THERAPEUTIC INDICATIONS

(71) Applicants: Pankaj Agarwal, King of Prussia, PA (US); Lon R. Cardon, King of Prussia, PA (US); Vincent Eugene Mooser, Lausanne (CH); Philippe Sanseau, Stevenage (GB)

(72) Inventors: Pankaj Agarwal, King of Prussia, PA (US); Lon R. Cardon, King of Prussia, PA (US); Vincent Eugene Mooser, Lausanne (CH); Philippe Sanseau, Stevenage (GB)

(73) Assignee: GLAXOSMITHKLINE INTELLECTUAL PROPERTY (NO.2) LIMITED, Brentford, Middlesex (GB)

(21) Appl. No.: 14/126,125

(22) PCT Filed: Jun. 15, 2012
(86) PCT No.: PCT/US2012/042601

§ 371 Date: Dec. 13, 2013

Related U.S. Application Data
(60) Provisional application No. 61/497,302, filed on Jun. 15, 2011.
(60) Provisional application No. 61/514,623, filed on Aug. 3, 2011.

(51) Int. Cl. C12Q 1/68; A61K 31/215; A61K 31/7072; A61K 31/4152

(52) U.S. Cl. CPC C12Q 1/6883; A61K 31/4152; A61K 31/215; A61K 31/7072
USPC 424/153.1; 424/172.1; 435/6.11; 514/404; 514/510; 514/50

(57) ABSTRACT

Methods of treatment for Crohn's disease and multiple sclerosis are disclosed. Also disclosed are methods for repositioning a pharmaceutical by selecting at least one target gene, gene product, or loci associated with the treatment of at least one first disease, trait, or phenotype by the pharmaceutical, identifying at least one second disease, trait, or phenotype associated with the least one target gene, gene product, or loci using genome-wide associated studies, and selecting at least one identified second disease, trait and/or phenotype based on step for treatment with the pharmaceutical.

### FIELD OF THE INVENTION

The invention relates to methods for selecting a therapeutic indication for a pharmaceutical as well as methods of treating various disease and disorders with a pharmaceutical.

### BACKGROUND OF THE INVENTION

A genome-wide association study (GWAS) is an approach that involves rapidly scanning markers across the complete sets of DNA, or genomes, of many people to find genetic variations associated with a particular disease. In theory, once new genetic associations are identified, researchers can use the information to develop medicines to treat and prevent the disease. However, the promise that genome-wide associated studies (GWAS) studies will lead to novel therapeutics has not yet materialized partly because of the 10+ year lag time between identifying a new drug target discovering and developing novel medicines to the target.

Thus, methods are needed for translating GWAS results to identify new or unsuspected indications for existing pharmaceuticals. Additionally, methods are needed for validating or invalidating a first therapeutic indication of a pharmaceutical as well as selecting at least a therapeutic agent for treatment or prevention of a disease and/or disorder.

### BRIEF DESCRIPTION OF THE DRAWINGS

FIG. 1: Analysis pipeline: 991 GWAS associated genes were selected from the GWAS catalog after two filtering steps (see material and methods for details). These genes were evaluated as potential drug targets for small molecules and bipharmaceuticals. 155 of these 991 genes were also targeted by drugs currently in pharmaceutical pipelines based on the Pharmaprojects database that has 1,089 genes targeted by drugs. When the disease for the drug was identical to the GWAS disease trait for the gene it increased the confidence that the drug was for an appropriate disease indication. When it was different it could be a potential opportunity to use the drug for the disease trait the GWAS. 63 individual genes map to 52 different GWAS traits and drugs with the same or closely related indication to the GWAS traits (considered as matches). 92 individual genes map to 51 GWAS traits and drugs with indications different from the GWAS traits (considered mismatches or potential drug repositioning opportunities). Some genes are in both lists as they have multiple GWAS phenotypes that resulted in both a match to an indication and also a novel indication.

### SUMMARY OF THE INVENTION

Methods are provided for treating Crohn's disease in a human in need thereof, comprising administering denosumab to said human.

Methods are provided for treating Crohn's disease in a human in need thereof, comprising administering to said human at least one compound selected from the group consisting of: an inhibitor and/or antagonist of tumor necrosis factor ligand, a T-cell co-stimulatory ligand, an IL-18 receptor antagonist and/or inhibitor, inducer of IL27 expression, anti-IL2 receptor mAb, chemokine (C—C motif) ligand 2 inhibitor and/or antagonist, estrogen related receptor alpha binding agent, galactosylceramidase, anti-Intercellular adhesion molecule 3 (ICAM3) mAb, anti-ICOS mAb, IL-23 receptor inhibitor and/or antagonist, Janus kinase 2 inhibitor, leucine-rich repeat kinase 2 inhibitor, mucin 1, cell surface associated inhibitor, signal transducer and activator of transcription 3 (acute-phase response factor) inhibitor, and tyrosine kinase 2 inhibitor.

Methods are provided for treating multiple sclerosis in a human in need thereof, comprising administering a STAT3 inhibitor to said human.

Methods are provided for repositioning a pharmaceutical, comprising the steps of:
a. selecting at least one target gene or gene product associated with the treatment of at least one first disease, trait and/or phenotype by said pharmaceutical,
b. identifying at least one second disease, trait and/or phenotype for at least one target gene of pharmaceutical mentioned in (a) using genome-wide associated studies, and
c. selecting at least one second disease, trait and/or phenotype based on step (b) for treatment with said pharmaceutical compound in step (a).

Methods are also provided for validating or invalidating a first therapeutic indication of a pharmaceutical comprising matching all GWAS associated diseases, traits and/or phenotypes of at least one target gene associated with said first therapeutic indication and determining if at least one GWAS associated disease, trait and/or phenotype of said target gene is associated with said first therapeutic indication.

Methods are also provided for selecting a therapeutic agent for treatment or prevention of a disease comprising the steps of:
a. selecting at least one target gene or gene product associated with at least one disease, trait and/or phenotype by at least one genome-wide associated study;
b. optionally determining if said target gene or gene product is small molecule druggable, secreted protein, antibody accessible, or modifiable by some other modality including anti-sense oligos, gene therapy, aptamers;
c. determining if said target is under expressed or over expressed for a particular disease, trait and/or phenotype;
d. selecting a therapeutic agent that is an agonist to said target gene or gene product if said gene is under reduced functionality in said disease and/or trait of step (a) and selecting a therapeutic agent that is an antagonist to said target gene or gene product if said gene is under increased functionality in a particular disease and/or trait.

### DETAILED DESCRIPTION

With the completion of the Human Genome Project in 2003 and the International HapMap Project in 2005, researchers now have a set of research tools that make it possible to find the genetic contributions to common diseases. The tools include computerized databases that contain the reference human genome sequence, a map of human genetic variation and a set of new technologies that can quickly and accurately analyze whole-genome samples for genetic variations that contribute to the onset of a disease.

To carry out a genome-wide association study, researchers obtain DNA from participants in one of two groups: people with a selected disease, trait and/or phenotype and similar people without that disease trait and/or phenotype. Each person's complete set of DNA, or genome, is surveyed for strategically selected markers of genetic variation, which are called single nucleotide polymorphisms, or SNPs. If certain genetic variations are found to be significantly more frequent in people with the disease compared to people without disease, the variations are said to be “associated” with the disease. The associated genetic variations can serve as powerful pointers to the region of the human genome where the disease-causing problem resides.

Associated variants may either directly or indirectly cause selected disease, trait and/or phenotype. Therefore, further genotyping of DNA base pairs in a particular region of the genome may be necessary to identify the exact genetic change involved in the selected disease, trait and/or phenotype.

As used herein, “genotyping” a subject (or DNA or other biological sample) for a polymorphic allele of a gene(s) means detecting which allelic or polymorphic form(s) of the gene(s) or gene expression products (e.g., hnRNA, mRNA or protein) are present or absent in a subject (or a sample). Related RNA or protein expressed from such gene may also be used to detect polymorphic variation. As is well known in the art, an individual may be heterozygous or homozygous for a particular allele. More than two allelic forms may exist, thus, there may be more than three possible genotypes. For purposes of the present invention, “genotyping” includes the determination of HLA alleles using suitable serologic techniques, as are known in the art. As used herein, an allele may be ‘detected’ when other possible allelic variants have been ruled out; e.g., where a specified nucleic acid position is found to be neither adenine (A), thymine (T) or cytosine (C), it can be concluded that guanine (G) is present at that position (i.e., G is ‘detected’ or ‘diagnosed’ in a subject). Sequence variations may be detected directly (by, e.g, sequencing) or indirectly (e.g., by restriction fragment length polymorphism analysis, or detection of the hybridization of a probe of known sequence, or reference strand conformation polymorphism), or by using other known methods.

As used herein, a “genetic subset” of a population consists of those members of the population having a particular genotype. In the case of a biallelic polymorphism, a population can potentially be divided into three subsets: homozygous for allele 1 (1,1), heterozygous (1,2), and homozygous for allele 2 (2,2). A ‘population’ of subjects may be defined using various criteria, e.g., individuals being treated with lapatinib or individuals with cancer.

As used herein, a subject that is “predisposed to” or “at increased risk of” a particular disease, trait and/or phenotypic response based on genotyping will be more likely to display that phenotype than an individual with a different genotype at the target polymorphic locus (or loci). Where the phenotypic response is based on a multi-allelic polymorphism, or on the genotyping of more than one gene, the relative risk may differ among the multiple possible genotypes.

An allele refers to one specific form of a genetic sequence (such as a gene) within a cell, a sample, an individual or within a population, the specific form differing from other forms of the same gene in the sequence of at least one, and frequently more than one, variant sites within the sequence of the gene. The sequences at these variant sites that differ between different alleles are termed “variants”, “polymorphisms”, or “mutations.” In general, polymorphism is used to refer to variants that have a frequency of at least 1% in a population, while the term mutation is generally used for variants that occur at a frequency of less than 1% in a population. In diploid organisms such as humans, at each autosomal specific chromosomal location or “locus” an individual possesses two alleles, a first inherited from one parent and a second inherited from the other parent, for example one from the mother and one from the father. An individual is “heterozygous” at a locus if it has two different alleles at the locus. An individual is “homozygous” at a locus if it has two identical alleles at that locus.

A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wild type form. The most frequent allele may also be referred to as the major allele and the less frequent allele as the minor allele. Diploid organisms may be homozygous or heterozygous for allelic forms. A diallelic polymorphism has two forms. A triallelic polymorphism has three forms. A polymorphism between two nucleic acids can occur naturally, or be caused by exposure to or contact with chemicals, enzymes, or other agents, or exposure to agents that cause damage to nucleic acids, for example, ultraviolet radiation, mutagens or carcinogens.

Single nucleotide polymorphisms (SNPs) are positions at which two alternative bases occur at appreciable frequency (>1%) in the human population, and are the most common type of human genetic variation. Approximately 90% of all polymorphisms in the human genome are SNPs. SNPs are single base positions in DNA at which different alleles, or alternative nucleotides, exist in a population. An individual may be homozygous or heterozygous for an allele at each SNP position. A SNP can, in some instances, be referred to as a “cSNP” to denote that the nucleotide sequence containing the SNP is an amino acid coding sequence. As used herein, references to SNPs and SNP genotypes include individual SNPs and/or haplotypes, which are groups of SNPs that are generally inherited together. Haplotypes can have stronger correlations with diseases or other phenotypic effects compared with individual SNPs, and therefore may provide increased diagnostic accuracy in some cases (Stephens et al. Science 293, 489-493, 20 Jul. 2001).

Causative SNPs are those SNPs that produce alterations in gene expression or in the expression, structure, and/or function of a gene product, and therefore are most predictive of a possible clinical phenotype. One such class includes SNPs falling within regions of genes encoding a polypeptide product, i.e. cSNPs. These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Causative SNPs do not necessarily have to occur in coding regions; causative SNPs can occur in, for example, any genetic region that can ultimately affect the expression, structure, and/or activity of the protein encoded by a nucleic acid. Such genetic regions include, for example, those involved in transcription, such as SNPs in transcription factor binding domains, SNPs in promoter regions, in areas involved in transcript processing, such as SNPs at intron-exon boundaries that may cause defective splicing, or SNPs in mRNA processing signal sequences such as polyadenylation signal regions. Some SNPs that are not causative SNPs nevertheless are in close association with, and therefore segregate with, a disease-causing sequence. In this situation, the presence of a SNP correlates with the presence of, or predisposition to, or an increased risk in developing the disease. These SNPs, although not causative, are nonetheless also useful for diagnostics, disease predisposition screening, and other uses.

An association study of a SNP and a specific disorder or a predisposition to a safety event involves determining the presence or frequency of the SNP allele in biological samples from individuals with the disorder or predisposition to a safety event of interest and comparing the information to that of controls (i.e., individuals who do not have the disorder or experience the same safety event).

A SNP may be screened in diseased tissue samples or any biological sample obtained from an individual, and compared to control samples, and selected for its increased (or decreased) occurrence in a specific pathological condition. Once a statistically significant association is established between one or more SNP(s) and a pathological condition (or other phenotype) of interest, then the region around the SNP can optionally be thoroughly screened to identify the causative genetic locus/sequence(s) (e.g., causative SNP/mutation, gene, regulatory region, etc.) that influences the pathological condition or phenotype.

Clinical trials have shown that patient response to treatment with pharmaceuticals is often heterogeneous. There is a continuing need to improve pharmaceutical agent design and therapy. In that regard, SNPs can be used to identify patients most suited to therapy with particular pharmaceutical agents (this is often termed “pharmacogenomics”). Similarly, SNPs can be used to exclude patients from certain treatment due to the patient's increased likelihood of developing toxic side effects or their likelihood of not responding to the treatment. Pharmacogenomics can also be used in pharmaceutical research to assist the drug development and selection process. (Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 3).

Several techniques for the detection of mutations have evolved based on the principal of hybridization analysis. For example, in the primer extension assay, the DNA region spanning the nucleotide of interest is amplified by PCR, or any other suitable amplification technique. After amplification, a primer is hybridized to a target nucleic acid sequence, wherein the last nucleotide of the 3′ end of the primer anneals immediately 5′ to the nucleotide position on the target sequence that is to be analyzed. The annealed primer is extended by a single, labelled nucleotide triphosphate. The incorporated nucleotide is then detected.

The sequence of any nucleic acid including a gene or PCR product or a fragment or portion thereof may be sequenced by any method known in the art (e.g., chemical sequencing or enzymatic sequencing). “Chemical sequencing” of DNA may denote methods such as that of Maxam and Gilbert (1977) (Proc. Natl. Acad. Sci. USA 74:560), in which DNA is randomly cleaved using individual base-specific reactions. “Enzymatic sequencing” of DNA may denote methods such as that of Sanger (Sanger, et al., (1977) Proc. Natl. Acad. Sci. USA 74:5463).

Conventional molecular biology, microbiology, and recombinant DNA techniques including sequencing techniques are well known among those skilled in the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994

The Peptide Nucleic Acid (PNA) affinity assay is a derivative of traditional hybridization assays (Nielsen et al., Science 254:1497-1500 (1991); Egholm et al., J. Am. Chem. Soc. 114:1895-1897 (1992); James et al., Protein Science 3:1347-1350 (1994)). PNAs are structural DNA mimics that follow Watson-Crick base pairing rules, and are used in standard DNA hybridization assays. PNAs display greater specificity in hybridization assays because a PNA/DNA mismatch is more destabilizing than a DNA/DNA mismatch and complementary PNA/DNA strands form stronger bonds than complementary DNA/DNA strands.

DNA microarrays have been developed to detect genetic variations and polymorphisms (Taton et al., Science 289:1757-60, 2000; Lockhart et al., Nature 405:827-836 (2000); Gerhold et al., Trends in Biochemical Sciences 24:168-73 (1999); Wallace, R. W., Molecular Medicine Today 3:384-89 (1997); Blanchard and Hood, Nature Biotechnology 149:1649 (1996)). DNA microarrays are fabricated by high-speed robotics, on glass or nylon substrates, and contain DNA fragments with known identities (“the probe”). The microarrays are used for matching known and unknown DNA fragments (“the target”) based on traditional base-pairing rules.

The Protein Truncation Test (PTT) is also commonly used to detect genetic polymorphisms (Roest et al., Human Molecular Genetics 2:1719-1721, (1993); Van Der Luit et al., Genomics 20:1-4 (1994); Hogervorst et al., Nature Genetics 10: 208-212 (1995)). Typically, in the PTT, the gene of interest is PCR amplified, subjected to in vitro transcription/translation, purified, and analyzed by polyacrylamide gel electrophoresis.

“Genetic testing” (also called genetic screening) as used herein refers to the testing of a biological sample from a subject to determine the subject's genotype; and may be utilized to determine if the subject's genotype comprises alleles that either cause, or increase susceptibility to, a particular phenotype (or that are in linkage disequilibrium with allele(s) causing or increasing susceptibility to that phenotype).

“Linkage disequilibrium” refers to the tendency of specific alleles at different genomic locations to occur together more frequently than would be expected by chance. Alleles at given loci are in complete equilibrium if the frequency of any particular set of alleles (or haplotype) is the product of their individual population frequencies A commonly used measure of linkage disequilibrium is r:

$$r = \frac{{\hat{Δ}}_{AB}}{\sqrt{( {\tilde{π}}_{A} + {\hat{D}}_{A} ) ( {\tilde{π}}_{B} + {\hat{D}}_{B} )}}$$

$$where$$

$${\tilde{π}}_{A} = {\tilde{p}}_{A} ( \dot{1} - {\tilde{p}}_{A} ) , {\tilde{π}}_{B} = {\tilde{p}}_{B} ( 1 - {\tilde{p}}_{B} ) , {\hat{D}}_{A} = {\tilde{P}}_{AA} - {\tilde{p}}_{A}^{2} , {\hat{D}}_{B} = {\tilde{P}}_{BB} - {\tilde{p}}_{B}^{2}$$

$${\hat{Δ}}_{AB} = \frac{1}{n} {n}_{AB} - 2 {\tilde{p}}_{A} {\tilde{p}}_{B}$$

nr² has an approximate chi square distribution with 1 degree freedom for biallelic markers. Loci exhibiting an r such that nr² is greater than 3.84, corresponding to a significant chi-squared statistic at the 0.05 level, are considered to be in linkage disequilibrium (BS Weir 1996 Genetic Data Analysis II Sinauer Associates, Sunderland, Md.).

Alternatively, a normalized measure of linkage disequilibrium can be defined as:

$${D}_{AB}^{′} = \{ \begin{matrix}\frac{{D}_{AB}}{min ( {p}_{A} {p}_{B} , {p}_{a} {p}_{b} )} , & {D}_{AB} < 0 \\ \frac{{D}_{AB}}{min ( {p}_{A} {p}_{b} , {p}_{a} {p}_{B} )} , & {D}_{AB} > 0\end{matrix}$$

The value of the D′ has a range of −1.0 to 1.0. When statistically significant absolute D′ value for two markers is not less than 0.3 they are considered to be in linkage disequilibrium.

Polymorphic alleles may be detected by determining the DNA polynucleotide sequence, or by detecting the corresponding sequence in RNA transcripts from the polymorphic gene, or where the nucleic acid polymorphism results in a change in an encoded protein by detecting such amino acid sequence changes in encoded proteins; using any suitable technique as is known in the art. Polynucleotides utilized for typing are typically genomic DNA, or a polynucleotide fragment derived from a genomic polynucleotide sequence, such as in a library made using genomic material from the individual (e.g. a cDNA library). The polymorphism may be detected in a method that comprises contacting a polynucleotide or protein sample from an individual with a specific binding agent for the polymorphism and determining whether the agent binds to the polynucleotide or protein, where the binding indicates that the polymorphism is present. The binding agent may also bind to flanking nucleotides and amino acids on one or both sides of the polymorphism, for example at least 2, 5, 10, 15 or more flanking nucleotide or amino acids in total or on each side. In the case where the presence of the polymorphism is being determined in a polynucleotide it may be detected in the double stranded form, but is typically detected in the single stranded form.

The binding agent may be a polynucleotide (single or double stranded) typically with a length of at least 10 nucleotides, for example at least 15, 20, 30, or more nucleotides. A polynucleotide agent which is used in the method will generally bind to the polymorphism of interest, and the flanking sequence, in a sequence specific manner (e.g. hybridize in accordance with Watson-Crick base pairing) and thus typically has a sequence which is fully or partially complementary to the sequence of the polymorphism and flanking region. The binding agent may be a molecule that is structurally similar to polynucleotides that comprises units (such as purine or pyrimidine analogs, peptide nucleic acids, or RNA derivatives such as locked nucleic acids (LNA)) able to participate in Watson-Crick base pairing. The agent may be a protein, typically with a length of at least 10 amino acids, such as at least 20, 30, 50, or 100 or more amino acids. The agent may be an antibody (including a fragment of such an antibody that is capable of binding the polymorphism).

A binding agent can be used as a probe. The probe may be labelled or may be capable of being labelled indirectly. The detection of the label may be used to detect the presence of the probe on (bound to) the polynucleotide or protein of the individual. The binding of the probe to the polynucleotide or protein may be used to immobilize either the probe or the polynucleotide or protein (and, thus, to separate it from one composition or solution).

Polynucleotides or proteins of the individual can also be immobilized on a solid support and then contacted with the probe. The presence of the probe immobilized to the solid support (via its binding to the polymorphism) is then detected, either directly by detecting a label on the probe or indirectly by contacting the probe with a moiety that binds the probe. In the case of detecting a polynucleotide polymorphism the solid support is generally made of nitrocellulose or nylon. In the case of a protein polymorphism the method may be based on an ELISA system.

Polymorphism can also be detected using a oligonucleotide ligation assay in which two oligonucleotide probes are used. These probes bind to adjacent areas on the polynucleotide which contains the polymorphism, allowing (after binding) the two probes to be ligated together by an appropriate ligase enzyme. However the two probes will only bind (in a manner which allows ligation) to a polynucleotide that contains the polymorphism, and therefore the detection of the ligated product may be used to determine the presence of the polymorphism.

Probes can also be used in a heteroduplex analysis based system to detect polymorphisms. In such a system when the probe is bound to a polynucleotide sequence containing the polymorphism, it forms a heteroduplex at the site where the polymorphism occurs (i.e. it does not form a double strand structure). Such a heteroduplex structure can be detected by the use of an enzyme that is single or double strand specific. Typically the probe is an RNA probe and the enzyme used is RNAse H that cleaves the heteroduplex region, thus, allowing the polymorphism to be detected by means of the detection of the cleavage products.

The method may be based on fluorescent chemical cleavage mismatch analysis which is described for example in PCR Methods and Applications 3:268-71 (1994) and Proc. Natl. Acad. Sci. 85:4397-4401 (1998).

Polymorphisms can also be detected using polynucleotide agents that are able to act as a primer for a PCR reaction only if it binds a polynucleotide containing the polymorphism (i.e. a sequence- or allele-specific PCR system). Thus, a PCR product will only be produced if the polymorphism is present in the polynucleotide of the individual, and the presence of the polymorphism is determined by the detection of the PCR product. Preferably the region of the primer which is complementary to the polymorphism is at or near the 3′ end the primer. The he polynucleotide agent may also bind to the wild-type sequence but will not act as a primer for a PCR reaction.

The method may be a Restriction Fragment Length Polymorphism (RFLP) based system. This method can be used if the presence of the polymorphism in the polynucleotide creates or destroys a restriction site that is recognized by a restriction enzyme. Thus, treatment of a polynucleotide that has such a polymorphism will lead to different products being produced compared to the corresponding wild-type sequence. Thus, the detection of the presence of particular restriction digest products can be used to determine the presence of the polymorphism.

The presence of the polymorphism may be determined based on the change that the presence of the polymorphism makes to the mobility of the polynucleotide or protein during gel electrophoresis. In the case of a polynucleotide single-stranded conformation polymorphism (SSCP) analysis may be used. SSCP measures the mobility of the single stranded polynucleotide on a denaturing gel compared to the corresponding wild-type polynucleotide, the detection of a difference in mobility indicating the presence of the polymorphism. Denaturing gradient gel electrophoresis (DGGE) is a similar system where the polynucleotide is electrophoresed through a gel with a denaturing gradient, a difference in mobility compared to the corresponding wild-type polynucleotide indicating the presence of the polymorphism.

The presence of the polymorphism may be determined using a fluorescent dye and quenching agent-based PCR assay such as the TAQMAN™ PCR detection system. In another method of detecting the polymorphism a polynucleotide comprising the polymorphic region is sequenced across the region which contains the polymorphism to determine the presence of the polymorphism.

Various other detection techniques suitable for use in the present methods will be apparent to those conversant with methods of detecting, identifying, and/or distinguishing polymorphisms. Such detection techniques include but are not limited to direct sequencing, use of “molecular beacons” (oligonucleotide probes that fluoresce upon hybridization, useful in real-time fluorescence PCR; see e.g., Marras et al., Genet Anal 14:151 (1999)); electrochemical detection (reduction or oxidation of DNA bases or sugars; see U.S. Pat. No. 5,871,918 to Thorp et al.); rolling circle amplification (see, e.g., Gusev et al., Am J Pathol 159:63 (2001)); Third Wave Technologies (Madison Wis.) INVADER® non-PCR based detection method (see, e.g., Lieder, Advance for Laboratory Managers, 70 (2000))

Accordingly, any suitable detection technique as is known in the art may be utilized in the present methods.

As used herein, “determining” a subject's genotype does not require that a genotyping technique be carried out where a subject has previously been genotyped and the results of the previous genetic test are available; determining a subject's genotype accordingly includes referring to previously completed genetic analyses.

As used herein “pharmaceutical” means any active ingredient capable of treating or preventing at least one disease, trait and/or phenotype. The pharmaceutical compositions of the invention are prepared using techniques and methods known to those skilled in the art. Some of the methods commonly used in the art are described in Remington's Pharmaceutical Sciences (Mack Publishing Company).

As used herein “druggable” means a characteristic that allows a compound or composition to be developed into a drug. For example, a druggable compound or composition could have at least one of the following characteristics: capable of being formulated for administration to a mammal, capable of reaching its target once administered to a mammal, and/or capable of effecting at least one target. Similarly, the term “biopharmable” refers to large molecule such as, but not limited to, proteins, antibodies, antibody fragments, domain antibodies, single chain antibodies, bispecific antibodies, and any combination or variations thereof, aptamers, fusion proteins, synthetic polypeptides, recombinant polypeptides, vaccines, DNA therapies, and/or RNAi, that can be administered to a mammal.

By the term “treating” and grammatical variations thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

As used herein “reposition” and “repositioning” and grammatical variations thereof refers to a disease, trait and/or phenotype for which a pharmaceutical may have a use beyond the first disease, trait and/or phenotype for which the pharmaceutical had identified activity.

As used herein the term “amplification” and grammatical variations thereof refers to the presence of one or more extra gene copies in a chromosome complement. In certain embodiments a gene encoding a Ras protein may be amplified in a cell. Amplification of the HER2 gene has been correlated with certain types of cancer. Amplification of the HER2 gene has been found in human salivary gland and gastric tumor-derived cell lines, gastric and colon adenocarcinomas, and mammary gland adenocarcinomas. Semba et al., Proc. Natl. Acad. Sci. USA, 82:6497-6501 (1985); Yokota et al., Oncogene, 2:283-287 (1988); Zhou et al., Cancer Res., 47:6123-6125 (1987); King et al., Science, 229:974-976 (1985); Kraus et al., EMBO J., 6:605-610 (1987); van de Vijver et al., Mol. Cell. Biol., 7:2019-2023 (1987); Yamamoto et al., Nature, 319:230-234 (1986).

As used herein “overexpressed” and “overexpression” of a protein or polypeptide and grammatical variations thereof means that a given cell produces an increased number of a certain protein relative to a normal cell of the same type. By way of example, a protein may be overexpressed by diseased cell relative to a normal cell. Additionally, a mutant protein may be overexpressed compared to wild type protein in a cell. As is understood in the art, expression levels of a polypeptide in a cell can be normalized to a housekeeping gene such as actin. In some instances, a certain polypeptide may be underexpressed in a cell compared with a normal or standard cell.

As used herein “at least one target gene” refers to a nucleic acid sequence that encodes any portion of or all of a gene product and/or is operably linked to a nucleic acid encoding a gene product but does not necessarily comprise encoding sequence. By way of example, a nucleic acid sequence necessary for the expression of at least one gene product includes, but is not limited to, enhancers, promoters, regulatory sequences, start codons, stop codons, polyadenylation sequences, and/or encoding sequences. Expression levels of a polypeptide in a particular cell can be effected by, but not limited to, mutations, deletions and/or substitutions of various regulatory elements and/or non-encoding sequence in the cell genome. A gene may have one or more allelic variants, splice variants, derivative variants, substitution variants, deletion variants, truncation variants, and/or insertion variants, fusion polypeptides, orthologs, and/or interspecies homologs.

As used herein “gene product” refers to any portion or all of a protein or polypeptide encoded by at least one target gene. A gene product may be wild type or mutated. Gene products also include any polypeptide having or encoded by a target gene having one or more allelic variants, splice variants, derivative variants, substitution variants, deletion variants, truncation variants, and/or insertion variants, fusion polypeptides, orthologs, and/or interspecies homologs. By way of example, a gene product would include a protein in which part of all of the sequence of a polypeptide or gene encoding the protein is absent or not expressed in the cell. A gene product may be produced by a cell in a truncated form and the sequence of the truncated form may be wild type over the sequence of the truncate. A deletion may mean the absence of all or part of a gene or protein encoded by a gene. Additionally, some of a protein expressed in or encoded by a cell may be mutated while other copies of the same protein produced in the same cell may be wild type. By way of another example a mutation in a protein would include a protein having one or more amino acid differences in its amino acid sequence compared with wild type of the same protein.

As used herein “loci” refers to a specific location of a gene and/or a DNA sequence on a chromosome.

In one embodiment of the present invention, methods are provided for treating Crohn's disease in a human in need thereof, comprising administering denosumab to said human. In one aspect, the present invention embodies the use of denosumab for the treatment of Crohn's disease. In one aspect, the present invention embodies the use of denosumab in the manufacture of a medicament for the treatment of Crohn's disease.

Crohn's disease is a form of inflammatory bowel disease (IBD). It usually affects the intestines, but may occur anywhere from the mouth to the end of the rectum (anus).

Denosumab which is sold under the tradename Prolia® is a human monoclonal antibody for the treatment of osteoporosis, treatment-induced bone loss, bone metastases, rheumatoid arthritis, multiple myeloma, and giant cell tumor of bone. It was developed by the biotechnology company Amgen. Denosumab is designed to target RANKL (RANK ligand), a protein that acts as the primary signal for bone removal. In many bone loss conditions, RANKL overwhelms the body's natural defenses against bone destruction. Antibodies to RANKL are described, for instance, in U.S. Pat. No. 6,740,522 and U.S. Pat. No. 7,411,050. Denosumab is administered as a 60 mg (1 mL) injection every six months for osteoporosis treatment.

In one embodiment of the present invention, methods are provided for treating Crohn's disease in a human in need thereof, comprising administering to said human at least one compound selected from the group consisting of: an inhibitor and/or antagonist of tumor necrosis factor ligand, a T-cell co-stimulatory ligand, an IL-18 receptor antagonist and/or inhibitor, inducer of IL27 expression, anti-IL2 receptor mAb, chemokine (C—C motif) ligand 2 inhibitor and/or antagonist, estrogen related receptor alpha binding agent, galactosylceramidase, anti-Intercellular adhesion molecule 3 (ICAM3) mAb, anti-ICOS mAb, IL-23 receptor inhibitor and/or antagonist, Janus kinase 2 inhibitor, leucine-rich repeat kinase 2 inhibitor, mucin 1, cell surface associated inhibitor, signal transducer and activator of transcription 3 (acute-phase response factor) inhibitor, and tyrosine kinase 2 inhibitor.

In one embodiment, the human also has Celiac disease, irritable bowel syndrome and or inflammatory bowel disease.

In one embodiment, the compound is a tumor necrosis factor ligand antagonist.

In one embodiment, the tumor necrosis factor ligand is member 11 or receptor activator of nuclear factor kappa-B ligand (RANKL). In one embodiment, the tumor necrosis factor ligand is member 15.

In one embodiment, the antagonist is a monoclonal antibody. In one embodiment, the monoclonal antibody is humanized. In one embodiment, the monoclonal antibody is a human antibody. In one embodiment, the monoclonal antibody is denosumab or a functional fragment thereof.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′).sub.2, Fv), single chain (ScFv), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

A “monoclonal antibody” refers to a homogeneous antibody population wherein the monoclonal antibody is comprised of amino acids (naturally occurring and non-naturally occurring) that are involved in the selective binding of an antigen. Monoclonal antibodies are highly specific, being directed against a single antigenic site. The term “monoclonal antibody” encompasses not only intact monoclonal antibodies and full-length monoclonal antibodies, but also fragments thereof (such as Fab, Fab′, F(ab′).sub.2, Fv), single chain (ScFv), mutants thereof, fusion proteins comprising an antibody portion, humanized monoclonal antibodies, chimeric monoclonal antibodies, and any other modified configuration of the immunoglobulin molecule that comprises an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.). The term includes whole immunoglobulins as well as the fragments etc. described above under the definition of “antibody”.

“Altered antibody” refers to a protein encoded by an altered immunoglobulin coding region, which may be obtained by expression in a selected host cell. Such altered antibodies include engineered antibodies (e.g., chimeric, reshaped, humanized or vectored antibodies) or antibody fragments lacking all or part of an immunoglobulin constant region, e.g., Fv, Fab, or F(ab)2 and the like.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one (or more) human immunoglobulin(s). In addition, framework support residues may be altered to preserve binding affinity (see, e.g., Queen et al., Proc. Natl Acad Sci USA, 86:10029-10032 (1989), Hodgson et al., Bio/Technology, 9:421 (1991)). A suitable human acceptor antibody may be one selected from a conventional database, e.g., the KABAT® database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanised antibodies—see for example EP-A-0239400 and EP-A-054951.

The term “donor antibody” refers to an antibody (monoclonal, and/or recombinant) which contributes the amino acid sequences of its variable regions, CDRs, or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to an antibody (monoclonal and/or recombinant) heterologous to the donor antibody, which contributes all (or any portion, but preferably all) of the amino acid sequences encoding its heavy and/or light chain framework regions and/or its heavy and/or light chain constant regions to the first immunoglobulin partner. Preferably a human antibody is the acceptor antibody.

“CDRs” are defined as the complementarity determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs (or CDR regions) in the variable portion of an immunoglobulin. Thus, “CDRs” as used herein refers to all three heavy chain CDRs, or all three light chain CDRs (or both all heavy and all light chain CDRs, if appropriate). The structure and protein folding of the antibody may mean that other residues are considered part of the antigen binding region and would be understood to be so by a skilled person. See for example Chothia et al., (1989) Conformations of immunoglobulin hypervariable regions; Nature 342, p877-883.

CDRs provide the majority of contact residues for the binding of the antibody to the antigen or epitope. CDRs of interest in this invention are derived from donor antibody variable heavy and light chain sequences, and include analogs of the naturally occurring CDRs, which analogs also share or retain the same antigen binding specificity and/or neutralizing ability as the donor antibody from which they were derived.

A “functional fragment” is a partial heavy or light chain variable sequence (e.g., minor deletions at the amino or carboxy terminus of the immunoglobulin variable region) which retains the same antigen binding specificity and the same or similar neutralizing ability as the antibody from which the fragment was derived.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

In one embodiment of the present invention methods are provided for treating Crohn's disease in a human in need thereof, comprising administering a T-cell co-stimulator to said human. The T-cell co-stimulator may be an antibody that binds B7 related proteins. In one aspect the antibody is a human antibody that binds to B7 related protein-1 (B7RP-1).

In one embodiment of the present invention, methods are provided for treating Crohn's disease in a human in need thereof, comprising administering an inhibitor of signal transducer and activator of transcription 3 (STAT3) to said human.

The STAT3 inhibitors can be selected from one or more of the following: OPB-31121; OPB-51602; Bardoxolone methyl; Brivudine, and RESprote.

TNF inhibitors include, but are not limited to, monoclonal antibodies such as infliximab (Remicade), adalimumab (Humira), certolizumab pegol (Cimzia), and golimumab (Simponi), or with a circulating receptor fusion protein such as etanercept (Enbrel). Other TNF inhibitors include xanthine derivatives (e.g. pentoxifylline) and Bupropion.

IL-18 receptor proteins as well as proteins that bind to them, including antibodies, are described in U.S. Pat. Nos. 7,704,945, 7,141,393; 7,169,581; and 8,105,805. Polypeptides, including antibodies, that bind to IL-18 are described in U.S. Pat. Nos. 6,559,298; 6,589,764; and 7,767,207.

Examples of an anti-IL2 receptor mAb, include, but are not limited to, inolimomab, basiliximab, and daclizumab. Inolimomab is a mouse monoclonal antibody developed as an immunosuppressive drug against graft-versus-host disease. Its target is the alpha chain of the interleukin-2 receptor. Basiliximab (trade name Simulect) is a chimeric mouse-human monoclonal antibody to the a chain (CD25) of the IL-2 receptor of T cells. It is used to prevent rejection in organ transplantation, especially in kidney transplants. Daclizumab (trade name Zenapax) is a therapeutic humanized monoclonal antibody to the alpha subunit of the IL-2 receptor of T cells. It is used to prevent rejection in organ transplantation, especially in kidney transplants.

Chemokine (C—C motif) ligand 2 (CCL2) also known as monocyte chemotactic protein-1 (MCP-1) or small inducible cytokine A2 is a protein that in humans is encoded by the CCL2 gene. CCL2 is a small cytokine belonging to the CC chemokine family. CCL2 recruits monocytes, memory T cells, and dendritic cells to sites of tissue injury, infection, and inflammation. CCL2 plays a significant role in the CCL2/CCR2 pathway in lipoatrophy-induced diabetes. (Yang, et al. Diabetologia. 2009 May; 52(5):972-81).

The orphan nuclear receptor estrogen-related receptor alpha (ERRalpha) has been implicated in the development of various human malignancies, including breast, prostate, ovary, and colon cancer. SR16388 (21-[2-(N,N-Dimethylamino)ethyl]oxy-7a-methyl-19-norpregna-1,3,5(10),17(20)-tetraen-3-ol citrate salt) is an orally active compound that belongs to the antiestrogen class of therapeutic agents. SR16388 is a potent and selective inhibitor of human ERRα, which does not bind estrogen (E2). SR16388 is represented by the following formula (Duellman, et al Biochem Pharmacol. 2010 Sep. 15; 80(6): 819-826).

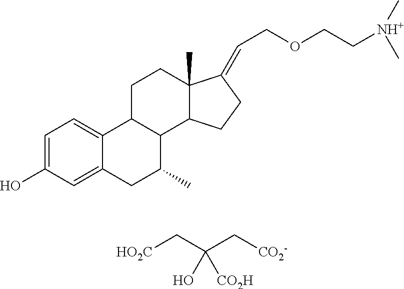

Galactosylceramidase (or galactocerebrosidase) is an enzyme that in humans is encoded by the GALC gene. Galactosylceramidase is an enzyme which removes galactose from ceramide derivatives (galactocerebrosides). Galactosylceramidase is a lysosomal protein which hydrolyzes the galactose ester bonds of galactocerebroside, galactosylsphingosine, lactosylceramide, and monogalactosyldiglyceride.

Janus kinase inhibitor is a class of medicines that function by inhibiting the effect of one or more of the Janus kinase family of enzymes (JAK1, JAK2, JAK3, TYK2), interfering with the JAK-STAT signaling pathway. Some JAK2 inhibitors are under development for the treatment of polycythemia vera, essential thrombocythemia, and myeloid metaplasia with myelofibrosis. Some inhibitors of JAK2 are in clinical trials, e.g. for psoriasis. JAK3 is also being targeted for a variety of inflammatory diseases, and one has had good results in a phase II trial for rheumatoid arthritis. Janus kinase inhibitors include but are not limited to:

Lestaurtinib against JAK2, for acute myelogenous leukemia (AML) which is represented by the formula:

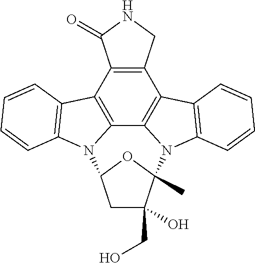 [SMILES: C[C@]12O[C@H](C[C@]1(O)CO)n1c3ccccc3c3c4c(c5c6ccccc6n2c5c31)CNC4=O]

Tofacitinib (previously called tasocitinib) (CP-690550) against JAK3 for psoriasis, and rheumatoid arthritisis shown in the following formula:

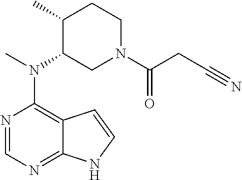 [SMILES: C[C@@H]1CCN(C(=O)CC#N)C[C@@H]1N(C)c1ncnc2[nH]ccc12]

Ruxolitinib against JAK1/JAK2 for psoriasis, myelofibrosis, and rheumatoid arthritis is shown in the following formula:

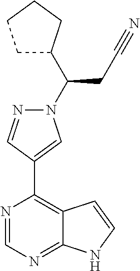

Pacritinib (SB1518) against JAK2 for relapsed lymphoma, advanced myeloid malignancies, myelofibrosis and CIMF Phase II results for polycythemia vera and thrombocythemia myelofibrosis is represented by the following formula:

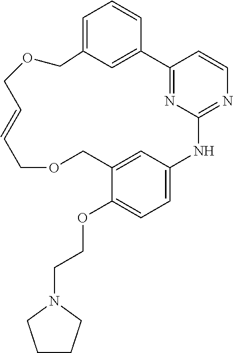 [SMILES: C1=C/COCc2cc(ccc2OCCN2CCCC2)Nc2nccc(n2)-c2cccc(c2)COC/1]

CYT387 against JAK2 for myeloproliferative disorders is shown in the following formula:

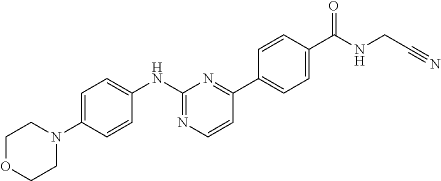 [SMILES: N#CCNC(=O)c1ccc(-c2ccnc(Nc3ccc(N4CCOCC4)cc3)n2)cc1]

Baricitinib (LY3009104, INCB28050) against JAK1/JAK2 for rheumatoid arthritis is shown in the following formula.

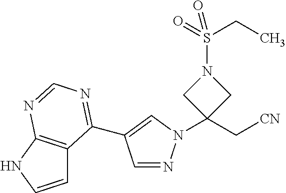 [SMILES: CCS(=O)(=O)N1CC(CC#N)(n2cc(-c3ncnc4[nH]ccc34)cn2)C1]

TG101348 (SAR302503) is an orally available inhibitor of Janus kinase 2 (JAK-2) developed for the treatment of patients with myeloproliferative diseases including myelofibrosis. TG101348 acts as a competitive inhibitor of protein kinase JAK-2 with IC50=6 nM; related kinases FLT3 and RET are also sensitive, with IC₅₀=25 nM and IC₅₀=17 nM, respectively. Significantly less activity was observed against other tyrosine kinases including JAK3 (IC₅₀=169 nM). In treated cells the inhibitor blocks downstream cellular signaling (JAK-STAT) leading to suppression of proliferation and induction of apoptosis. TG101348 is represented by the following formula:

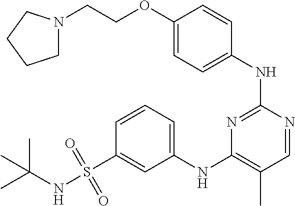 [SMILES: Cc1cnc(Nc2ccc(OCCN3CCCC3)cc2)nc1Nc1cccc(S(=O)(=O)NC(C)(C)C)c1]

In one embodiment of the present invention methods are provided for treating multiple sclerosis (MS) in a human in need thereof, comprising administering a STAT3 inhibitor to said human.

In one embodiment of the present invention, methods are provided for repositioning a pharmaceutical, comprising the steps of:
selecting at least one target gene, gene product, or loci associated with the treatment of at least one first disease, trait and/or phenotype by said pharmaceutical,
identifying at least one second disease, trait and/or phenotype associated with least one target gene, gene product, or loci of (a) using genome-wide associated studies,
selecting at least one second disease, trait and/or phenotype based on step (b) for treatment with said pharmaceutical.

In one aspect, the methods further comprise determining expression or overexpression and/or amplification of said first target gene, gene product, or loci in said second disease, trait and/or phenotype. In another aspect, the methods of the present invention further comprise identifying additional data and/or experimental support for target gene in said second disease, trait and/or phenotype. In another embodiment, the methods comprise identifying at least one SNPs in said target gene in said second disease, trait and/or phenotype.

In another embodiment of the present invention, methods are provided for validating or invalidating a first therapeutic indication of a pharmaceutical comprising matching all GWAS associated diseases, traits and/or phenotypes of at least one target gene associated with said first therapeutic indication and determining if at least one GWAS associated disease, trait and/or phenotype of said target gene is associated with said first therapeutic indication.

In another embodiment of the present invention, methods are provided for selecting a therapeutic agent for treatment or prevention of a disease comprising the steps of:
selecting at least one target gene or gene product associated with at least one disease, trait and/or phenotype by at least one genome-wide associated study; optionally determining if said target gene or gene product is small molecule druggable, secreted protein, antibody accessible, or modifiable by some other modality including anti-sense oligos, gene therapy, aptamers;
determining if said target is under expressed or over expressed for a particular disease, trait and/or phenotype;
selecting a therapeutic agent that is an agonist to said target gene or gene product if said gene is under reduced functionality in said disease and/or trait of step (a) and selecting a therapeutic agent that is an antagonist to said target gene or gene product if said gene is under increased functionality in a particular disease and/or trait.

In another embodiment of the present invention, methods are provided for aiding a human to reduce the frequency of smoking or stop smoking cigarettes comprising administering a dopamine beta-hydroxylase inhibitor to said human. Inhibitors of dopamine beta-hydroxylase are described in U.S. Pat. Nos. 4,487,761 and 5,538,988 as well as US Patent Publication No. 20100105748. In some aspects, the dopamine beta-hydroxylase inhibitor is Nepicastat®. In some aspects, the dopamine beta-hydroxylase inhibitor is disulfuram. In some aspects, the dopamine beta-hydroxylase inhibitor is selected from S-5-(aminomethyl)-1-[(2S)-5,7-difluoro-1,2,3,4-tetrahydronaphthalen-2-yl]-1,3-dihydro-2H-imidazole-2-thione and 1,1′,1″,1′″-[disulfanediylbis(carbonothioylnitrilo)]tetraethane or pharmaceutically acceptable salts thereof.

Nepicastat® (5-(aminomethyl)-1-[(2S)-5,7-difluoro-1,2,3,4-tetrahydronaphthalen-2-yl]-1,3-dihydro-2H-imidazole-2-thione) is an inhibitor of dopamine beta-hydroxylase, an enzyme that catalyzes the conversion of dopamine to norepinephrine. The chemical structure of Nepicastat® is shown below as Formula 1

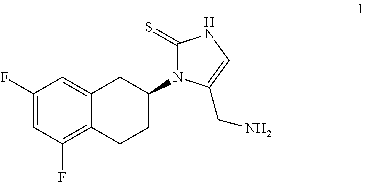 [SMILES: NCc1c[nH]c(=S)n1[C@H]1CCc2c(F)cc(F)cc2C1]

Disulfiram (1,1′,1″,1′″-[disulfanediylbis(carbonothioylnitrilo)]tetraethane) is used to support the treatment of chronic alcoholism by producing an acute sensitivity to alcohol. Trade names for disulfiram in different countries are Antabuse® and Antabus®. The chemical structure of disulfiram is shown below as Formula 2

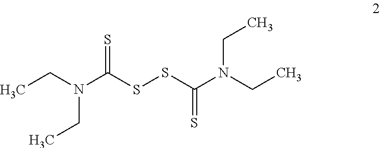 [SMILES: CCN(CC)C(=S)SSC(=S)N(CC)CC]

In another embodiment of the present invention, methods are provided for treating Type 1 diabetes in a human comprising administering IL2 or an IL2 mimetic to said human. In some instances, the IL2 or IL2 mimetic is selected from aldesleukin and Proleukin®.

Proleukin® is manufactured by Chiron Corporation of Emeryville, Calif. The IL-2 in this formulation is a recombinantly produced human IL-2 mutein, called aldesleukin, which differs from the native human IL-2 sequence in having the initial alanine residue eliminated and the cysteine residue at position 125 replaced by a serine residue (referred to as des-alanyl-1, serine-125 human interleukin-2). This IL-2 mutein is expressed from E. coli, and subsequently purified by diafiltration and cation exchange chromatography as described in U.S. Pat. No. 4,931,543. The IL-2 formulation marketed as Proleukin is supplied as a sterile, white to off-white preservative-free lyophilized powder in vials containing 1.3 mg of protein (22 MIU).

Aldesleukin is a man-made protein that has the same actions as native human interleukin-2 (IL-2). Interleukins are the messengers by which white blood cells communicate with each other to coordinate inflammation and immunity. Among its actions, IL-2 increases the number and activities of certain types of white blood cells called lymphocytes, monocytes, and macrophages that are involved in inflammation and immunity. For example, lymphocytes fight viral infections, regulate the immune system, and fight cancers. Aldesleukin in given only by injection. Aldesleukin was FDA approved in 1992.

In another embodiment of the present invention, methods are provided for treating Crohn's disease in a human, comprising administering an inhibitor and/or antagonist of tumor necrosis factor ligand, member 15, to said human. In one aspect the inhibitor and/or antagonist is a monoclonal antibody.

In another embodiment of the present invention, methods are provided for treating inflammatory bowel syndrome in a human comprising administering an inhibitor and/or antagonist of tumor necrosis factor ligand, member 15, to said human. In one aspect the inhibitor and/or antagonist is a monoclonal antibody.

In another embodiment of the present invention, methods are provided for treating Crohn's disease in a human, comprising administering an inhibitor and/or antagonist of tumor necrosis factor ligand, member 11, to said human. In one aspect the inhibitor and/or antagonist is a monoclonal antibody. In one aspect the monoclonal antibody is denosumab.

Receptor activator of nuclear factor kappa-B ligand (RANKL), also known as tumor necrosis factor ligand superfamily member 11 (TNFSF11), TNF-related activation-induced cytokine (TRANCE), osteoprotegerin ligand (OPGL), and osteoclast differentiation factor (ODF), is a protein that in humans is encoded by the TNFSF11 gene. Critical for adequate bone metabolism, this surface-bound molecule (also known as CD254) found on osteoblasts serves to activate osteoclasts, which are the cells involved in bone resorption.

RANKL is a member of the tumor necrosis factor (TNF) cytokine family which is a ligand for osteoprotegerin and functions as a key factor for osteoclast differentiation and activation. RANKL also has a function in the immune system, where it is expressed by T helper cells and is thought to be involved in dendritic cell maturation. This protein was shown to be a dendritic cell survival factor and is involved in the regulation of T cell-dependent immune response. T cell activation was reported to induce expression of this gene and lead to an increase of osteoclastogenesis and bone loss. This protein was shown to activate antiapoptotic kinase AKT/PKB through a signaling complex involving SRC kinase and tumor necrosis factor receptor-associated factor 6 (TRAF6), which indicated this protein may have a role in the regulation of cell apoptosis.

STAT3 inhibitors include, but are not limited to, OPB-31121 (A Novel STAT3 Inhibitor OPB-31121 Induces Tumor-Specific Growth Inhibition in a Wide Range of Hematopoietic Malignancies without Growth Suppression of Normal Hematopoietic Cells 53^{rd} ASH Annual Meeting and Exposition December, 2011 absrtact); OPB-51602 (Clinicaltrial.gov); Bardoxolone methyl; Brivudine, and RESprote. Various STAT3 inhibitors are described in US2011/0172429, US20110223661, US20110312,984, and US20120035114.

Bardoxolone methyl can be described by the following formula.

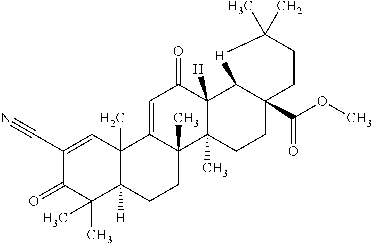

Brivudine can be described by the following formula.

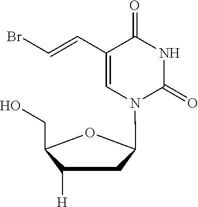

In another embodiment of the present invention, methods are provided for treating Type II diabetes in a human comprising administering an agonist to melatonin receptor 1B to said human. In one aspect, said agonist is melatonin.

In another embodiment of the present invention, methods are provided for treating psoriasis in a human comprising administering to said human antagonist of interleukin 13. In one aspect, the antagonist is a monoclonal antibody to IL-13.

In another embodiment of the present invention, methods are provided for treating Behcet's disease in a human comprising administering an inhibitor and/or antagonist of IL-10 to said human. Monoclonal antibodies to human IL-10 are described in WO2011064399 and WO2011 064398.

In another embodiment of the present invention, methods are provided for treating essential tumor in a human, comprising administering an inhibitor to leucine rich repeat and Ig domain containing 1 (LINGO) to said human. In one aspect the inhibitor of the inhibitor of leucine rich repeat and Ig domain containing 1 (LINGO) is Biib-033.

In another embodiment of the present invention, methods are provided for treating Alzheimer's disease in a human comprising administering a compound that upregulates clusterin to said patient. In one aspect the compound that upregulates clusterin is selected from Valproate and Vorinostat.

Anti-clusterin antibodies and antigen binding fragment are described are described in WO2011063523.

In another embodiment of the present invention, methods are provided for treating Alzheimer's disease in a human comprising administering a compound that modulates complement component (3b/4b) receptor 1. In one aspect the compound that modulates complement component (3b/4b) receptor 1 is selected from Candida hp, CDx-1135, Eti-204, and Eti-211.

In another embodiment of the present invention, methods are provided for treating Alzheimer's disease in a human comprising administering to said human a clusterin inhibitor. In one aspect the clusterin inhibitor is selected from Ab-16b5 and Clustirsen.

In another embodiment of the present invention, methods are provided for treating Crohn's disease and/or Celiac disease in a human comprising administering a T-cell co-stimulatory ligand to said human. In one aspect the T-cell co-stimulator is an antibody that binds B7 related proteins. T-cell co-stimulators are described in U.S. Pat. Nos. 7,030,219 and 7,560,540, 7,101,550, 7,358,354, 7,723,479, 7,414,122, 7,595,048, 7,563,896, 7,488,802, and 7,432,351 as well as WO 2010/027828, WO 2010/098788, WO 2010/027423. In one aspect, the T-cell co-stimulator is AMG-557, which is a human antibody that binds to B7 related protein (B7RP-1).

In another embodiment of the present invention, methods are provided for treating Crohn's disease and/or inflammatory bowel disease (IBD) in a human comprising administering an IL-18 receptor antagonist and/or inhibitor to said human. In one aspect, the IL-18R antagonist/inhibitor is a monoclonal antibody.

In another embodiment of the present invention, methods are provided for treating Crohn's disease and/or IBD in a human comprising administering an inducer of IL27 expression to said human. In one aspect, the inducer of IL27 expression is Rpi-78m. Methods of using Rpi-78m are described U.S. Pat. No. 8,034,777, titled “Modified anticholinergic neurotoxins as modulators of the autoimmune reaction,” describes a composition of matter and method of its use for the treatment of multiple sclerosis in humans. The composition is a modified anticholinergic alpha-neurotoxin.

In another embodiment of the present invention, methods are provided for treating primary biliary cirrhosis in a human comprising administering a compound that modulates IL12A to said human. In one aspect the compound that modulates IL12A is selected from briakinumab; ustekinumab, an IL-12 expressing plasmid, Egen-001; and Interleukin-12, including HemaMax.

IL-12 antibodies are described in U.S. Pat. No. 7,887,807. EGEN-001 (E1), an IL-12 expressing plasmid formulated with a novel gene delivery system, stimulates natural killer cells, IFN-′γ secretion, and T-helper 1 response, inhibits tumor neovascularization, and has potent antitumor activity in preclinical models of ovarian cancer.

In another embodiment of the present invention, methods are provided for treating Crohn's disease in a human comprising administering an anti-IL2 receptor mAb to said human.

In another embodiment of the present invention, methods are provided for treating Type II diabetes in a human comprising administering azimilide to said human. Azimilide is a class III antiarrhythmic drug (used to control abnormal heart rhythms). The agents from this heterogeneous group have an effect on the repolarization, they prolong the duration of the action potential and the refractory period. Also they slow down the spontaneous discharge frequency of automatic pacemakers by depressing the slope of diastolic depolarization. They shift the threshold towards zero or hyperpolarize the membrane potential. Although each agent has its own properties and will have thus a different function. Azilimide has the following chemical structure and IUPAC name:

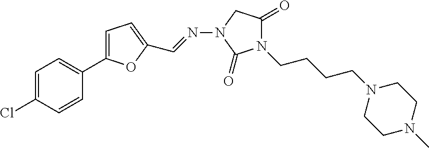 [SMILES: CN1CCN(CCCCN2C(=O)CN(/N=C/c3ccc(-c4ccc(Cl)cc4)o3)C2=O)CC1]

1-({(E)[5-(4-chlorophenyl)furan-2-yl]methylidene}amino)-3-[4-(4-methylpiperazin-1-yl)butyl]imidazolidine-2,4-dione.

In another embodiment of the present invention, methods are provided for treating Type I diabetes in a human comprising administering ACN-189 and/or AEN-071 to said human.

In another embodiment of the present invention, methods are provided for treating systemic lupus erythromatosus in a human comprising administering an inhibitor of TNFSF4 to said human. In one aspect the inhibitor of TNFSF4 is Oxelumab. Oxelumab is an IgG1 monoclonal antibody with human monoclonal γ-chain and human monoclonal κ-chain. Oxelmab binds to human antigen OX-40 ligand which is a member of Tumor Necrosis Factor Ligand superfamily, member 4.

In another embodiment of the present invention, methods are provided for treating coronary heart disease in a human comprising administering an CXCL12-specific inhibitor to said human. In one aspect the CXCL12-specific inhibitor is NOX-A12. NOX-A12 is an L:-enantiomeric RNA oligonucleotide.

In another embodiment of the present invention, methods are provided for treating idiopathic pulmonary fibrosis, comprising administering at least one telomerase reverse transcriptase inhibitor.

Examples of CCR4 inhibitors such as N-[(3-{[3-{[(5-Chloro-2-thienyl)sulfonyl]amino}-4-(methyloxy)-1H-indazol-1-yl]methyl}phenyl)methyl]-2-hydroxy-2-methylpropanamide are described in WIPO international publication WO2010/097395 and US Patent Publication No. 20100216860 A1.

Examples of PDE4 inhibitors such as GSK-256066 (6-({3-[(dimethylamino)carbonyl]phenyl}sulfonyl)-8-methyl-4-{[3-methyloxy]phenylamino}-3-quinolinecarboxamide) can be found in PCT/EP04/05494—WIPO publication WO2004103998 A1 and U.S. Pat. Nos. 7,572,915 and 7,566,786 and GSK-356278 (5-(5-((2,4-dimethylthiazol-5-yl)methyl)-1,3,4-oxadiazol-2-yl)-1-ethyl-N-(tetrahydro-2H-pyran-4-yl)-1H-pyrazolo[3,4-b]pyridin-4-amine) can be found in PCT/EP03/14867—WIPO publication WO 2004056823 A1 and U.S. Pat. No. 7,528,148.

PTK2 protein tyrosine kinase 2 inhibitors (or FAK inhibitors) such as (2-((5-chloro-2-((1-isopropyl-3-methyl-1H-pyrazol-5-yl)amino)pyridin-4-yl)amino)-N-methoxybenzamide) are described and claimed in PCT/US2009/062163—WIPO Publication WO/2010/062578 and US Publication Nos. US 20100113475 A1 and US 20110269774 A1.

Examples of opoid receptor agonists such as GSK1521498 (N-((3,5-difluoro-3′-(4H-1,2,4-triazol-3-yl)[1,1′-biphenyl]-4-yl)methyl)-2,3-dihydro-1H-inden-2-amine) are described in PCT/US2007/075422—WIPO publication WO 2008021849 A2 and US Publication No. US 20100113512 A1.

Examples of antibodies that bind and neutralize NOGO such as GSK-1223249 are described in U.S. Pat. Nos. 7,780,964 and 7,988,964. Antibodies and single chain antibodies that bind NOGO receptor are described in U.S. Pat. No. 7,973,139.

“NOGO” refers to any NOGO polypeptide, including variant forms. This includes, but is not limited to, NOGO-A having 1192 amino acid residues (GenBank accession no. AJ251383); NOGO-B, a splice variant which lacks residues 186 to 1004 in the putative extracellular domain (GenBank accession no. AJ251384) and a shorter splice variant, NOGO-C, which also lacks residues 186 to 1004 and also has smaller, alternative amino terminal domain (GenBank accession no. AJ251385) (Prinjha R et al (2000) Nature 403, 383-384; Chen M S et al (2000) Nature 403). All references to “NOGO” herein is understood to include any and all variant forms of NOGO such as NOGO-A and the splice variants described, unless a specific form is indicated.

In another embodiment of the present invention, new therapeutic indications are provided for drugs and biotherapeutics according to Table 1. The column designated “New suggested indication” of the Table 1 provides new therapeutic indication determined by the methods of the present invention for the corresponding drugs and classes of therapy listed in the column designated “All drugs” of Table 1.

TABLE 1

| | | | No. | | | | |
|---|---|---|---|---|---|---|---|
| | | | of | Strongest | | | |
| | | | asso- | SNP-Risk | | | |
| | | GWAS | ciated | Allele | New suggested | | |
| Gene | Description | phenotype | genes | (frequency) | indication | Current indications | All drugs |
| ABCA1 | ATP-binding | Coronary | 78 | rs2515629-A | Coronary heart | Alzheimer's disease; Atherosclerosis; | Alzheimer's disease therapy, Madera BioSciences; |
| | cassette, sub- | heart disease | | (0.78) | disease | Cholelithiasis; Diabetes, Type 2; | Aramchol; RG-72 |
| | family A (ABC1), | | | | | Hyperlipidaemia, general; Steatohepatitis | |
| | member 1 | | | | | | |
| ABCA4 | ATP-binding | Dialysis- | 31 | rs17110736-? | Dialysis-related | Macular degeneration, early onset | StarG |
| | cassette, sub- | related | | (0.35) | mortality | | |
| | family A (ABC1), | mortality | | | | | |
| | member 4 | | | | | | |
| ACHE | acetylcholinesterase | Type 2 | 61 | rs7636-A | Type 2 diabetes | Alzheimer's disease; Arthritis, osteo; Arthritis, | 60714; GLN-1062; KW-5092; NP-0361; P26; TAK-802; |
| | | diabetes | | (0.06) | | rheumatoid; Attention deficit hyperactivity | Yissum Project No. B-1045; Acetylcholinesterase, |
| | | | | | | disorder; Cognitive disorder, unspecified; | Protalix; Acotiamide hydrochloride; Donepezil |
| | | | | | | Dementia, Lewy body; Dementia, Parkinson's | hydrochloride; Donepezil hydrochloride RDT; |
| | | | | | | disease; Dementia, presenile; Dementia, senile, | Donepezil hydrochloride jelly; Donepezil |
| | | | | | | general; Dementia, vascular; Depression, | hydrochloride, APR; Donepezil hydrochloride, SR; |
| | | | | | | general; Down's syndrome; Dyspepsia; | Donepezil transdermal patch; Eptastigmine; |
| | | | | | | Fibromyalgia; Gastritis; Huntington's disease; | Galantamine hydrobromide; Galantamine, CR capsule; |
| | | | | | | Ischaemia, cerebral; Migraine; Motility | Huperzine A; Itopride hydrochloride; Ladostigil |
| | | | | | | dysfunction, GI, general; Poisoning, | tartrate; Memantine + donepezil, Adamas; |
| | | | | | | organophosphate; Radio/chemotherapy- | Methanesulfonyl fluoride, Senexta; Metrifonate; |
| | | | | | | induced injury, general; Schizophrenia; Urinary | Mimopezil; Mimopezil LA; Mimopezil, implant; |
| | | | | | | retention | Physostigmine, Forest; Physostigmine, Klinge; |
| | | | | | | | Rivastigmine tartrate; Rivastigmine tartrate, TDS; |
| | | | | | | | Tacrine; Tacrine, OROS; Thiatolseri |
| ACVR2A | activin A | Response to | 75 | rs7584099-G | Response to | Anaemia, radio/chemotherapy-induced; | EDF, Ajinomoto; Sotaterce |
| | receptor, type IIA | statin therapy | | (0.41) | statin therapy | Anaemia, renal disease-induced; Cancer, bone; | |
| | | | | | | Cancer, general; Cancer, myeloma; | |
| | | | | | | Osteoporosis | |
| ADRA2A | adrenergic, | Fasting | 18 | rs10885122-G | Fasting glucose- | Addiction, narcotic/opiate; Anxiety, general; | ADX-415; Brimonidine-DDS; Org-4419; Parkinson's |
| | alpha-2A-, | glucose- | | (0.87) | related traits | Apnoea; Attention deficit hyperactivity | therapy, Heptares; Bimatoprost/brimonidine |
| | receptor | related traits | | | | disorder; Conjunctivitis, inflammatory; | tartrate/timolol; Brimonidine; Brimonidine + |
| | | | | | | Depression, general; Depression, major | timolol, Allergan; Brimonidine tartrate, Eye Therapies; |
| | | | | | | depressive disorder; Dyskinesia, general; | Brinzolamide + brimonidine, Alcon; Clonidine ER, |
| | | | | | | Dyskinesia, levodopa-induced; Fibromyalgia; | once-daily, Tris Pharma; Clonidine SR, Addrenex; |
| | | | | | | Glaucoma; Heart failure; Hypertension, | Clonidine, Alza; Clonidine, BioAlliance Pharma; |
| | | | | | | general; Macular degeneration, age-related, | Clonidine, Biovail; Clonidine, Fujisawa; Clonidine, |
| | | | | | | general; Menopausal symptoms, unspecified; | Hisamitsu; Clonidine, Jazz; Clonidine, topical, Arcion; |
| | | | | | | Neuropathy, diabetic; Obsessive-compulsive | Dorzolamide HCl/brimonidine tartrate/timolol, |
| | | | | | | disorder; Pain, cancer; Pain, general; Pain, | Allergan; Fipamezole; Guanfacine; Guanfacine |
| | | | | | | neuropathic; Panic disorder; Parkinson's | carrierwave; Guanfacine, ER, Shire; Lofexidine; |
| | | | | | | disease; Post-traumatic stress disorder; | Mirtazapine; Mirtazapine, immediate-release; |
| | | | | | | Radio/chemotherapy-induced mucositis; | Prazosin; Prazosin, Al |
| | | | | | | Retinal detachment; Retinitis pigmentosa; | |
| | | | | | | Surgery adjunct | |
| AHR | aryl hydrocarbon | Coffee | 3 | rs6968865-T | Coffee | Cancer, solid, general | 5F-DF-2 |
| | receptor | consumption | | (0.593) | consumption | | |
| AKT3 | v-akt murine | RR interval | 4 | rs4132509-A | RR interval | Cancer, solid, general | LY-25030 |
| | thymoma viral | | | (0.21) | | | |
| | oncogene | | | | | | |
| | homolog 3 | | | | | | |
| | (protein kinase B, | | | | | | |
| | gamma) | | | | | | |
| AKT3 | v-akt murine | Diabetic | 34 | rs10927101-A | Diabetic | Cancer, solid, general | LY-25030 |
| | thymoma viral | retinopathy | | (0.38), | retinopathy | | |
| | oncogene | | | rs476141-A | | | |
| | homolog 3 | | | (0.51) | | | |
| | (protein kinase B, | | | | | | |
| | gamma) | | | | | | |
| ALDH2 | aldehyde | Esophageal | 15 | rs671-A (NR) | Esophageal | Alcohol intolerance | Fomepizole, Rapt |
| | dehydrogenase 2 | cancer | | | cancer | | |
| | family | | | | | | |
| | (mitochondrial) | | | | | | |
| ALDH2 | aldehyde | Upper | 7 | rs4767364-A | Upper | Alcohol intolerance | Fomepizole, Rapt |
| | dehydrogenase 2 | aerodigestive | | (0.29) | aerodigestive | | |
| | family | tract cancers | | | tract cancers | | |
| | (mitochondrial) | | | | | | |
| APOA1 | apolipoprotein A-I | Coronary | 78 | rs964184-G | Coronary heart | Acute coronary syndrome; Alzheimer's disease; | 48266; CER-001; CER-522; CRD-5; CRD-510; ET-216; |
| | | heart disease | | (0.13) | disease | Angina, general; Atherosclerosis; | ET-642; QRS-10-001; QRS-5-005; RVX-208; RVX-408; |
| | | | | | | Hypercholesterolaemia; Hyperlipidaemia, | Rev-D4F; Apolipoprotein A-I upregulators, |
| | | | | | | general; Ischaemia, general; Restenosis; Sepsis; | GlaxoSmithKline; Apolipoprotein A1 + interferon alfa- |
| | | | | | | Stenosis, aortic valve | 5, Digna Biotech; Apolipoprotein AI, SemBioS |
| APOB | apolipoprotein B | Erectile | 23 | rs219553-? | Erectile | Atherosclerosis; Hypercholesterolaemia | 49694; BI-204; PRO-040201; SPC-4955; MiApoB10, |
| | (including Ag(x) | dysfunction | | (0.42) | dysfunction and | | Amsterdam Molecular Therapeutics; Mipomersen |
| | antigen) | and prostate | | | prostate cancer | | sodium; ShApoB |
| | | cancer | | | treatment | | |
| | | treatment | | | | | |
| APOB | apolipoprotein B | Response to | 75 | rs541041-G | Response to | Atherosclerosis; Hypercholesterolaemia | 49694; BI-204; PRO-040201; SPC-4955; MiApoB10, |
| | (including Ag(x) | statin therapy | | (0.16) | statin therapy | | Amsterdam Molecular Therapeutics; Mipomersen |
| | antigen) | | | | | | sodium; ShApoB |
| APOB | apolipoprotein B | Hyper- | 5 | rs4635554-G | Hypertriglyceridemia | Atherosclerosis; Hypercholesterolaemia | 49694; BI-204; PRO-040201; SPC-4955; MiApoB10, |
| | (including Ag(x) | triglyceridemia | | (0.31) | | | Amsterdam Molecular Therapeutics; Mipomersen |
| | antigen) | | | | | | sodium; ShApoB |
| APOC3 | apolipoprotein C- | Coronary | 78 | rs964184-G | Coronary heart | Hyperlipidaemia, general | ISIS-APOCIII |
| | III | heart disease | | (0.13) | disease | | |
| APOE | apolipoprotein E | Longevity | 23 | rs2075650-A | Longevity | Acute coronary syndrome; Alzheimer's disease; | AEM-18; AEM-28; Alzheimer's disease therapy, |
| | | | | (0.74) | | Colitis, general; Hypercholesterolaemia; | Madera BioSciences; COG-112; Apolipoprotein- |
| | | | | | | Multiple sclerosis, general; Multiple sclerosis, | E2, GlaxoSmith |
| | | | | | | progressive, general; Multiple sclerosis, | |
| | | | | | | relapsing-remitting; Spinal cord injury | |
| APOE | apolipoprotein E | Alzheimer's | 28 | rs157580-? | Alzheimer's | Acute coronary syndrome; Alzheimer's disease; | AEM-18; AEM-28; Alzheimer's disease therapy, |
| | | disease | | (NR), | disease | Colitis, general; Hypercholesterolaemia; | Madera BioSciences; COG-112; Apolipoprotein- |
| | | | | rs2075650-? | | Multiple sclerosis, general; Multiple sclerosis, | E2, GlaxoSmith |
| | | | | (0.15), | | progressive, general; Multiple sclerosis, | |
| | | | | rs2075650-? | | relapsing-remitting; Spinal cord injury | |
| | | | | (0.90), | | | |
| | | | | rs2075650-G | | | |
| | | | | (0.14), | | | |
| | | | | rs2075650-G | | | |
| | | | | (0.20), | | | |
| | | | | rs4420638-? | | | |
| | | | | (NR), rs6859- | | | |
| | | | | A (NR) | | | |
| APOE | apolipoprotein E | Alzheimer's | 4 | rs429358-? | Alzheimer's | Acute coronary syndrome; Alzheimer's disease; | AEM-18; AEM-28; Alzheimer's disease therapy, |
| | | disease | | (NR) | disease | Colitis, general; Hypercholesterolaemia; | Madera BioSciences; COG-112; Apolipoprotein- |
| | | biomarkers | | | biomarkers | Multiple sclerosis, general; Multiple sclerosis, | E2, GlaxoSmith |
| | | | | | | progressive, general; Multiple sclerdsis, | |
| | | | | | | relapsing-remitting; Spinal cord injury | |
| AR | androgen | LDL | 42 | rs5031002-A | LDL cholesterol | Acne; Alopecia, androgenic; Anaemia, | 11096; 52649; 54962; 60003; ADR-L09; APC-100; ASP- |
| | receptor | cholesterol | | (0.02) | | unspecified; Benign prostatic hyperplasia; | 9521; BLACE; CH-4933468; CH-5137291; CNF-316 |
| | | | | | | Cachexia; Cancer, breast; Cancer, prostate; | series; DHEA + acolbifene, Endoceutics; EZN-4176; |
| | | | | | | Cognitive disorder, unspecified; Contraceptive, | GLPG-0492; GTx-027; HE-3235; LGD-4033; LPCN-1098; |
| | | | | | | female; Contraceptive, male; Diabetes, Type 2; | LY-2452473 + tadalafil, Lilly; MDV-3100; ODM-201; PA- |
| | | | | | | Dystrophy, Duchenne's muscular; | 109; PS-178990; Permixon, once-daily, Pierre; |
| | | | | | | Endometriosis; Fibrosis, uterine; Heart failure; | Pharmaprojects No. 1381; Pharmaprojects No. 3606; |
| | | | | | | Hepatitis, non-infectious; Hirsutism; Hormone | RAD-140; SARD programme, Aragon; SARMs, GTx-2; |
| | | | | | | replacement therapy; Hypogonadism; | VAL-201; ZD-3980; Abarelix; Androgen antagonists, |
| | | | | | | Impotence; Labour, induction; Menopausal | BMS-2; Androgen inhibitor, AndroBio; Androgen |
| | | | | | | symptoms, unspecified; Osteoporosis; | receptor antagonists, BMS-3; Bicalutamide; |
| | | | | | | Sarcopenia; Seborrhoea; Sex-chromosome | Chlormadinone acetate + ethinyl estradiol; |
| | | | | | | abnormality, Turner's syndrome; Sexual | Contraceptive, BioSante; Cortodoxone; Cyproterone |
| | | | | | | dysfunction, female; Sexual dysfunction, male; | acetate + estradiol valerate, Bayer; Cyproterone |
| | | | | | | Xerophthalmia | acetate + ethinylestradiol, Douglas; |
| | | | | | | | Dehydroepiandrosterone, Bayer; |
| | | | | | | | Dehydroepiandrosterone, Endoceutics; |
| | | | | | | | Dihydrotestosterone, Unimed; Enclomiphene citrate; |
| | | | | | | | Flutamide; Galeterone; Nilutamide; Ostarine; |
| | | | | | | | Oxandrolone; Oxendolone; Oxymetholone, Unimed; |
| | | | | | | | Prostate cancer siRNA, SeleXel; Selective androgen |
| | | | | | | | receptor modulators, GTx; Sodium prasterone |
| | | | | | | | sulfate, S-P; Testosterone enanthate; Testosterone gel |
| | | | | | | | (intranasal, low-dose), Trimel BioPharma; |
| | | | | | | | Testosterone gel, Antares-2; Testosterone gel, Trimel |
| | | | | | | | BioPharma; Testosterone ophthalmic solution, |
| | | | | | | | Allergan; Testosterone patch, female; Testosterone |
| | | | | | | | transdermal, ProStrakan; Testosterone undecanoate, |
| | | | | | | | Bayer; Testosterone undecanoate, Clarus; |
| | | | | | | | Testosterone, Acrux-1; Testosterone, Acrux-2; |
| | | | | | | | Testosterone, Alza; Testosterone, Antares; |
| | | | | | | | Testosterone, Auxilium; Testosterone, Auxilium-2; |
| | | | | | | | Testosterone, Cortecs; Testosterone, Lipocine; |
| | | | | | | | Testosterone, Pierre Fabre; Testosterone, Savient; |
| | | | | | | | Testosterone, TheraTech; Testosterone, Unimed; |
| | | | | | | | Testosterone, Watson, 2nd-generation; Testosterone, |
| | | | | | | | buccal, Columbia; Testosterone, vaginal, Columbia; |
| | | | | | | | Tibolo |
| ARSB | arylsulfatase B | Hippocampal | 18 | rs337847-? | Hippocampal | Fibrosis, general; Mucopolysaccharidosis VI | IBT-9402; Galsulfa |
| | | atrophy | | (NR) | atrophy | | |
| BCHE | butyrylcholinesterase | Heart failure | 16 | rs1523288-? | Heart failure | Alzheimer's disease; Dementia, Parkinson's | NP-0361; Bisnorcymserine; Butyrylcholinesterase, |
| | | | | (0.65) | | disease; Dementia, senile, general; Poisoning, | PharmAt; Butyrylcholinesterase, Shire; Rivastigmine |
| | | | | | | drug; Poisoning, organophosphate | tartrate; Rivastigmine tartrate, T |
| BMP2 | bone | Body mass | 70 | rs2145270-T | Body mass index | Bone disorder, general; Bone fracture healing; | BMP gene therapy, Wyeth; Dibotermin alfa; RhBMP-2, |
| | morphogenetic | index | | (0.65) | | Osteonecrosis; Osteoporosis; Regeneration, | Scil Technology-2; RhBPM-2/C |
| | protein 2 | | | | | bone, spinal fusion; Regeneration, cartilage | |
| BMP7 | bone | Response to | 5 | rs6127921-? | Response to | Arthritis, osteo; Bone fracture healing | Eptoterminal |
| | morphogenetic | citalopram | | (0.81), | citalopram | | |
| | protein 7 | treatment | | rs6127921-? | treatment | | |
| | | | | (0.82) | | | |
| CACNA1C | calcium channel, | Bipolar | 9 | rs1006737-A | Bipolar disorder | Anal fissure; Angina, general; Angina, unstable; | (S)-amlodipine, Emcure; BRL-32872; F-0401; NS-3601; |
| | voltage- | disorder and | | (0.36) | and major | Arrhythmia, general; Atherosclerosis; | PCA-50941; R-verapamil, Celltech; S-2150; SK-310; SL- |
| | dependent, L | major | | | depressive | Cardiomyopathy, ischaemic; Enuresis; Epilepsy, | 87.0495; Aliskiren + amlodipine + HCTZ, Novartis; |
| | type, alpha 1C | depressive | | | disorder | general; Fibrillation, atrial; Gastritis; | Aliskiren + amlodipine, Novartis; Amlodipine; |
| | subunit | disorder | | | | Haemorrhage, subarachnoid; Heart failure; | Amlodipine + benazepril; Amlodipine + irbesartan, |
| | | | | | | Hypertension, general; Incontinence, urinary; | Sanofi; Amlodipine + losartan + HCTZ, Merck; |
| | | | | | | Irritable bowel syndrome; Ischaemia, cerebral; | Amlodipine + losartan + hydrochlorothiazide, Hanmi; |
| | | | | | | Neurogenic bladder; Overactive bladder; | Amlodipine + losartan, HanAll; Amlodipine + |
| | | | | | | Peripheral vascular disease; Pollakisuria; Shock, | metoprolol XL, AZ; Amlodipine + rosuvastatin, HanAll; |
| | | | | | | traumatic; Tachycardia, supraventricular | Amlodipine + simvastatin, HanAll; Amlodipine + |
| | | | | | | | valsartan, Novartis; Amlodipine besilate + irbesartan, |
| | | | | | | | Dainippon; Amlodipine camsylate; Amlodipine |
| | | | | | | | maleate, SK; Amlodipine + HCTZ + valsartan, Nov; |
| | | | | | | | Amlodipine + losartan, Hanmi; Amlodipine, SUITAB; |
| | | | | | | | Aranidipine; Atenolol + nifedipine; Atorvastatin |
| | | | | | | | calcium + amlodipine; Azelnidipine; Benidipine |
| | | | | | | | hydrochloride; Bepridil; Bisaramil; Budralazine; |
| | | | | | | | Buflomedil; Candesartan cilexetil + amlodipine, |
| | | | | | | | Takeda; Cilnidipine; Cilnidipine + valsartan; Clevidipine |
| | | | | | | | butyrate; Delapril + manidipine, Chiesi; Diltiazem; |
| | | | | | | | Diltiazem SR, Ethypharm; Diltiazem hydrochloride, |
| | | | | | | | SLA; Diltiazem once-daily, Bago; Diltiazem once-daily, |
| | | | | | | | Biov; Diltiazem once-daily, Elan; Diltiazem once- |
| | | | | | | | daily, Biovail-2; Diltiazem twice-daily, Elan; Diltiazem, |
| | | | | | | | Alza; Diltiazem, Diffutab; Diltiazem, Douglas; |
| | | | | | | | Diltiazem, Edmond Pharma; Diltiazem, GEOMATRIX; |
| | | | | | | | Diltiazem, SURECAPS; Diltiazem, Watson; Diltiazem, |
| | | | | | | | Watson, PPDS; Diltiazem, Watson, SMHS; Diltiazem, |
| | | | | | | | once-daily, Mundiph; Diltiazem, once-daily, Sanofi; |
| | | | | | | | Diltiazem, once-daily, Verex; Efonidipine; Enalapril + |
| | | | | | | | diltiazem, Biovail; Enalapril + nitrendipine, Vita; |
| | | | | | | | Fantofarone; Felodipine; Felodipine + enalapril; |
| | | | | | | | Felodipine + metoprolol; Felodipine + ramipril; |
| | | | | | | | Gallopamil; Hydralazine + ISDN; Hydralazine + |
| | | | | | | | magnesium valproate; Neolpharma; Irbesartan; |
| CACNA1C | calcium channel, | Bipolar | 9 | rs1006737-A | Bipolar disorder | Anal fissure; Angina, general; Angina, unstable; | amlodipine, HanAll; Isradipine; Isradipine, OROS; |
| Continued | voltage- | disorder and | | (0.36) | and major | Arrhythmia, general; Atherosclerosis; | Lacidipine; Lemildipine; Lercanidipine; Lercanidipine + |
| | dependent, L | major | | | depressive | Cardiomyopathy, ischaemic; Enuresis; Epilepsy, | enalapril, Recordati; Lercanidipine + valsartan, LG Life |
| | type, alpha 1C | depressive | | | disorder | general; Fibrillation, atrial; Gastritis; | Sciences; Lercanidipine immediate release; |
| | subunit | disorder | | | | Haemorrhage, subarachnoid; Heart failure; | Lercanidipine, MeltDose; Manidipine; Mibefradil |
| | | | | | | Hypertension, general; Incontinence, urinary; | dihydrochloride; Nicardipine; Nifedipine; Nifedipine + |
| | | | | | | Irritable bowel syndrome; Ischaemia, cerebral; | candesartan, Bayer; Nifedipine once daily, Watson; |
| | | | | | | Neurogenic bladder; Overactive bladder; | Nifedipine twice-daily, Watson; Nifedipine, Alza; |
| | | | | | | Peripheral vascular disease; Pollakisuria; Shock, | Nifedipine, Elan; Nifedipine, EnSoTrol; Nifedipine, |
| | | | | | | traumatic; Tachycardia, supraventricular | Mundipharma; Nifedipine, Nippon Organon; |
| | | | | | | | Nifedipine, SURECAPS; Nifedipine, Siegfried; |
| | | | | | | | Nifedipine, TIMERx; Nilvadipine; Nimodipine; |
| | | | | | | | Nimodipine + endothelin antagonist, Edge; |
| | | | | | | | Nimodipine, Edge; Nisoldipine; Nisoldipine, |
| | | | | | | | SkyePharma; Nitrendipine; Olmesartan + amlodipine + |
| | | | | | | | HCTZ, Daiichi; Olmesartan + amlodipine, Daiichi |
| | | | | | | | Sankyo; Olmesartan + azelnidipine, Sankyo; Otilonium |
| | | | | | | | bromide; Phenytoin, Medisperse; Phenytoin, OROS; |
| | | | | | | | Phenytoin, Sirus; Pinaverium bromide; Pinaverium |
| | | | | | | | bromide + simethicone, Nycomed; Propiverine |
| | | | | | | | hydrochloride; S-amlodipine/telmisartan, Chong Kun |
| | | | | | | | Dang; Telmisartan + amlodipine, BI; Telmisartan + |
| | | | | | | | lacidipine, Glax; Tiapamil; Trandolapril + verapamil, |
| | | | | | | | Aven; Verapamil SR, Ethypharm; Verapamil, Eisai; |
| | | | | | | | Verapamil, Elan; Verapamil, Mylan; Verapamil, OROS, |
| | | | | | | | Alza; Verapamil, Orion-Farmos; Verapamil, Ver |
| CACNA1C | calcium channel, | Bipolar | 21 | rs1006737-A | Bipolar disorder | Anal fissure; Angina, general; Angina, unstable; | (S)-amlodipine, Emcure; BRL-32872; F-0401; NS-3601; |
| | voltage- | disorder | | (0.32) | | Arrhythmia, general; Atherosclerosis; | PCA-50941; R-verapamil, Celltech; S-2150; SK-310; SL- |
| | dependent, L | | | | | Cardiomyopathy, ischaemic; Enuresis; Epilepsy, | 87.0495; Aliskiren + amlodipine + HCTZ, Novartis; |
| | type, alpha 1C | | | | | general; Fibrillation, atrial; Gastritis; | Aliskiren + amlodipine, Novartis; Amlodipine; |
| | subunit | | | | | Haemorrhage, subarachnoid; Heart failure; | Amlodipine + benazepril; Amlodipine + irbesartan, |
| | | | | | | Hypertension, general; Incontinence, urinary; | Sanofi; Amlodipine + losartan + HCTZ, Merck; |
| | | | | | | Irritable bowel syndrome; Ischaemia, cerebral; | Amlodipine + losartan + hydrochlorothiazide, Hanmi; |
| | | | | | | Neurogenic bladder; Overactive bladder; | Amlodipine + losartan, HanAll; Amlodipine + |
| | | | | | | Peripheral vascular disease; Pollakisuria; Shock, | metoprolol XL, AZ; Amlodipine + rosuvastatin, HanAll; |
| | | | | | | traumatic; Tachycardia, supraventricular | Amlodipine + simvastatin, HanAll; Amlodipine + |
| | | | | | | | valsartan, Novartis; Amlodipine besilate + irbesartan, |
| | | | | | | | Dainippon; Amlodipine camsylate; Amlodipine |
| | | | | | | | maleate, SK; Amlodipine + HCTZ + valsartan, Nov; |
| | | | | | | | Amlodipine + losartan, Hanmi; Amlodipine, SUITAB; |
| | | | | | | | Aranidipine; Atenolol + nifedipine; Atorvastatin |
| | | | | | | | calcium + amlodipine; Azelnidipine; Benidipine |
| | | | | | | | hydrochloride; Bepridil; Bisaramil; Budralazine; |
| | | | | | | | Buflomedil; Candesartan cilexetil + amlodipine, |
| | | | | | | | Takeda; Cilnidipine; Cilnidipine + valsartan; Clevidipine |
| | | | | | | | butyrate; Delapril + manidipine, Chiesi; Diltiazem; |
| | | | | | | | Diltiazem SR, Ethypharm; Diltiazem hydrochloride, |
| | | | | | | | SLA; Diltiazem once-daily, Bago; Diltiazem once-daily, |
| | | | | | | | Biov; Diltiazem once-daily, Elan; Diltiazem once- |
| | | | | | | | daily, Biovail-2; Diltiazem twice-daily, Elan; Diltiazem, |
| | | | | | | | Alza; Diltiazem, Diffutab; Diltiazem, Douglas; |
| | | | | | | | Diltiazem, Edmond Pharma; Diltiazem, GEOMATRIX; |
| | | | | | | | Diltiazem, SURECAPS; Diltiazem, Watson; Diltiazem, |
| | | | | | | | Watson, PPDS; Diltiazem, Watson, SMHS; Diltiazem, |
| | | | | | | | once-daily, Mundiph; Diltiazem, once-daily, Sanofi; |
| | | | | | | | Diltiazem, once-daily, Verex; Efonidipine; Enalapril + |
| | | | | | | | diltiazem, Biovail; Enalapril + nitrendipine, Vita; |
| | | | | | | | Fantofarone; Felodipine; Felodipine + enalapril; |
| | | | | | | | Felodipine + metoprolol; Felodipine + ramipril; |
| | | | | | | | Gallopamil; Hydralazine + ISDN; Hydralazine + |
| | | | | | | | magnesium valproate; Neolpharma; Irbesartan; |
| CACNA1C | calcium channel, | Bipolar | 21 | rs1006737-A | Bipolar disorder | Anal fissure; Angina, general; Angina, unstable; | amlodipine, HanAll; Isradipine; Isradipine, OROS; |
| Continued | voltage- | disorder | | (0.32) | | Arrhythmia, general; Atherosclerosis; | Lacidipine; Lemildipine; Lercanidipine; Lercanidipine + |
| | dependent, L | | | | | Cardiomyopathy, ischaemic; Enuresis; Epilepsy, | enalapril, Recordati; Lercanidipine + valsartan, LG Life |
| | type, alpha 1C | | | | | general; Fibrillation, atrial; Gastritis; | Sciences; Lercanidipine immediate release; |
| | subunit | | | | | Haemorrhage, subarachnoid; Heart failure; | Lercanidipine, MeltDose; Manidipine; Mibefradil |
| | | | | | | Hypertension, general; Incontinence, urinary; | dihydrochloride; Nicardipine; Nifedipine; Nifedipine + |
| | | | | | | Irritable bowel syndrome; Ischaemia, cerebral; | candesartan, Bayer; Nifedipine once daily, Watson; |
| | | | | | | Neurogenic bladder; Overactive bladder; | Nifedipine twice-daily, Watson; Nifedipine, Alza; |
| | | | | | | Peripheral vascular disease; Pollakisuria; Shock, | Nifedipine, Elan; Nifedipine, EnSoTrol; Nifedipine, |
| | | | | | | traumatic; Tachycardia, supraventricular | Mundipharma; Nifedipine, Nippon Organon; |
| | | | | | | | Nifedipine, SURECAPS; Nifedipine, Siegfried; |
| | | | | | | | Nifedipine, TIMERx; Nilvadipine; Nimodipine; |
| | | | | | | | Nimodipine + endothelin antagonist, Edge; |
| | | | | | | | Nimodipine, Edge; Nisoldipine; Nisoldipine, |
| | | | | | | | SkyePharma; Nitrendipine; Olmesartan + amlodipine + |
| | | | | | | | HCTZ, Daiichi; Olmesartan + amlodipine, Daiichi |
| | | | | | | | Sankyo; Olmesartan + azelnidipine, Sankyo; Otilonium |
| | | | | | | | bromide; Phenytoin, Medisperse; Phenytoin, OROS; |
| | | | | | | | Phenytoin, Sirus; Pinaverium bromide; Pinaverium |
| | | | | | | | bromide + simethicone, Nycomed; Propiverine |
| | | | | | | | hydrochloride; S-amlodipine/telmisartan, Chong Kun |
| | | | | | | | Dang; Telmisartan + amlodipine, BI; Telmisartan + |
| | | | | | | | lacidipine, Glax; Tiapamil; Trandolapril + verapamil, |
| | | | | | | | Aven; Verapamil SR, Ethypharm; Verapamil, Eisai; |
| | | | | | | | Verapamil, Elan; Verapamil, Mylan; Verapamil, OROS, |
| | | | | | | | Alza; Verapamil, Orion-Farmos; Verapamil, Ver |
| CACNA2D1 | calcium channel, | Non-alcoholic | 16 | rs10954668-A | Non-alcoholic | Amyotrophic lateral sclerosis; Epilepsy, | NPRx-10; PD-299685; PF-4480682; Atagabalin; |
| | voltage- | fatty liver | | (0.33) | fatty liver | general; Epilepsy, partial (focal, local); | Gabapentin; Gabapentin + cobalamin + thiamine; |
| | dependent, alpha | disease | | | disease | Fibromyalgia; Generalized anxiety disorder; | Gabapentin AcuForm, Dep; Gabapentin enacarbil; |
| | 2/delta subunit 1 | histology | | | histology | Inflammation, urinary tract; Insomnia; | Gabapentin + oxybutynin, Dynogen; Gabapentin, |
| | | | | | | Menopausal symptoms, unspecified; Migraine | Medtronic; Imagabalin; Pregabalin; Pregabalin, |
| | | | | | | prophylaxis; Neuropathy, diabetic; Pain, | Protect; Pregabalin, controlled-relea |
| | | | | | | complex regional; Pain, general; Pain, | |
| | | | | | | musculoskeletal, general; Pain, neuropathic; | |
| | | | | | | Pain, post-herpetic; Pain, post-operative; | |
| | | | | | | Psychosis, bipolar; Restless legs syndrome; | |
| | | | | | | Sexual dysfunction, female; Social anxiety | |
| | | | | | | disorder | |
| CBLB | Cas-Br-M | Multiple | 59 | rs9657904-T | Multiple | Cancer, general | APN-401; APN-4 |
| | (murine) | sclerosis | | (0.826) | sclerosis | | |
| | ecotropic | | | | | | |
| | retroviral | | | | | | |
| | transforming | | | | | | |
| | sequence b | | | | | | |
| CCL2 | chemokine (C-C | Crohn's | 83 | rs3091315-A | Crohn's disease | Arthritis, rheumatoid; Asthma; Cancer, | ABN-912; Bindarit; Carlum |
| | motif) ligand 2 | disease | | (0.72) | | melanoma; Cancer, neuroendocrine, general; | |
| | | | | | | Cancer, ovarian; Cancer, prostate; Cancer, | |
| | | | | | | solid, general; Fibrosis, pulmonary, idiopathic; | |
| | | | | | | Hypertriglyceridaemia; Lupus nephritis; | |
| | | | | | | Nephropathy, diabetic; Psoriasis; Restenosis | |
| CCR1 | chemokine (C-C | Celiac disease | 65 | rs13098911-A | Celiac disease | Arthritis, rheumatoid; Chronic obstructive | AZD-4818; BMS-817399; CCX-354; MIP-1alpha, Wyeth; |
| | motif) receptor 1 | | | (0.10), | | pulmonary disease; Diabetes, Type 2; Lupus | NOX-E36; PS-0312 |
| | | | | rs6441961-A | | nephritis; Multiple sclerosis, general; | |
| | | | | (0.30) | | Nephropathy, diabetic; Radio/chemotherapy- | |
| | | | | | | induced injury, bone marrow, general | |
| CCR2 | chemokine (C-C | Celiac disease | 65 | rs13098911-A | Celiac disease | Arthritis, rheumatoid; Atherosclerosis; | AGI-1096; AZ-889; AZD-2423; AZD-5069; AZD-6942; |
| | motif) receptor 2 | | | (0.10) | | Bronchiectasis; Chronic obstructive pulmonary | CCL2 antagonists, Telik; CCR2 antagonist, OraPharma; |
| | | | | | | disease; Diabetes, Type 2; Fibrosis, liver; | CCR2b antagonist, CBT; CCX-140; EPX-102216; NIBR- |
| | | | | | | Hepatic dysfunction, general; Infection, | 6465; PF-4136309; TEI-8535; Cenicrivir |
| | | | | | | HIV/AIDS; Multiple sclerosis, general; | |
| | | | | | | Nephropathy, diabetic; Pain, general; Pain, | |
| | | | | | | musculoskeletal, general; Pain, neuropathic; | |
| | | | | | | Periodontitis; Restenosis; Transplant rejection, | |
| | | | | | | general | |
| CCR3 | chemokine (C-C | Celiac disease | 65 | rs13098911-A | Celiac disease | Asthma; Chronic obstructive pulmonary | 766994; ASM-8; AZD-1744; QAP-6 |
| | motif) receptor 3 | | | (0.10), | | disease; Rhinitis, allergic, general; Rhinitis, | |
| | | | | rs6441961-A | | general | |
| | | | | (0.30) | | | |
| CCR4 | chemokine (C-C | Celiac disease | 65 | rs13314993-C | Celiac disease | Asthma; Cancer, lymphoma, T-cell; Cancer, | CCR4-ZFN, T-cell, Sangamo; GSK-2239633; K-327; RS- |
| | motif) receptor 4 | | | (0.46) | | lymphoma, non-Hodgkin's; Eczema, atopic; | 1269; RS-1748; Mogamulizum |
| | | | | | | Infection, HIV/AIDS; Rhinitis, allergic, general | |
| CCR5 | chemokine (C-C | Celiac disease | 65 | rs13098911-A | Celiac disease | Alzheimer's disease; Amyotrophic lateral | 53926; AOP-RANTES; AZD-5672; CCR5 antagonists, |
| | motif) receptor 5 | | | (0.10) | | sclerosis; Arthritis, rheumatoid; Cachexia; | GlaxoSmithKline-2; CCR5 antagonists, Ono; |
| | | | | | | Cancer, general; Cancer, lymphoma, non- | CCR5mAb004; ESN-196; INCB-15050; NIBR-6465; PF- |
| | | | | | | Hodgkin's; Colitis, ulcerative; Cushing's disease; | 232798; PRO-140; RAP-101; RAP-160; RNAi HIV |
| | | | | | | Diabetes, Type 2; Herpetic keratitis; Infection, | therapy, stem cell, Benitec; RPI-MN; SB-728; SMA CCR- |
| | | | | | | HIV prophylaxis; Infection, HIV/AIDS; Infection, | 5/CXCR-4; SP-01A; TAK-220; VCH-286; Ancriviroc; |
| | | | | | | hepatitis-A virus; Infection, hepatitis-B virus; | Aplaviroc hydrochloride; Cenicriviroc; Dapivirine + |
| | | | | | | Infection, hepatitis-C virus; Infection, herpes | maraviroc, IPM; Maraviroc; Nifeviroc; Reticulose, |
| | | | | | | virus, unspecified; Infection, human papilloma | ADVR; Vicriviroc malea |
| | | | | | | virus; Infection, rabies; Multiple sclerosis, | |
| | | | | | | general; Psoriasis | |
| CCR9 | chemokine (C-C | Celiac disease | 65 | rs13098911-A | Celiac disease | Coeliac disease; Colitis, ulcerative; Crohn's | CCX-2 |
| | motif) receptor 9 | | | (0.10) | | disease; Inflammatory bowel disease, general | |
| CD19 | CD19 molecule | Crohn's | 83 | rs151181-G | Crohn's disease | Arthritis, rheumatoid; Cancer, haematological, | AFM-11; AFM-12; GBR-401; MDX-1342; MEDI-551; |
| | | disease | | (0.39) | | general; Cancer, leukaemia, acute lymphocytic; | Oncolysin B; SAR-3419; SGN-19A; XmAb-5574; |
| | | | | | | Cancer, leukaemia, chronic lymphocytic; | Blinatumom |
| | | | | | | Cancer, leukaemia, general; Cancer, | |
| | | | | | | lymphoma, B-cell; Cancer, lymphoma, general; | |
| | | | | | | Cancer, lymphoma, non-Hodgkin's; Cancer, | |
| | | | | | | myeloma; Scleroderma | |
| CD28 | CD28 molecule | Celiac disease | 65 | rs4675374-A | Celiac disease | Cancer, breast; Cancer, colorectal; Cancer, | AlloStim; Antisense 16064; CD40 inhibitors, Solvay; |
| | | | | (0.22) | | haematological, general; Cancer, leukaemia, | PRO-15 |
| | | | | | | general; Cancer, lung, non-small cell; Cancer, | |
| | | | | | | lymphoma, general; Cancer, melanoma; | |
| | | | | | | Cancer, myeloma; Cancer, ovarian; Cancer, | |
| | | | | | | prostate; Cancer, sarcoma, general; Cancer, | |
| | | | | | | solid, general; Psoriasis; Transplant rejection, | |
| | | | | | | general | |
| CD33 | CD33 molecule | Alzheimer's | 28 | rs3826656-? | Alzheimer's | Cancer, leukaemia, acute myelogenous; | AML therapy, Micromet; HuM195-Ac-225; IMGN-633; |
| | | disease | | (NR), | disease | Cancer, leukaemia, chronic myelogenous; | Gemtuzumab ozogamicin; Lintuzum |
| | | | | rs3865444-? | | Cancer, lung, small cell; Myelodysplastic | |
| | | | | (0.70) | | syndrome | |
| CD36 | CD36 molecule | Left | 3 | rs10499859-? | Left ventricular | Cancer, brain; Cancer, solid, general; Polycystic | ABT-898; PF-48568 |
| | (thrombospondin | ventricular | | (0.45) | mass | ovarian syndrome | |
| | receptor) | mass | | | | | |
| CD6 | CD6 molecule | Multiple | 59 | rs17824933-G | Multiple | Arthritis, rheumatoid; Cancer, lymphoma, non- | Anti-CD6 MAb, Bioc |
| | | sclerosis | | (0.25), | sclerosis | Hodgkin's; Psoriasis | |
| | | | | rs4939490-? | | | |
| | | | | (NR) | | | |
| CD80 | CD80 molecule | Celiac disease | 65 | rs11712165-C | Celiac disease | Ankylosing spondylitis; Arthritis, juvenile; | ASP-2408; Prostvac; RhuDex; Abatacept; Abatacept, |
| | | | | (0.39) | | Arthritis, psoriatic; Arthritis, rheumatoid; | BioXpress; Anti-B7 MAb, Solvay; Anti-B7 MAbs, Wyeth; |
| | | | | | | Asthma; Cancer, bladder; Cancer, lymphoma, | Fowlpox-TRICOM; Galixim |
| | | | | | | Hodgkin's; Cancer, lymphoma, non-Hodgkin's; | |
| | | | | | | Cancer, prostate; Colitis, ulcerative; Crohn's | |
| | | | | | | disease; Lupus nephritis; Psoriasis; Transplant | |
| | | | | | | rejection, bone marrow; Transplant rejection, | |
| | | | | | | general | |
| CD80 | CD80 molecule | Primary | 26 | rs2293370-G | Primary biliary | Ankylosing spondylitis; Arthritis, juvenile; | ASP-2408; Prostvac; RhuDex; Abatacept; Abatacept, |
| | | biliary | | (0.80) | cirrhosis | Arthritis, psoriatic; Arthritis, rheumatoid; | BioXpress; Anti-B7 MAb, Solvay; Anti-B7 MAbs, Wyeth; |
| | | cirrhosis | | | | Asthma; Cancer, bladder; Cancer, lymphoma, | Fowlpox-TRICOM; Galixim |
| | | | | | | Hodgkin's; Cancer, lymphoma, non-Hodgkin's; | |
| | | | | | | Cancer, prostate; Colitis, ulcerative; Crohn's | |
| | | | | | | disease; Lupus nephritis; Psoriasis; Transplant | |
| | | | | | | rejection, bone marrow; Transplant rejection, | |
| | | | | | | general | |
| CDK2 | cyclin-dependent | Type 1 | 50 | rs1701704-C | Type 1 diabetes | Cancer, adrenal; Cancer, brain; Cancer, breast; | AT-7519; CGP-60474; CGP-74514; CYC-065; PHA- |
| | kinase 2 | diabetes | | (0.35) | | Cancer, colorectal; Cancer, gastrointestinal, | 848125AC; SB-1317; Alvocidib; Dinaciclib; Seliciclib; |
| | | | | | | stomach; Cancer, general; Cancer, head and | Seliciclib, Alc |
| | | | | | | neck; Cancer, leukaemia, acute lymphocytic; | |
| | | | | | | Cancer, leukaemia, acute myelogenous; | |
| | | | | | | Cancer, leukaemia, chronic lymphocytic; | |
| | | | | | | Cancer, leukaemia, chronic myelogenous; | |
| | | | | | | Cancer, leukaemia, general; Cancer, liver; | |
| | | | | | | Cancer, lung, non-small cell; Cancer, | |
| | | | | | | lymphoma, B-cell; Cancer, lymphoma, general; | |
| | | | | | | Cancer, lymphoma, non-Hodgkin's; Cancer, | |
| | | | | | | melanoma; Cancer, mesothelioma; Cancer, | |
| | | | | | | myeloma; Cancer, nasopharyngeal; Cancer, | |
| | | | | | | neuroendocrine, general; Cancer, ovarian; | |
| | | | | | | Cancer, pancreatic; Cancer, prostate; Cancer, | |
| | | | | | | renal; Cancer, sarcoma, general; Cancer, solid, | |
| | | | | | | general; Cancer, thymoma; Glaucoma; | |
| | | | | | | Glomerulonephritis; Myelodysplastic syndrome | |
| CDK4 | cyclin-dependent | Celiac disease | 14 | rs10876993-? | Celiac disease | Cancer, brain; Cancer, breast; Cancer, | AT-7519; CDK4 gene, Celera; LEE-011; LY-2835219; P- |
| | kinase 4 | and | | (NR) | and Rheumatoid | colorectal; Cancer, gastrointestinal, stomach; | 1446-05; P-276-00; PD-0332991; PHA-848125AC; |
| | | Rheumatoid | | | arthritis | Cancer, general; Cancer, haematological, | Alvocid |
| | | arthritis | | | | general; Cancer, head and neck; Cancer, | |
| | | | | | | leukaemia, chronic lymphocytic; Cancer, | |
| | | | | | | leukaemia, chronic myelogenous; Cancer, | |
| | | | | | | leukaemia, general; Cancer, lung, non-small | |
| | | | | | | cell; Cancer, lymphoma, general; Cancer, | |
| | | | | | | lymphoma, non-Hodgkin's; Cancer, melanoma; | |
| | | | | | | Cancer, mesothelioma; Cancer, myeloma; | |
| | | | | | | Cancer, pancreatic; Cancer, prostate; Cancer, | |
| | | | | | | renal; Cancer, sarcoma, general; Cancer, solid, | |
| | | | | | | general; Cancer, squamous cell; Cancer, | |
| | | | | | | thymoma | |
| CDK6 | cyclin-dependent | Rheumatoid | 49 | rs42041-G | Rheumatoid | Cancer, breast; Cancer, gastrointestinal, | LEE-011; LY-2835219; PD-0332991; Alvocid |
| | kinase 6 | arthritis | | (0.24) | arthritis | stomach; Cancer, general; Cancer, head and | |
| | | | | | | neck; Cancer, leukaemia, chronic lymphocytic; | |
| | | | | | | Cancer, leukaemia, chronic myelogenous; | |
| | | | | | | Cancer, lung, non-small cell; Cancer, | |
| | | | | | | lymphoma, general; Cancer, lymphoma, non- | |
| | | | | | | Hodgkin's; Cancer, melanoma; Cancer, | |
| | | | | | | mesothelioma; Cancer, myeloma; Cancer, | |
| | | | | | | prostate; Cancer, renal; Cancer, sarcoma, | |
| | | | | | | general; Cancer, solid, general | |
| CDKN2A | cyclin-dependent | Glioma | 7 | rs2157719-? | Glioma | Cancer, general | CYC1 |
| | kinase inhibitor | | | (NR), | | | |
| | 2A (melanoma, | | | rs4977756-G | | | |
| | p16, inhibits | | | (0.60) | | | |
| | CDK4) | | | | | | |
| CDKN2A | cyclin-dependent | Melanoma | 4 | rs7023329-A | Melanoma | Cancer, general | CYC1 |
| | kinase inhibitor | | | (0.50) | | | |
| | 2A (melanoma, | | | | | | |
| | p16, inhibits | | | | | | |
| | CDK4) | | | | | | |
| CDKN2A | cyclin-dependent | Intracranial | 8 | rs1333040-T | Intracranial | Cancer, general | CYC1 |
| | kinase inhibitor | aneurysm | | (0.55), | aneurysm | | |
| | 2A (melanoma, | | | rs1333040-T | | | |
| | p16, inhibits | | | (0.56) | | | |
| | CDK4) | | | | | | |
| CDKN2A | cyclin-dependent | Abdominal | 2 | rs2383207-G | Abdominal | Cancer, general | CYC1 |
| | kinase inhibitor | aortic | | (0.49) | aortic aneurysm | | |
| | 2A (melanoma, | aneurysm | | | | | |
| | p16, inhibits | | | | | | |
| | CDK4) | | | | | | |
| CDKN2A | cyclin-dependent | Myocardial | 14 | rs10757278-G | Myocardial | Cancer, general | CYC1 |
| | kinase inhibitor | infarction | | (0.45), | infarction | | |
| | 2A (melanoma, | | | rs4977574-G | | | |
| | p16, inhibits | | | (0.56) | | | |
| | CDK4) | | | | | | |
| CDKN2A | cyclin-dependent | Type 2 | 61 | rs10811661-T | Type 2 diabetes | Cancer, general | CYC1 |
| | kinase inhibitor | diabetes | | (0.83), | | | |
| | 2A (melanoma, | | | rs10811661-T | | | |
| | p16, inhibits | | | (0.85), | | | |
| | CDK4) | | | rs10965250-G | | | |
| | | | | (NR), | | | |
| | | | | rs1333051-A | | | |
| | | | | (NR), | | | |
| | | | | rs2383208-A | | | |
| | | | | (0.55), | | | |
| | | | | rs564398-T | | | |
| | | | | (0.56), | | | |
| | | | | rs7020996-C | | | |
| | | | | (NR) | | | |
| CDKN2A | cyclin-dependent | Breast cancer | 37 | rs1011970-T | Breast cancer | Cancer, general | CYC1 |
| | kinase inhibitor | | | (0.17) | | | |
| | 2A (melanoma, | | | | | | |
| | p16, inhibits | | | | | | |
| | CDK4) | | | | | | |
| CDKN2A | cyclin-dependent | Coronary | 78 | 2-SNP | Coronary heart | Cancer, general | CYC1 |
| | kinase inhibitor | heart disease | | haplotype-4 | disease | | |
| | 2A (melanoma, | | | ((GG)), | | | |
| | p16, inhibits | | | rs1333049-C | | | |
| | CDK4) | | | (0.47), | | | |
| | | | | rs4977574-G | | | |
| | | | | (0.46) | | | |
| CETP | cholesteryl ester | Age-related | 16 | rs3764261-A | Age-related | Atherosclerosis; Hypercholesterolaemia; | 56419; ATH-03; CETP inhibitor, Johnson & Johnson; |
| | transfer protein, | macular | | (0.32), | macular | Hyperlipidaemia, general; Obesity | CETP inhibitors, Bayer-2; CETP inhibitors, Merck & Co; |
| | plasma | degeneration | | rs3764261-A | degeneration | | CETP vaccine, Celldex; CP-800569; DRL-17822; FM- |
| | | | | (0.33) | | | VP4; JTT-302; PF-3185043; Pharmaprojects No. 5691; |
| | | | | | | | Ro-51-32822; TA-8995; Anacetrapib; Atorvastatin + |
| | | | | | | | torcetrapib; Dalcetrapib; Dalcetrapib + atorvastatin, |
| | | | | | | | Roche; Evacetrapib; Torcetrap |
| CETP | cholesteryl ester | HDL | 6 | rs173539-C | HDL Cholesterol- | Atherosclerosis; Hypercholesterolaemia; | 56419; ATH-03; CETP inhibitor, Johnson & Johnson; |
| | transfer protein, | Cholesterol- | | (NR) | Triglycerides | Hyperlipidaemia, general; Obesity | CETP inhibitors, Bayer-2; CETP inhibitors, Merck & Co; |
| | plasma | Triglycerides | | | | | CETP vaccine, Celldex; CP-800569; DRL-17822; FM- |
| | | | | | | | VP4; JTT-302; PF-3185043; Pharmaprojects No. 5691; |
| | | | | | | | Ro-51-32822; TA-8995; Anacetrapib; Atorvastatin + |
| | | | | | | | torcetrapib; Dalcetrapib; Dalcetrapib + atorvastatin, |
| | | | | | | | Roche; Evacetrapib; Torcetrap |
| CETP | cholesteryl ester | Coronary | 78 | rs16965039-T | Coronary heart | Atherosclerosis; Hypercholesterolaemia; | 56419; ATH-03; CETP inhibitor, Johnson & Johnson; |
| | transfer protein, | heart disease | | (0.94) | disease | Hyperlipidaemia, general; Obesity | CETP inhibitors, Bayer-2; CETP inhibitors, Merck & Co; |
| | plasma | | | | | | CETP vaccine, Celldex; CP-800569; DRL-17822; FM- |
| | | | | | | | VP4; JTT-302; PF-3185043; Pharmaprojects No. 5691; |
| | | | | | | | Ro-51-32822; TA-8995; Anacetrapib; Atorvastatin + |
| | | | | | | | torcetrapib; Dalcetrapib; Dalcetrapib + atorvastatin, |
| | | | | | | | Roche; Evacetrapib; Torcetrap |
| CFH | complement | Age-related | 16 | rs1061170-? | Age-related | Haemolytic uraemic syndrome | Complement Factor H, L |
| | factor H | macular | | (NR), | macular | | |
| | | degeneration | | rs1061170-C | degeneration | | |
| | | | | (0.37), | | | |
| | | | | rs10737680-A | | | |
| | | | | (0.566), | | | |
| | | | | rs1329424-T | | | |
| | | | | (0.351), | | | |
| | | | | rs1329428-? | | | |
| | | | | (NR), | | | |
| | | | | rs1410996-? | | | |
| | | | | (NR), | | | |
| | | | | rs380390-C | | | |
| | | | | (0.70 | | | |
| | | | | (HapMap | | | |
| | | | | CEU)) | | | |
| CFH | complement | Nephropathy | 23 | rs6677604-? | Nephropathy | Haemolytic uraemic syndrome | Complement Factor H, L |
| | factor H | | | (0.77 (EA)) | | | |
| CFH | complement | Meningococcal | 2 | rs426736-? | Meningococcal | Haemolytic uraemic syndrome | Complement Factor H, L |
| | factor H | disease | | (0.84) | disease | | |
| CHEK2 | CHK2 checkpoint | Esophageal | 4 | rs738722-T | Esophageal | Cancer, bladder; Cancer, leukaemia, chronic | CBP-501; XL-8 |
| | homolog (S. pombe) | cancer and | | (0.25) | cancer and | lymphocytic; Cancer, lung, non-small cell; | |
| | | gastric cancer | | | gastric cancer | Cancer, lymphoma, general; Cancer, | |
| | | | | | | mesothelioma; Cancer, neuroendocrine, | |
| | | | | | | general; Cancer, oral; Cancer, ovarian; Cancer, | |
| | | | | | | solid, general | |
| CHRM3 | cholinergic | Hypertension | 22 | rs2820037-T | Hypertension | Alzheimer's disease; Asthma; Benign prostatic | 55284; 62380; ANAVEX-1007; ANAVEX-19-144; |
| | receptor, | | | (0.14) | | hyperplasia; Bronchitis, chronic; Cancer, | ANAVEX-2-73; ANAVEX-7-1037; GSK-961081; J- |
| | muscarinic 3 | | | | | colorectal; Cancer, lung, small cell; Cancer, | 104135; J-115311; LAS-190792; PSD-506; PT-010; |
| | | | | | | prostate; Cholangitis, unspecified; | Piloplex; SVT-47060; TRN-157; Aclidinium bromide; |
| | | | | | | Cholecystitis, unspecified; Cholelithiasis; | Aclidinium bromide + ICS; Aclidinium bromide + |
| | | | | | | Chronic obstructive pulmonary disease; Colitis, | formoterol; Alvameline; Atropine + pralidoxime |
| | | | | | | general; Cystic fibrosis; Depression, bipolar; | chloride; Cimetropium bromide; Clonidine + |
| | | | | | | Diabetes, Type 2; Dyskinesia, biliary; Enuresis; | oxybutynin; Darifenacin hydrobromide; Darotropium |
| | | | | | | Epilepsy, general; Gastritis; Gastro- | bromide; Denaverine hydrochloride; Formoterol |
| | | | | | | oesophageal reflux; Glaucoma; | fumarate + glycopyrrolate, Pearl; |
| | | | | | | Hypersalivation; Incontinence, urinary; | Gabapentin + oxybutynin, Dynogen; Glycopyrrolate + |
| | | | | | | Inflammation, urinary tract; Irritable bowel | indacaterol, Sosei; Glycopyrrolate, Chiesi; |
| | | | | | | syndrome; Ischaemia, cerebral; | Glycopyrrolate, Pearl; Glycopyrrolate, Shionogi; |
| | | | | | | Keratoconjunctivitis; Muscle spasm, general; | Glycopyrrolate, Sosei; Hyoscyamine sulfate; |
| | | | | | | Nausea and vomiting, general; Neurogenic | Hyoscyamine sulfate, InKine; Hyoscyamine sulfate, |
| | | | | | | bladder; Oesophagitis, reflux; Overactive | OraQuick; Imidafenacin; Imidafenacin, Kyorin (orally |
| | | | | | | bladder; Pain, musculoskeletal, general; Pain, | disintegrating); Ipratropium, Boehringer; Levalbuterol + |
| | | | | | | neuropathic; Pancreatitis; Pollakisuria; | ipratropium; Mebeverine + ispaghula; Olanzapine + |
| | | | | | | Postcholecystectomy syndrome; Psychosis, | pamoate acid; Olanzapine + zonisamide, Or; |
| | | | | | | bipolar; Psychosis, general; | Olanzapine, Alkermes; Olanzapine, RAIM; Olanzapine, |
| | | | | | | Radio/chemotherapy-induced injury, general; | Zydis; Olodaterol + tiotropium bromide; Oxybutynin |
| | | | | | | Schizophrenia; Ulcer, Gl, general; Ulcer, | gel, Antares; Oxybutynin ring, Barr; Oxybutynin, Alza; |
| | | | | | | duodenal; Ulcer, gastric; Xerophthalmia; | Oxybutynin, Auxilium; Oxybutynin, FemmePharma; |
| | | | | | | Xerostomia | Oxybutynin, Labopharm; Oxybutynin, Phoenix; |
| | | | | | | | Oxybutynin, TIMERx; Oxybutynin, TheraTech; |
| | | | | | | | Oxybutynin, gel, Watson; Pilocarpine + tolterodine, |
| | | | | | | | TheraVida; Pilocarpine, Cytokine; Pilocarpine, InSite; |
| | | | | | | | Pilocarpine, MGI; Pilocarpine, Pharmos; Propiverine |
| | | | | | | | hydrochloride; Salbutamol + ipratropium bromide; |
| | | | | | | | Scopolamine, Alza; Scopolamine, ETT; Secoverine; |
| CHRM3 | cholinergic | Hypertension | 22 | rs2820037-T | Hypertension | Alzheimer's disease; Asthma; Benign prostatic | Solifenacin; Solifenacin succinate; Solifenacin |
| continued | receptor, | | | (0.14) | | hyperplasia; Bronchitis, chronic; Cancer, | succinate + tamsulosin hydrochloride OCAS; |
| | muscarinic 3 | | | | | colorectal; Cancer, lung, small cell; Cancer, | Solifenacin succinate, ODT; Tamsulosin + tolterodine; |
| | | | | | | prostate; Cholangitis, unspecified; | Tarafenacin; Tiemonium metilsulfate; Tiemonium, |
| | | | | | | Cholecystitis, unspecified; Cholelithiasis; | Formenti; Tiotropium bromide; Tiotropium bromide + |
| | | | | | | Chronic obstructive pulmonary disease; Colitis, | formoterol fumarate + ciclesonide, Cipla; Tolterodine; |
| | | | | | | general; Cystic fibrosis; Depression, bipolar; | Tolterodine, extended-release; Trepibutone; |
| | | | | | | Diabetes, Type 2; Dyskinesia, biliary; Enuresis; | Trimebutine maleate + mosapride citrate, Samil; |
| | | | | | | Epilepsy, general; Gastritis; Gastro- | Trimebutine, Labopharm; Vamicami |
| | | | | | | oesophageal reflux; Glaucoma; | |
| | | | | | | Hypersalivation; Incontinence, urinary; | |
| | | | | | | Inflammation, urinary tract; Irritable bowel | |
| | | | | | | syndrome; Ischaemia, cerebral; | |
| | | | | | | Keratoconjunctivitis; Muscle spasm, general; | |
| | | | | | | Nausea and vomiting, general; Neurogenic | |
| | | | | | | bladder; Oesophagitis, reflux; Overactive | |
| | | | | | | bladder; Pain, musculoskeletal, general; Pain, | |
| | | | | | | neuropathic; Pancreatitis; Pollakisuria; | |
| | | | | | | Postcholecystectomy syndrome; Psychosis, | |
| | | | | | | bipolar; Psychosis, general; | |
| | | | | | | Radio/chemotherapy-induced injury, general; | |
| | | | | | | Schizophrenia; Ulcer, GI, general; Ulcer, | |
| | | | | | | duodenal; Ulcer, gastric; Xerophthalmia; | |
| | | | | | | Xerostomia | |
| CHRNA3 | cholinergic | Chronic | 7 | rs13180-? | Chronic | Addiction, alcohol; Addiction, nicotine | CP-6019 |
| | receptor, | obstructive | | (0.36), | obstructive | | |
| | nicotinic, alpha 3 | pulmonary | | rs8034191-C | pulmonary | | |
| | | disease | | (0.33) | disease | | |
| CHRNA3 | cholinergic | Lung cancer | 12 | rs8034191-? | Lung cancer | Addiction, alcohol; Addiction, nicotine | CP-6019 |
| | receptor, | | | (NR), | | | |
| | nicotinic, alpha 3 | | | rs8034191-C | | | |
| | | | | (0.34), | | | |
| | | | | rs8034191-G | | | |
| | | | | (NR) | | | |
| CHRNA3 | cholinergic | Lung | 8 | rs1051730-T | Lung | Addiction, alcohol; Addiction, nicotine | CP-6019 |
| | receptor, | adenocarcinoma | | (0.35) | adenocarcinoma | | |
| | nicotinic, alpha 3 | | | | | | |
| CHRNA4 | cholinergic | Chronic | 7 | rs8034191-C | Chronic | Addiction, nicotine; Alzheimer's disease; | ABT-560; AZD-1446; CP-601927; NS-9283; S-(+)- |
| | receptor, | obstructive | | (0.33) | obstructive | Attention deficit hyperactivity disorder; | mecamylamine HCI, Targacept; SUVN-90121; SUVN- |
| | nicotinic, alpha 4 | pulmonary | | | pulmonary | Cognitive disorder, unspecified; Dementia, | 911; TC-2696; TC-6499; Dianicline; Dianicline + |
| | | disease | | | disease | senile, general; Depression, general; | rimonabant; Ispronicline; Sofinicline; Tebanicline |
| | | | | | | Depression, major depressive disorder; | tosylate; Varenicline tartrate; Varenicline tartrate, T |
| | | | | | | Irritable bowel syndrome; Neuropathy, | |
| | | | | | | diabetic; Pain, general; Pain, neuropathic; Pain, | |
| | | | | | | post-herpetic; Pain, post-operative; | |
| | | | | | | Schizophrenia | |
| CHRNB4 | cholinergic | Lung cancer | 12 | rs8034191-C | Lung cancer | Addiction, alcohol; Addiction, nicotine | CP-6019 |
| | receptor, | | | (0.34), | | | |
| | nicotinic, beta 4 | | | rs8034191-G | | | |
| | | | | (NR) | | | |
| CLU | clusterin | Panic disorder | 10 | rs17466684-? | Panic disorder | Cancer, bladder; Cancer, breast; Cancer, lung, | AB-16B5; Custirs |
| | | | | (0.09) | | general; Cancer, lung, non-small cell; Cancer, | |
| | | | | | | ovarian; Cancer, prostate; Cancer, renal | |
| CLU | clusterin | Alzheimer's | 28 | 3-SNP | Alzheimer's | Cancer, bladder; Cancer, breast; Cancer, lung, | AB-16B5; Custirs |
| | | disease | | haplotype | disease | general; Cancer, lung, non-small cell; Cancer, | |
| | | | | (0.26), | | ovarian; Cancer, prostate; Cancer, renal | |
| | | | | rs11136000-? | | | |
| | | | | (0.60), | | | |
| | | | | rs1532278-? | | | |
| | | | | (0.64), | | | |
| | | | | rs569214-? | | | |
| | | | | (NR) | | | |
| COL1A1 | collagen, type I, | Breast cancer | 37 | rs2075555-? | Breast cancer | Cancer, general; Macular degeneration, age- | D |
| | alpha 1 | | | (NR) | | related, general | |
| COL4A1 | collagen, type IV, | Arterial | 1 | rs3742207-C | Arterial stiffness | Cancer, general; Macular degeneration, age- | D |
| | alpha 1 | stiffness | | (0.44) | | related, general | |
| COL4A1 | collagen, type IV, | Coronary | 78 | rs4773144-G | Coronary heart | Cancer, general; Macular degeneration, age- | D |
| | alpha 1 | heart disease | | (0.44) | disease | related, general | |
| CPS1 | carbamoyl- | Chronic | 67 | rs7422339-A | Chronic kidney | Hyperammonaemia | Carglumic acid, Orphan Euro |
| | phosphate | kidney | | (0.32) | disease | | |
| | synthase 1, | disease | | | | | |
| | mitochondrial | | | | | | |
| CR1 | complement | Alzheimer's | 28 | 2-SNP | Alzheimer's | Infection, Candida albicans; Infection, anthrax; | CDX-1135; Candida HP; ETI-204; ETI-211; TP |
| | component | disease | | haplotype | disease | Infection, anthrax prophylaxis; Infection, | |
| | (3b/4b) receptor | | | (0.18), | | staphylococcal; Ischaemia, cerebral; Macular | |
| | 1 (Knops blood | | | rs3818361-? | | degeneration, age-related, general; Renal | |
| | group) | | | (0.19), | | failure; Reperfusion injury; Respiratory distress | |
| | | | | rs6701713-A | | syndrome, adult; Surgery adjunct; Transplant | |
| | | | | (0.20) | | rejection, general | |
| CRHR1 | corticotropin | Bone mineral | 42 | rs9303521-T | Bone mineral | Anxiety, general; Arthritis, rheumatoid; | 586529; AAG-561; E-2508; MCI-028; NBI-27155; NBI- |
| | releasing | density | | (0.46) | density | Asthma; Cancer, brain; Depression, general; | 30545; NBI-30775; NBI-34041; NBI-35965; ONO- |
| | hormone | | | | | Depression, major depressive disorder; | 2333Ms; Pharmaprojects No. 5142; SSR-125543A; |
| | receptor 1 | | | | | Diagnosis, general; Epilepsy, general; Head | Corticorelin acetate; Pivagabine; Verucerfo |
| | | | | | | trauma; Inflammation, ocular; Insomnia; | |
| | | | | | | Irritable bowel syndrome; Ischaemia, cerebral; | |
| | | | | | | Oedema, cerebral; Post-traumatic stress | |
| | | | | | | disorder; Social anxiety disorder | |
| CRP | C-reactive | Lung cancer | 12 | rs2808630-G | Lung cancer | Arthritis, rheumatoid; Atherosclerosis; Cancer, | ISIS-353512; Pharmaprojects No. 51 |
| | protein, | | | (NR) | | myeloma; Fibrillation, atrial; Renal failure; | |
| | pentraxin-related | | | | | Respiratory distress syndrome, adult; | |
| | | | | | | Respiratory distress syndrome, infant; | |
| | | | | | | Transplant rejection, bone marrow | |
| CSF1 | colony | Paget's | 9 | rs10494112-G | Paget's disease | Arthritis, rheumatoid; Cancer, breast; Cancer, | ARRY-382; PD 03603 |
| | stimulating factor | disease | | (0.20), | | colorectal; Cancer, endometrial; Cancer, lung, | |
| | 1 (macrophage) | | | rs484959-? | | non-small cell; Cancer, ovarian; Cancer, | |
| | | | | (0.51) | | pancreatic; Cancer, prostate | |
| CSF2RA | colony | Schizophrenia | 33 | rs4129148-C | Schizophrenia | Anaemia, aplastic; Anaemia, | BBT-007; CSF-GM, Cangene; CSF-GM, LGLS; KB-002; |
| | stimulating factor | | | (NR) | | radio/chemotherapy-induced; Anaemia, renal | KB-003; MEN-11300; Mavrilimumab; Molgramostim; |
| | 2 receptor, alpha, | | | | | disease-induced; Arthritis, rheumatoid; | Molgramostim, Amoytop; Molgramostim, Probiomed; |
| | low-affinity | | | | | Asthma; Cancer, leukaemia, general; Cancer, | Molgramostim, Zenotech; Sargramostim; Talactoferrinal |
| | (granulocyte- | | | | | lung, non-small cell; Cancer, lymphoma, B-cell; | |
| | macrophage) | | | | | Cancer, lymphoma, non-Hodgkin's; Cancer, | |
| | | | | | | melanoma; Cancer, renal; Cancer, sarcoma, | |
| | | | | | | Kaposi's; Cancer, skin, general; Crohn's disease; | |
| | | | | | | Granulocytopenia; Infection, Escherichia coli | |
| | | | | | | prophylaxis; Infection, HIV/AIDS; Infection, | |
| | | | | | | unspecified; Neutropenia, general; Poisoning, | |
| | | | | | | radiation; Radio/chemotherapy-induced | |
| | | | | | | infection; Radio/chemotherapy-induced injury, | |
| | | | | | | bone marrow, general; Radio/chemotherapy- | |
| | | | | | | induced injury, bone marrow, neutropenia; | |
| | | | | | | Radio/chemotherapy-induced injury, general; | |
| | | | | | | Sepsis; Stem cell mobilization; | |
| | | | | | | Thrombocytopenic purpura; Ulcer, diabetic; | |
| | | | | | | Ulcer, venostasis; Vaccine adjunct; Wound | |
| | | | | | | healing | |
| CTLA4 | cytotoxic T- | Type 1 | 50 | rs3087243-? | Type 1 diabetes | Arthritis, rheumatoid; Cancer, bladder; Cancer, | ASP-2408; Belatacept; Ipilimumab; Tremelimum |
| | lymphocyte- | diabetes | | (NR), | | brain; Cancer, breast; Cancer, colorectal; | |
| | associated | | | rs3087243-A | | Cancer, gastrointestinal, stomach; Cancer, | |
| | protein 4 | | | (NR) | | genitourinary; Cancer, leukaemia, general; | |
| | | | | | | Cancer, liver; Cancer, lung, non-small cell; | |
| | | | | | | Cancer, lung, small cell; Cancer, lymphoma, B- | |
| | | | | | | cell; Cancer, lymphoma, non-Hodgkin's; | |
| | | | | | | Cancer, melanoma; Cancer, oesophageal; | |
| | | | | | | Cancer, ovarian; Cancer, pancreatic; Cancer, | |
| | | | | | | prostate; Cancer, renal; Infection, HIV/AIDS; | |
| | | | | | | Infection, hepatitis-C virus; Myelodysplastic | |
| | | | | | | syndrome; Transplant rejection, general | |
| CTLA4 | cytotoxic T- | Celiac disease | 65 | rs4675374-A | Celiac disease | Arthritis, rheumatoid; Cancer, bladder; Cancer, | ASP-2408; Belatacept; Ipilimumab; Tremelimum |
| | lymphocyte- | | | (0.22) | | brain; Cancer, breast; Cancer, colorectal; | |
| | associated | | | | | Cancer, gastrointestinal, stomach; Cancer, | |
| | protein 4 | | | | | genitourinary; Cancer, leukaemia, general; | |
| | | | | | | Cancer, liver; Cancer, lung, non-small cell; | |
| | | | | | | Cancer, lung, small cell; Cancer, lymphoma, B- | |
| | | | | | | cell; Cancer, lymphoma, non-Hodgkin's; | |
| | | | | | | Cancer, melanoma; Cancer, oesophageal; | |
| | | | | | | Cancer, ovarian; Cancer, pancreatic Cancer, | |
| | | | | | | prostate; Cancer, renal; Infection, HIV/AIDS; | |
| | | | | | | Infection, hepatitis-C virus; Myelodysplastic | |
| | | | | | | syndrome; Transplant rejection, general | |
| CTLA4 | cytotoxic T- | Alopecia | 11 | rs1024161-A | Alopecia areata | Arthritis, rheumatoid; Cancer, bladder; Cancer, | ASP-2408; Belatacept; Ipilimumab; Tremelimum |
| | lymphocyte- | areata | | (0.40) | | brain; Cancer, breast; Cancer, colorectal; | |
| | associated | | | | | Cancer, gastrointestinal, stomach; Cancer, | |
| | protein 4 | | | | | genitourinary; Cancer, leukaemia, general; | |
| | | | | | | Cancer, liver; Cancer, lung, non-small cell; | |
| | | | | | | Cancer, lung, small cell; Cancer, lymphoma, B- | |
| | | | | | | cell; Cancer, lymphoma, non-Hodgkin's; | |
| | | | | | | Cancer, melanoma; Cancer, oesophageal; | |
| | | | | | | Cancer, ovarian; Cancer, pancreatic; Cancer, | |
| | | | | | | prostate; Cancer, renal; Infection, HIV/AIDS; | |
| | | | | | | Infection, hepatitis-C virus; Myelodysplastic | |
| | | | | | | syndrome; Transplant rejection, general | |
| CTNNB1 | catenin | Bone mineral | 42 | rs87938-A | Bone mineral | Adenoma, colorectal; Cancer, colorectal; | CEQ-508; PRI-7 |
| | (cadherin- | density | | (0.45) | density | Cancer, pancreatic; Cancer, solid, general | |
| | associated | | | | | | |
| | protein), beta 1, | | | | | | |
| | 88 kDa | | | | | | |
| CTSH | cathepsin H | Type 1 | 50 | rs3825932-? | Type 1 diabetes | Dystrophy, Duchenne's muscular | Loxistat |
| | | diabetes | | (NR), | | | |
| | | | | rs3825932-T | | | |
| | | | | (0.68) | | | |
| CUBN | cubilin (intrinsic | Urinary | 3 | rs1801239-T | Urinary albumin | Amyotrophic lateral sclerosis; Anaemia, | Mecobalamin, Eisai; Vitamin B12, MDR |
| | factor-cobalamin | albumin | | (0.90) | excretion | pernicious; Anaemia, unspecified; Neuropathy, | |
| | receptor) | excretion | | | | diabetic; Neuropathy, unspecified; Nutrition | |
| CXCL12 | chemokine (C—X—C | Myocardial | 14 | rs1746048-C | Myocardial | Cancer, haematological, general; Cancer, solid, | NOX-A |
| | motif) ligand 12 | infarction | | (0.84) | infarction | general; Macular degeneration, age-related, | |
| | | | | | | general; Retinopathy, diabetic; Stem cell | |
| | | | | | | mobilization | |
| CXCL12 | chemokine (C—X—C | Coronary | 78 | rs1746048-C | Coronary heart | Cancer, haematological, general; Cancer, solid, | NOX-A |
| | motif) ligand 12 | heart disease | | (0.87), | disease | general; Macular degeneration, age-related, | |
| | | | | rs501120-T | | general; Retinopathy, diabetic; Stem cell | |
| | | | | (0.87) | | mobilization | |
| CYP17A1 | cytochrome | Parkinson's | 47 | rs17115100-G | Parkinson's | Cancer, breast; Cancer, prostate | ANG-3407; YM-116; Abiraterone acetate; |
| | P450, family 17, | disease | | (0.91) | disease | | Biphenylylmethylimidazole derivatives, Takeda; |
| | subfamily A, | | | | | | Galeterone; Orteron |
| | polypeptide 1 | | | | | | |
| CYP17A1 | cytochrome | Coronary | 78 | rs12413409-G | Coronary heart | Cancer, breast; Cancer, prostate | ANG-3407; YM-116; Abiraterone acetate; |
| | P450, family 17, | heart disease | | (0.89) | disease | | Biphenylylmethylimidazole derivatives, Takeda; |
| | subfamily A, | | | | | | Galeterone; Orteron |
| | polypeptide 1 | | | | | | |
| CYP1A1 | cytochrome | Coffee | 3 | rs2472297-T | Coffee | Cancer, solid, general | 5F-DF-2 |
| | P450, family 1, | consumption | | (0.265) | consumption | | |
| | subfamily A, | | | | | | |
| | polypeptide 1 | | | | | | |
| CYP1A1 | cytochrome | Diastolic | 24 | rs1378942-C | Diastolic blood | Cancer, solid, general | SF-DF-2 |
| | P450, family 1, | blood | | (0.36) | pressure | | |
| | subfamily A, | pressure | | | | | |
| | polypeptide 1 | | | | | | |
| CYP27B1 | cytochrome | Multiple | 59 | rs703842-A | Multiple | Hyperthyroidism | CTA-1 |
| | P450, family 27, | sclerosis | | (0.67) | sclerosis | | |
| | subfamily B, | | | | | | |
| | polypeptide 1 | | | | | | |
| CYP2C8 | cytochrome | Osteonecrosis | 1 | rs1934951-T | Osteonecrosis | Hypercholesterolaemia; Hyperlipidaemia, | Gemfibroz |
| | P450, family 2, | of the jaw | | (0.12) | of the jaw | general | |
| | subfamily C, | | | | | | |
| | polypeptide 8 | | | | | | |
| DCPS | decapping | Longevity | 23 | rs1695739-G | Longevity | Muscular atrophy, spinal | RG-30 |
| | enzyme, | | | (0.04) | | | |
| | scavenger | | | | | | |
| DDC | dopa | Acute | 13 | rs11978267-G | Acute | Parkinson's disease | AV-201; ProSavin; XP-21279 + carbidopa; Carbidopa + |
| | decarboxylase | lymphoblastic | | (0.27) | lymphoblastic | | levodopa CR, IMPAX; Carbidopa + levodopa ER, |
| | (aromatic L- | leukemia | | | leukemia | | Depomed; Carbidopa + levodopa GR, Intec; Carbidopa + |
| | amino acid | | | | | | levodopa, IMPAX; Carbidopa + levodopa, Penwest; |
| | decarboxylase) | | | | | | Carbidopa + levodopa, UCB; |
| | | | | | | | Carbidopa + entacapone + levodopa, Orion Pharma-2; |
| | | | | | | | Carbidopa + levodopa-1, Nouvel; Carbidopa, |
| | | | | | | | NeuroDerm; Entacapone + levodopa + carbidopa; |
| | | | | | | | Etilevodopa; Levodopa + benserazide, GEOMAT; |
| | | | | | | | Levodopa, Orion Pharma; Melevodopa; Melevodopa + |
| | | | | | | | carbidopa, |
| DDC | dopa | Malaria | 3 | rs1451375-? | Malaria | Parkinson's disease | AV-201; ProSavin; XP-21279 + carbidopa; Carbidopa + |
| | decarboxylase | | | (0.78) | | | levodopa CR, IMPAX; Carbidopa + levodopa ER, |
| | (aromatic L- | | | | | | Depomed; Carbidopa + levodopa GR, Intec; Carbidopa + |
| | amino acid | | | | | | levodopa, IMPAX; Carbidopa + levodopa, Penwest; |
| | decarboxylase) | | | | | | Carbidopa + levodopa, UCB; |
| | | | | | | | Carbidopa + entacapone + levodopa, Orion Pharma-2; |
| | | | | | | | Carbidopa + levodopa-1, Nouvel; Carbidopa, |
| | | | | | | | NeuroDerm; Entacapone + levodopa + carbidopa; |
| | | | | | | | Etilevodopa; Levodopa + benserazide, GEOMAT; |
| | | | | | | | Levodopa, Orion Pharma; Melevodopa; Melevodopa + |
| | | | | | | | carbidopa, |
| DHODH | dihydroorotate | Attention | 30 | rs16973500-C | Attention deficit | Arthritis, juvenile; Arthritis, psoriatic; Arthritis, | 4SC-302; FK-778; Genz-667348; LAS-186323; LAS- |
| | dehydrogenase | deficit | | (0.86) | hyperactivity | rheumatoid; Colitis, ulcerative; Crohn's | 187247; Leflunomide; Vidofludim |
| | | hyperactivity | | | disorder and | disease; Infection, HIV/AIDS; Infection, malaria; | |
| | | disorder and | | | conduct | Multiple sclerosis, general; Transplant | |
| | | conduct | | | disorder | rejection, general | |
| | | disorder | | | | | |
| DYRK1A | dual-specificity | HIV-1 | 1 | rs12483205-? | HIV-1 | Down's syndrome | DYRK1A inhibitors, NeuroNasce |
| | tyrosine-(Y)- | replication | | (NR) | replication | | |
| | phosphorylation | | | | | | |
| | regulated kinase | | | | | | |
| | 1A | | | | | | |
| DYRK1A | dual-specificity | Metabolic | 24 | rs2835810-? | Metabolic | Down's syndrome | DYRK1A inhibitors, NeuroNasce |
| | tyrosine-(Y)- | syndrome | | (0.37) | syndrome | | |
| | phosphorylation | | | | | | |
| | regulated kinase | | | | | | |
| | 1A | | | | | | |
| ENG | endoglin | Metabolic | 24 | rs7865146-A | Metabolic | Cancer, breast; Cancer, fallopian tube; Cancer, | TRC-1 |
| | | syndrome | | (0.37) | syndrome | ovarian; Cancer, peritoneal; Cancer, prostate; | |
| | | | | | | Cancer, solid, general; Macular degeneration, | |
| | | | | | | age-related, general | |
| ENPEP | glutamyl | Atrial | 2 | rs10033464-T | Atrial | Hypertension, general | QGC-0 |
| | aminopeptidase | fibrillation/atrial | | (0.08), | fibrillation/atrial | | |
| | (aminopeptidase | flutter | | rs2200733-T | flutter | | |
| | A) | | | (0.11) | | | |
| EPHA1 | EPH receptor A1 | Alzheimer's | 28 | rs11767557-? | Alzheimer's | Cancer, colorectal; Cancer, fallopian tube; | ENMD-20 |
| | | disease | | (0.81) | disease | Cancer, haematological, general; Cancer, | |
| | | | | | | leukaemia, acute myelogenous; Cancer, | |
| | | | | | | melanoma; Cancer, myeloma; Cancer, ovarian; | |
| | | | | | | Cancer, peritoneal; Cancer, renal | |
| ERBB3 | v-erb-b2 | Type 1 | 50 | rs11171739-C | Type 1 diabetes | Cancer, breast; Cancer, endometrial; Cancer, | AC-480; HM-781-36B; MEHD-7945A; MM-111; MM- |
| | erythroblastic | diabetes | | (0.42), | | fallopian tube; Cancer, general; Cancer, lung, | 121; RB-200h; U3-1287; RhErbB3 |
| | leukemia viral | | | rs1701704-C | | non-small cell; Cancer, ovarian; Cancer, | |
| | oncogene | | | (0.35), | | peritoneal; Cancer, solid, general | |
| | homolog 3 | | | rs2292239-? | | | |
| | (avian) | | | (NR), | | | |
| | | | | rs2292239-A | | | |
| | | | | (0.34), | | | |
| | | | | rs2292239-A | | | |
| | | | | (NR) | | | |
| ESR1 | estrogen | Alcohol | 8 | rs6902771-C | Alcohol | Acne; Alzheimer's disease; Cancer, breast; | 11096; 17AY-estradiol, Renovo; 3339; AZD-4992; |
| | receptor 1 | dependence | | (0.51) | dependence | Cancer, colorectal; Cancer, endometrial; | DHEA + acolbifene, Endoceutics; Diviseq; Femarelle; |
| | | | | | | Cancer, lung, non-small cell; Cancer, ovarian; | GTx-758; GTx-822; GW-5638; IP-1162; MK-6913; |
| | | | | | | Cancer, prostate; Contraceptive, female; | Nestorone + ethinylestradiol vaginal ring, Population |
| | | | | | | Cushing's disease; Depression, general; | Council; OTE-F, Pantarhei; P-09; RAD-1901; SERD |
| | | | | | | Eczema, seborrhoeic; Endometriosis; | programme, Aragon; SERM + toxin, SEEK; Synphase; |
| | | | | | | Gynaecomastia; Hormone replacement | TAS-108; VAL-201; Z-tamoxifen; Anti-estrogens, |
| | | | | | | therapy; Hypogonadism; Infarction, | Olema; Antiobesity SERM, Bionovo; Arzoxifene |
| | | | | | | myocardial; Infection, vaginal; Infertility, male; | hydrochloride; Bazedoxifene acetate; Bazedoxifene |
| | | | | | | Inflammation, vaginal; Insulin-related | acetate + Premarin; Chlormadinone acetate + ethinyl |
| | | | | | | metabolic syndrome; Lupus erythematosus, | estradiol; Contraceptive, BioSante; Cyproterone |
| | | | | | | systemic; Macular degeneration, age-related, | acetate + estradiol valerate, Bayer; Cyproterone |
| | | | | | | general; Menopausal symptoms, unspecified; | acetate + ethinylestradiol, Douglas; Desogestrel + |
| | | | | | | Multiple sclerosis, general; Multiple sclerosis, | estradiol, Akzo; Desogestrel + estrogen, tri; |
| | | | | | | relapsing-remitting; Obesity; Osteoporosis; | Desogestrel + ethinylestrad (1); |
| | | | | | | Parkinson's disease; Radio/chemotherapy- | Desogestrel + ethinylestrad (2); |
| | | | | | | induced injury, general; Seborrhoea; Sex- | Desogestrel + ethinylestrad (3); Desogestrel/ethinyl |
| | | | | | | chromosome abnormality, Turner's syndrome; | estradiol + ethinyl estradiol, Teva; Dienogest + |
| | | | | | | Sexual dysfunction, female; Sexual dysfunction, | estradiol valerate; Dienogest + estradiol valerate, |
| | | | | | | male; Sjogren's syndrome; Vaccine adjunct; | sequential, Bayer Schering; Dienogest + |
| | | | | | | Wound healing | ethinylestradiol; Droloxifene; Drospirenone + |
| | | | | | | | estradiol, Bayer Schering; Drospirenone + estradiol, |
| | | | | | | | Bayer-2; Drospirenone + ethinyl estradiol, Bayer; |
| | | | | | | | Drospirenone + ethinylestradiol + levomefolate |
| | | | | | | | calcium, Bayer-2; Drospirenone + ethinylestradiol + |
| | | | | | | | levomefolate calcium, Bayer-1; Drospirenone + |
| | | | | | | | ethinylestradiol + levomefolate calcium, Bayer-3; |
| | | | | | | | Drospirenone + ethinylestradiol, flexible dose, Bayer; |
| | | | | | | | Endoxifen; Estetrol; Estradiol (17AY), Noven; Estradiol |
| | | | | | | | (17AY), Solvay; Estradiol (patch), TheraTech; Estradiol + |
| | | | | | | | Nestorone, Antares; Estradiol + gestodene, Bayer; |
| | | | | | | | Estradiol + gestodene, Wyeth-2; Estradiol + |
| | | | | | | | levonorgestrel (patch), Bayer Schering; Estradiol + |
| | | | | | | | levonorgestrel, 3M; Estradiol + levonorgestrel, |
| | | | | | | | Jenderm; |
| ESR1 | estrogen | Alcohol | 8 | rs6902771-C | Alcohol | Acne; Alzheimer's disease; Cancer, breast; | Estradiol + levonorgestrel, Merck KGaA-1; Estradiol + |
| continued | receptor 1 | dependence | | (0.51) | dependence | Cancer, colorectal; Cancer, endometrial; | levonorgestrel-2, Merck KGaA; Estradiol + |
| | | | | | | Cancer, lung, non-small cell; Cancer, ovarian; | nomegestrol acetate; Estradiol + norethisterone; |
| | | | | | | Cancer, prostate; Contraceptive, female; | Estradiol + norethisterone, 4-day; Estradiol + |
| | | | | | | Cushing's disease; Depression, general; | norethisterone, Elan/Wyeth; Estradiol + |
| | | | | | | Eczema, seborrhoeic; Endometriosis; | norethisterone, Noven; Estradiol + norethisterone, |
| | | | | | | Gynaecomastia; Hormone replacement | Novo Nordisk; Estradiol + norethisterone, Pierre |
| | | | | | | therapy; Hypogonadism; Infarction, | Fabre; Estradiol + norethisterone, Sandoz; Estradiol + |
| | | | | | | myocardial; Infection, vaginal; Infertility, male; | progesterone, CAFET; Estradiol + progestogen, Acrux; |
| | | | | | | Inflammation, vaginal; Insulin-related | Estradiol + trimegestone; Estradiol 4-day, Elan; |
| | | | | | | metabolic syndrome; Lupus erythematosus, | Estradiol 7-day, Beta; Estradiol 7-day, Permatec; |
| | | | | | | systemic; Macular degeneration, age-related, | Estradiol acetate, Warner-1; Estradiol gel, Theramex; |
| | | | | | | general; Menopausal symptoms, unspecified; | Estradiol oral, Novo Nordisk; Estradiol patch, 3M |
| | | | | | | Multiple sclerosis, general; Multiple sclerosis, | Pharmaceuticals; Estradiol transdermal, Ortho; |
| | | | | | | relapsing-remitting; Obesity; Osteoporosis; | Estradiol vaginal, Novo Nordisk; Estradiol valerate + |
| | | | | | | Parkinson's disease; Radio/chemotherapy- | MPA, Orion-2; Estradiol valerate + levomefolate |
| | | | | | | induced injury, general; Seborrhoea; Sex- | calcium, Bayer; Estradiol valerate + |
| | | | | | | chromosome abnormality, Turner's syndrome; | medroxyprogesterone acetate, Orion; Estradiol |
| | | | | | | Sexual dysfunction, female; Sexual dysfunction, | valerate + medroxyprogesterone, Orion; Estradiol |
| | | | | | | male; Sjogren's syndrome; Vaccine adjunct; | valerate + norethisterone enanthate, Bayer; Estradiol |
| | | | | | | Wound healing | valerate, Orion; Estradiol(17AY) + norgestimate; |
| | | | | | | | Estradiol + dydrogesterone, Solv; Estradiol, 3M |
| | | | | | | | Pharmaceutical; Estradiol, 7-day patch, Rottapharm; |
| | | | | | | | Estradiol, Acrux; Estradiol, Alza; Estradiol, Antares; |
| | | | | | | | Estradiol, Bayer; Estradiol, Cilag; Estradiol, Elan; |
| | | | | | | | Estradiol, Endocon; Estradiol, MDRNA; Estradiol, Nitto |
| | | | | | | | Denko; Estradiol, Noven; Estradiol, Ortho-McNeil; |
| | | | | | | | Estradiol, Pharmacia; Estradiol, Pierre Fabre; Estradiol, |
| | | | | | | | Rottapharma; Estradiol, Servier; Estradiol, Solvay; |
| | | | | | | | Estradiol, Watson-2; Estradiol, pharmed; Estradiol, |
| | | | | | | | transdermal, Cipla; Estramustine phosphate sodium; |
| | | | | | | | Estriol, Adeona; Estrogen + progesterone, Agile-2; |
| | | | | | | | Estrogen + progestin, Cygnus; Estrogen + progestogen, |
| | | | | | | | Shire; Estrogen + progestogen, Alza; |
| ESR1 | estrogen | Alcohol | 8 | rs6902771-C | Alcohol | Acne; Alzheimer's disease; Cancer, breast; | Estrogen transdermal, 7-day, Merck KGaA; |
| continued | receptor 1 | dependence | | (0.51) | dependence | Cancer, colorectal; Cancer, endometrial; | Estrogen + progestin, 7 day, Cyg; Estrogen, Novavax; |
| | | | | | | Cancer, lung, non-small cell; Cancer, ovarian; | Estrogen, Noven; Estrogen, esterified, Solvay; |
| | | | | | | Cancer, prostate; Contraceptive, female; | Estrogens, conjugated, Barr; Ethinyl estradiol + |
| | | | | | | Cushing's disease; Depression, general; | norethindrone (low dose), Warner Chilcott-2; Ethinyl |
| | | | | | | Eczema, seborrhoeic; Endometriosis; | estradiol + norethindrone (low dose), Warner Chilcott- |
| | | | | | | Gynaecomastia; Hormone replacement | 3; Ethinyl estradiol sulfonate; Ethinyl estradiol, |
| | | | | | | therapy; Hypogonadism; Infarction, | Cassenne; Ethinyl estradiol, Savient; Ethinylestradiol + |
| | | | | | | myocardial; Infection, vaginal; Infertility, male; | TX-525, Th; Ethinylestradiol + levonorgestrel, Agile-1; |
| | | | | | | Inflammation, vaginal; Insulin-related | Ethinylestradiol + levonorgestrel, Barr; Ethinylestradiol + |
| | | | | | | metabolic syndrome; Lupus erythematosus, | levonorgestrel, Bayer; Ethinylestradiol + |
| | | | | | | systemic; Macular degeneration, age-related, | levonorgestrel, Bayer-2; Ethinylestradiol + |
| | | | | | | general; Menopausal symptoms, unspecified; | levonorgestrel, Gynetics; Ethinylestradiol + |
| | | | | | | Multiple sclerosis, general; Multiple sclerosis, | levonorgestrel, Teva; Ethinylestradiol + levonorgestrel, |
| | | | | | | relapsing-remitting; Obesity; Osteoporosis; | Wyeth; Ethinylestradiol + norelgestromin; |
| | | | | | | Parkinson's disease; Radio/chemotherapy- | Ethinylestradiol + norethindrone, Nobelpharma; |
| | | | | | | induced injury, general; Seborrhoea; Sex- | Ethinylestradiol + norethindrone, low-dose, |
| | | | | | | chromosome abnormality, Turner's syndrome; | Nobelpharma; Ethinylestradiol + norethisterone |
| | | | | | | Sexual dysfunction, female; Sexual dysfunction, | acetate, Warner; Ethinylestradiol + norethisterone, |
| | | | | | | male; Sjogren's syndrome; Vaccine adjunct; | Daiichi; Ethinylestradiol + norethisterone, Ortho; |
| | | | | | | Wound healing | Ethinylestradiol + etonogestrel; |
| | | | | | | | Ethinylestradiol + levonorges, Sc; Fispemifene; |
| | | | | | | | Fulvestrant; Gestodene + ethinylestradiol, Bayer-2; |
| | | | | | | | Gestodene + ethinylestradiol, Ba; Icaritin; Idoxifene; |
| | | | | | | | Lasofoxifene; Levormeloxifene; Medroxyprogesterone + |
| | | | | | | | Premarin; Medroxyprogesterone, depot; |
| | | | | | | | Miproxifene phosphate; Norethindrone + ethinyl |
| | | | | | | | estradiol (low dose), Warner Chilcott-1; |
| | | | | | | | Norethindrone acetate + ethinyl estradiol, Warner |
| | | | | | | | Chilcott-1; Norethindrone acetate + ethinyl estradiol, |
| | | | | | | | Warner Chilcott-2; Ormeloxifene; Ospemifene; |
| | | | | | | | Panomifene; Promestriene; Raloxifene hydrochloride; |
| | | | | | | | Synth conjugated estrogens, B; Tamoxifen; Tamoxifen, |
| | | | | | | | Douglas; Tamoxifen, oral liquid, Savient; Tibolone; |
| | | | | | | | Toremifene citrate; Trilostane; AY-estradiol prodrug |
| ESR1 | estrogen | Chronic | 9 | rs4869742-T | Chronic myeloid | Acne; Alzheimer's disease; Cancer, breast; | 11096; 17AY-estradiol, Renovo; 3339; AZD-4992; |
| | receptor 1 | myeloid | | (0.77) | leukemia | Cancer, colorectal; Cancer, endometrial; | DHEA + acolbifene, Endoceutics; Diviseq; Femarelle; |
| | | leukemia | | | | Cancer, lung, non-small cell; Cancer, ovarian; | GTx-758; GTx-822; GW-5638; IP-1162; MK-6913; |
| | | | | | | Cancer, prostate; Contraceptive, female; | Nestorone + ethinylestradiol vaginal ring, Population |
| | | | | | | Cushing's disease; Depression, general; | Council; OTE-F, Pantarhei; P-09; RAD-1901; SERD |
| | | | | | | Eczema, seborrhoeic; Endometriosis; | programme, Aragon; SERM + toxin, SEEK; Synphase; |
| | | | | | | Gynaecomastia; Hormone replacement | TAS-108; VAL-201; Z-tamoxifen; Anti-estrogens, |
| | | | | | | therapy; Hypogonadism; Infarction, | Olema; Antiobesity SERM, Bionovo; Arzoxifene |
| | | | | | | myocardial; Infection, vaginal; Infertility, male; | hydrochloride; Bazedoxifene acetate; Bazedoxifene |
| | | | | | | Inflammation, vaginal; Insulin-related | acetate + Premarin; Chlormadinone acetate + ethinyl |
| | | | | | | metabolic syndrome; Lupus erythematosus, | estradiol; Contraceptive, BioSante; Cyproterone |
| | | | | | | systemic; Macular degeneration, age-related, | acetate + estradiol valerate, Bayer; Cyproterone |
| | | | | | | general; Menopausal symptoms, unspecified; | acetate + ethinylestradiol, Douglas; Desogestrel + |
| | | | | | | Multiple sclerosis, general; Multiple sclerosis, | estradiol, Akzo; Desogestrel + estrogen, tri; |
| | | | | | | relapsing-remitting; Obesity; Osteoporosis; | Desogestrel + ethinylestrad (1); |
| | | | | | | Parkinson's disease; Radio/chemotherapy- | Desogestrel + ethinylestrad (2); |
| | | | | | | induced injury, general; Seborrhoea; Sex- | Desogestrel + ethinylestrad (3); Desogestrel/ethinyl |
| | | | | | | chromosome abnormality, Turner'syndrome; | estradiol + ethinyl estradiol, Teva; Dienogest + |
| | | | | | | Sexual dysfunction, female; Sexual dysfunction, | estradiol valerate; Dienogest + estradiol valerate, |
| | | | | | | male; Sjogren's syndrome; Vaccine adjunct; | sequential, Bayer Schering; Dienogest + |
| | | | | | | Wound healing | ethinylestradiol; Droloxifene; Drospirenone + |
| | | | | | | | estradiol, Bayer Schering; Drospirenone + estradiol, |
| | | | | | | | Bayer-2; Drospirenone + ethinyl estradiol, Bayer; |
| | | | | | | | Drospirenone + ethinylestradiol + levomefolate |
| | | | | | | | calcium, Bayer-2; Drospirenone + ethinylestradiol + |
| | | | | | | | levomefolate calcium, Bayer-1; Drospirenone + |
| | | | | | | | ethinylestradiol + levomefolate calcium, Bayer-3; |
| | | | | | | | Drospirenone + ethinylestradiol, flexible dose, Bayer; |
| | | | | | | | Endoxifen; Estetrol; Estradiol (17AY), Noven; Estradiol |
| | | | | | | | (17AY), Solvay; Estradiol (patch), TheraTech; Estradiol + |
| | | | | | | | Nestorone, Antares; Estradiol + gestodene, Bayer; |
| | | | | | | | Estradiol + gestodene, Wyeth-2; Estradiol + |
| | | | | | | | levonorgestrel (patch), Bayer Schering; Estradiol + |
| | | | | | | | levonorgestrel, 3M; Estradiol + levonorgestrel, |
| | | | | | | | Jenderm; Estradiol + levonorgestrel, Merck KGaA-1; |
| ESR1 | estrogen | Chronic | 9 | rs4869742-T | Chronic myeloid | Acne; Alzheimer's disease; Cancer, breast; | Estradiol + levonorgestrel-2, Merck KGaA; Estradiol + |
| continued | receptor 1 | myeloid | | (0.77) | leukemia | Cancer, colorectal; Cancer, endometrial; | nomegestrol acetate; Estradiol + norethisterone; |
| | | leukemia | | | | Cancer, lung, non-small cell; Cancer, ovarian; | Estradiol + norethisterone, 4-day; Estradiol + |
| | | | | | | Cancer, prostate; Contraceptive, female; | norethisterone, Elan/Wyeth; Estradiol + |
| | | | | | | Cushing's disease; Depression, general; | norethisterone, Noven; Estradiol + norethisterone, |
| | | | | | | Eczema, seborrhoeic; Endometriosis; | Novo Nordisk; Estradiol + norethisterone, Pierre |
| | | | | | | Gynaecomastia; Hormone replacement | Fabre; Estradiol + norethisterone, Sandoz; Estradiol + |
| | | | | | | therapy; Hypogonadism; Infarction, | progesterone, CAFET; Estradiol + progestogen, Acrux; |
| | | | | | | myocardial; Infection, vaginal; Infertility, male; | Estradiol + trimegestone; Estradiol 4-day, Elan; |
| | | | | | | Inflammation, vaginal; Insulin-related | Estradiol 7-day, Beta; Estradiol 7-day, Permatec; |
| | | | | | | metabolic syndrome; Lupus erythematosus, | Estradiol acetate, Warner-1; Estradiol gel, Theramex; |
| | | | | | | systemic; Macular degeneration, age-related, | Estradiol oral, Novo Nordisk; Estradiol patch, 3M |
| | | | | | | general; Menopausal symptoms, unspecified; | Pharmaceuticals; Estradiol transdermal, Ortho; |
| | | | | | | Multiple sclerosis, general; Multiple sclerosis, | Estradiol vaginal, Novo Nordisk; Estradiol valerate + |
| | | | | | | relapsing-remitting; Obesity; Osteoporosis; | MPA, Orion-2; Estradiol valerate + levomefolate |
| | | | | | | Parkinson's disease; Radio/chemotherapy- | calcium, Bayer; Estradiol valerate + |
| | | | | | | induced injury, general; Seborrhoea; Sex- | medroxyprogesterone acetate, Orion; Estradiol |
| | | | | | | chromosome abnormality, Turner's syndrome; | valerate + medroxyprogesterone, Orion; Estradiol |
| | | | | | | Sexual dysfunction, female; Sexual dysfunction, | valerate + norethisterone enanthate, Bayer; Estradiol |
| | | | | | | male; Sjogren's syndrome; Vaccine adjunct; | valerate, Orion; Estradiol(17AY) + norgestimate; |
| | | | | | | Wound healing | Estradiol + dydrogesterone, Solv; Estradiol, 3M |
| | | | | | | | Pharmaceutical; Estradiol, 7-day patch, Rottapharm; |
| | | | | | | | Estradiol, Acrux; Estradiol, Alza; Estradiol, Antares; |
| | | | | | | | Estradiol, Bayer; Estradiol, Cilag; Estradiol, Elan; |
| | | | | | | | Estradiol, Endocon; Estradiol, MDRNA; Estradiol, Nitto |
| | | | | | | | Denko; Estradiol, Noven; Estradiol, Ortho-McNeil; |
| | | | | | | | Estradiol, Pharmacia; Estradiol, Pierre Fabre; Estradiol, |
| | | | | | | | Rottapharma; Estradiol, Servier; Estradiol, Solvay; |
| | | | | | | | Estradiol, Watson-2; Estradiol, pharmed; Estradiol, |
| | | | | | | | transdermal, Cipla; Estramustine phosphate sodium; |
| | | | | | | | Estriol, Adeona; Estrogen + progesterone, Agile-2; |
| | | | | | | | Estrogen + progestin, Cygnus; Estrogen + progestogen, |
| | | | | | | | Shire; Estrogen + progestogen, Alza; Estrogen |
| | | | | | | | transdermal, 7-day, Merck KGaA; Estrogen + progestin, |
| | | | | | | | 7 day, Cyg; Estrogen, Novavax; Estrogen, Noven; |
| | | | | | | | Estrogen, esterified, Solvay; Estrogens, conjugated, |
| | | | | | | | Barr; Ethinyl estradiol + norethindrone (low dose), |
| ESR1 | estrogen | Chronic | 9 | rs4869742-T | Chronic myeloid | Acne; Alzheimer's disease; Cancer, breast; | Warner Chilcott-2; Ethinyl estradiol + norethindrone |
| continued | receptor 1 | myeloid | | (0.77) | leukemia | Cancer, colorectal; Cancer, endometrial; | (low dose), Warner Chilcott-3; Ethinyl estradiol |
| | | leukemia | | | | Cancer, lung, non-small cell; Cancer, ovarian; | sulfonate; Ethinyl estradiol, Cassenne; Ethinyl |
| | | | | | | Cancer, prostate; Contraceptive, female; | estradiol, Savient; Ethinylestradiol + TX-525, Th; |
| | | | | | | Cushing's disease; Depression, general; | Ethinylestradiol + levonorgestrel, Agile-1; |
| | | | | | | Eczema, seborrhoeic; Endometriosis; | Ethinylestradiol + levonorgestrel, Barr; Ethinylestradiol + |
| | | | | | | Gynaecomastia; Hormone replacement | levonorgestrel, Bayer; Ethinylestradiol + |
| | | | | | | therapy; Hypogonadism; Infarction, | levonorgestrel, Bayer-2; Ethinylestradiol + |
| | | | | | | myocardial; Infection, vaginal; Infertility, male; | levonorgestrel, Gynetics; Ethinylestradiol + |
| | | | | | | Inflammation, vaginal; Insulin-related | levonorgestrel, Teva; Ethinylestradiol + levonorgestrel, |
| | | | | | | metabolic syndrome; Lupus erythematosus, | Wyeth; Ethinylestradiol + norelgestromin; |
| | | | | | | systemic; Macular degeneration, age-related, | Ethinylestradiol + norethindrone, Nobelpharma; |
| | | | | | | general; Menopausal symptoms, unspecified; | Ethinylestradiol + norethindrone, low-dose, |
| | | | | | | Multiple sclerosis, general; Multiple sclerosis, | Nobelpharma; Ethinylestradiol + norethisterone |
| | | | | | | relapsing-remitting; Obesity; Osteoporosis; | acetate, Warner; Ethinylestradiol + norethisterone, |
| | | | | | | Parkinson's disease; Radio/chemotherapy- | Daiichi; Ethinylestradiol + norethisterone, Ortho; |
| | | | | | | induced injury, general; Seborrhoea; Sex- | Ethinylestradiol + etonogestrel; |
| | | | | | | chromosome abnormality, Turner's syndrome; | Ethinylestradiol + levonorges, Sc; Fispemifene; |
| | | | | | | Sexual dysfunction, female; Sexual dysfunction, | Fulvestrant; Gestodene + ethinylestradiol, Bayer-2; |
| | | | | | | male; Sjogren's syndrome; Vaccine adjunct; | Gestodene + ethinylestradiol, Ba; Icaritin; Idoxifene; |
| | | | | | | Wound healing | Lasofoxifene; Levormeloxifene; Medroxyprogesterone + |
| | | | | | | | Premarin; Medroxyprogesterone, depot; |
| | | | | | | | Miproxifene phosphate; Norethindrone + ethinyl |
| | | | | | | | estradiol (low dose), Warner Chilcott-1; |
| | | | | | | | Norethindrone acetate + ethinyl estradiol, Warner |
| | | | | | | | Chilcott-1; Norethindrone acetate + ethinyl estradiol, |
| | | | | | | | Warner Chilcott-2; Ormeloxifene; Ospemifene; |
| | | | | | | | Panomifene; Promestriene; Raloxifene hydrochloride; |
| | | | | | | | Synth conjugated estrogens, B; Tamoxifen; Tamoxifen, |
| | | | | | | | Douglas; Tamoxifen, oral liquid, Savient; Tibolone; |
| | | | | | | | Toremifene citrate; Trilostane; AY-estradiol prodrug |
| ESRRA | estrogen-related | Crohn's | 83 | rs694739-A | Crohn's disease | Cancer, brain; Cancer, colorectal; Cancer, lung, | SR-16388; Diaryl ether-based ligands, Johnson & Johns |
| | receptor alpha | disease | | (0.63) | | non-small cell; Cancer, myeloma; Cancer, | |
| | | | | | | prostate; Diabetes, general | |
| F11 | coagulation | Response to | 75 | rs13148903-G | Response to | Amyloidosis; Thrombosis, arterial; Thrombosis, | Factor XIa inhibitor, LG Life; ISIS-404071; ISIS-TTR |
| | factor XI | statin therapy | | (0.25) | statin therapy | general | |
| F12 | coagulation | Chronic | 67 | rs6420094-G | Chronic kidney | Ischaemia, cerebral; Thrombosis, arterial | Factor XIIa inhibitor, C |
| | factor XII | kidney | | (0.34) | disease | | |
| | (Hageman factor) | disease | | | | | |
| FAS | Fas (TNF receptor | Atrioventricular | 6 | rs1937332-G | Atrioventricular | Arthritis, osteo; Arthritis, rheumatoid; Cancer, | DE-098; F4509; Fas (delta) TM protein; MegaFasL; VB-1 |
| | superfamily, | conduction | | (0.47) | conduction | brain; Cancer, melanoma; Cancer, sarcoma, | |
| | member 6) | | | | | glial; Cancer, solid, general; Cancer, thyroid; | |
| | | | | | | Infarction, myocardial; Infection, HIV/AIDS; | |
| | | | | | | Reperfusion injury; Transplant rejection, bone | |
| | | | | | | marrow | |
| FASLG | Fas ligand (TNF | Celiac disease | 65 | rs859637-A | Celiac disease | Cancer, brain; Cancer, prostate; Infarction, | APG-101; Prostate cancer therapy, Sand |
| | superfamily, | | | (0.49) | | myocardial; Ischaemia, cerebral; Sepsis; | |
| | member 6) | | | | | Transplant rejection, bone marrow | |
| FCGR2B | Fc fragment of | Ulcerative | 96 | rs1801274-A | Ulcerative colitis | Arthritis, rheumatoid; Cancer, lymphoma, | SM-101; Anti-FcR antibody, SuppreM |
| | IgG, low affinity | colitis | | (0.51) | | general; Lupus erythematosus, systemic; | |
| | IIb, receptor | | | | | Multiple sclerosis, general; Psoriasis; | |
| | (CD32) | | | | | Thrombocytopenic purpura, idiopathic | |
| FDFT1 | farnesyl- | Non-alcoholic | 16 | rs2645424-A | Non-alcoholic | Cardiomyopathy, ischaemic; | BMS-188494; YM-S3601; Lapaquistat acetate; |
| | diphosphate | fatty liver | | (0.40) | fatty liver | Hypercholesterolaemia; Hyperlipidaemia, | Squalene synthase inhibitors, Daiichi Sank |
| | farnesyltransferase 1 | disease | | | disease | general; Hypertension, general | |
| | | histology | | | histology | | |
| FGF1 | fibroblast growth | Cardiac | 22 | rs152528-? | Cardiac | Peripheral vascular disease | 393 |
| | factor 1 (acidic) | hypertrophy | | (NR) | hypertrophy | | |
| FGF21 | fibroblast growth | Retinal | 18 | rs2287921-T | Retinal vascular | Diabetes, Type 2; Obesity | AZ-21; FGF-21 mimetics, Ambrx; LP-101 |
| | factor 21 | vascular | | (0.47) | caliber | | |
| | | caliber | | | | | |
| FGF4 | fibroblast growth | Breast cancer | 37 | rs614367-T | Breast cancer | Angina, general | Alferminogene tadenov |
| | factor 4 | | | (0.15) | | | |
| FLT1 | fms-related | Cognitive | 39 | rs17086609-? | Cognitive | Cancer, biliary; Cancer, bladder; Cancer, brain; | AL-3818; Angiozyme; BMS-690514; CEP-5214; CEP- |
| | tyrosine kinase 1 | performance | | (0.35) | performance | Cancer, breast; Cancer, cervical; Cancer, | 7055; KRN-633; MGCD-265; PRS-050; SSR-106462; |
| | (vascular | | | | | colorectal; Cancer, endometrial; Cancer, | STP-601; SU-14813; XL-999; Axitinib; Brivanib |
| | endothelial | | | | | fallopian tube; Cancer, gastrointestinal, | alaninate; Cediranib; Dovitinib lactate; Fruquintinib; |
| | growth | | | | | general; Cancer, gastrointestinal, stomach; | Icrucumab; Intedanib; Lenvatinib; Linifanib; |
| | factor/vascular | | | | | Cancer, gastrointestinal, stromal; Cancer, | Midostaurin; Motesanib diphosphate; Pazopanib |
| | permeability | | | | | general; Cancer, haematological, general; | hydrochloride; Pazopanib hydrochloride (ophthalmic); |
| | factor receptor) | | | | | Cancer, head and neck; Cancer, leukaemia, | Plitidepsin; Ponatinib; Sunitinib malate; Tivozanib; |
| | | | | | | acute lymphocytic; Cancer, leukaemia, acute | Vatalan |
| | | | | | | myelogenous; Cancer, leukaemia, chronic | |
| | | | | | | myelogenous; Cancer, leukaemia, general; | |
| | | | | | | Cancer, leukaemia, mast cell; Cancer, liver; | |
| | | | | | | Cancer, lung, general; Cancer, lung, non-small | |
| | | | | | | cell; Cancer, lung, small cell; Cancer, | |
| | | | | | | lymphoma, Hodgkin's; Cancer, lymphoma, T- | |
| | | | | | | cell; Cancer, lymphoma, general; Cancer, | |
| | | | | | | lymphoma, non-Hodgkin's; Cancer, melanoma; | |
| | | | | | | Cancer, mesothelioma; Cancer, myeloma; | |
| | | | | | | Cancer, nasopharyngeal; Cancer, | |
| | | | | | | neuroendocrine, carcinoid; Cancer, | |
| | | | | | | neuroendocrine, general; Cancer, | |
| | | | | | | neuroendocrine, pancreatic; Cancer, | |
| | | | | | | oesophageal; Cancer, ovarian; Cancer, | |
| | | | | | | pancreatic; Cancer, peritoneal; Cancer, | |
| | | | | | | prostate; Cancer, renal; Cancer, sarcoma, | |
| | | | | | | general; Cancer, sarcoma, leiomyo; Cancer, | |
| | | | | | | sarcoma, soft tissue; Cancer, sarcoma, | |
| | | | | | | synovial; Cancer, solid, general; Cancer, | |
| | | | | | | squamous cell; Cancer, thyroid; Cancer, | |
| | | | | | | urethral; Fibrosis, pulmonary, idiopathic; | |
| | | | | | | Herpetic keratitis; Idiopathic myelofibrosis; | |
| | | | | | | Macular degeneration, age-related, general; | |
| | | | | | | Macular degeneration, age-related, wet; | |
| | | | | | | Mastocytosis; Myelodysplastic syndrome; | |
| | | | | | | Oedema, macular, diabetic; Polycythaemia | |
| | | | | | | vera; Psoriasis; Retinopathy, diabetic | |
| FOLH1 | folate hydrolase | HDL | 37 | rs7395662-G | HDL cholesterol | Cancer, melanoma; Cancer, mesothelioma; | 131I-MIP-1375; 99mTc-MIP-1340; 99mTc-MIP-1404; |
| | (prostate-specific | cholesterol | | (0.61) | | Cancer, oesophageal; Cancer, prostate; Cancer, | 99mTc-MIP-1405; DCVax-prostate; EC-0652; EC-1069; |
| | membrane | | | | | renal; Cancer, solid, general; Diagnosis, cancer | GVX-3322; INO-5150; MIP-1375; MIP-220; MKC-1106- |
| | antigen) 1 | | | | | | PP; MLN-2704; PSMA ADC; PSMA gene vaccine, |
| | | | | | | | Progenics; Capromab pendetide; Prostate cancer |
| | | | | | | | immuno, Inovio; Prostate cancer vaccine, Cad |
| FOXO1 | forkhead box O1 | Central | 6 | rs2721051-G | Central corneal | Diabetes, Type 2 | AS-18428 |
| | | corneal | | (NR), | thickness | | |
| | | thickness | | rs2755237-A | | | |
| | | | | (NR) | | | |
| FSHR | follicle | Erectile | 23 | rs2268363-? | Erectile | Contraceptive, female; Infertility, female; | 62203; ADX-68692; FSH + LH, Ferring; FSH patch, |
| | stimulating | dysfunction | | (0.18) | dysfunction and | Infertility, male; Menopausal symptoms, | Pantec; FSH, 2nd-generation, Glycotope; FSH, LGLS; |
| | hormone | and prostate | | | prostate cancer | unspecified; Osteoporosis; Polycystic ovarian | FSH, recombinant, Watson/Itero; JR-041; LAPS-FSH; |
| | receptor | cancer | | | treatment | syndrome | Corifollitropin alfa; Follistatin, Arcarios; Follitropin alfa, |
| | | treatment | | | | | BioGeneriX; Follitropin alfa, Finox; Follitropin alfa, |
| | | | | | | | Merck Serono; Follitropin, Dong-A; Gonadotrophin, |
| | | | | | | | Merck Serono; Hyperglycosylated FSH, Merck Se; |
| | | | | | | | Lutropin alfa + follitropin alfa; RecFSH, Organon; |
| | | | | | | | Urofollitropin; Urofollitropin, Ferring; Urofollitropin, |
| | | | | | | | Merck Serono-2; Urofollitropin, Organ |
| GABBR1 | gamma- | Nasopharyngeal | 9 | rs29232-A | Nasopharyngeal | Addiction, alcohol; Anxiety, general; Autism; | ADX-71943; ALKS-29; AVE-1876; GABA-B receptor |
| | aminobutyric acid | carcinoma | | (0.46) | carcinoma | Cerebral palsy; Depression, general; Diabetic | agonists, Medisyn; Pharmaprojects No. 5525; SUN-09; |
| | (GABA) B | | | | | complication, general; Dyspepsia, non-ulcer; | Alprazolam extended-release; Arbaclofen placarbil; |
| | receptor, 1 | | | | | Epilepsy, general; Gastro-oesophageal reflux; | Arbaclofen, AGI Therapeutics; Arbaclofen, Seaside |
| | | | | | | Gastroparesis; Multiple sclerosis, general; | Therapeutics; Baclofen GR, Intec; Baclofen, IMPAX |
| | | | | | | Muscle spasm; Muscle spasm, general; Pain, | Laboratories; Baclofen, Sun Pharma; Baclofen, |
| | | | | | | musculoskeletal, arthritis; Pain, neuropathic; | intrathecal, Medtron; Lesogaber |
| | | | | | | Radio/chemotherapy-induced nausea and | |
| | | | | | | vomiting; Sex-chromosome abnormality, | |
| | | | | | | fragile X syndrome; Spasticity; Spinal cord | |
| | | | | | | injury | |
| GABRB3 | gamma- | Cognitive | 39 | rs8043440-? | Cognitive | Anxiety, general | XY-1029; XY-24 |
| | aminobutyric acid | performance | | (0.17) | performance | | |
| | (GABA) A | | | | | | |
| | receptor, beta 3 | | | | | | |
| GALC | galactosylceramidase | Crohn's | 83 | rs8005161-T | Crohn's disease | Leukodystrophy, globoid cell | Galactosylceramidase, Shire; Galactosylceramidase, |
| | | disease | | (0.12) | | | Zymen |
| GCK | glucokinase | Fasting | 6 | rs4607517-A | Fasting plasma | Benign prostatic hyperplasia; Diabetes, Type 1; | 53931; ARRY-403; AZD-1656; AZD-5658; AZD-6370; |
| | (hexokinase 4) | plasma | | (0.18) | glucose | Diabetes, Type 2; Diabetes, general; Obesity | AZD-6714; GKM-001; LY-2608204; MK-0599; PFE-GKA- |
| | | glucose | | | | | 2; PSN-010; RO-0281675; TAK-329; TTP-399; ZYGK-1; |
| | | | | | | | Glucokinase activator, Banyu; Glucokinase activator, |
| | | | | | | | Banyu-1; Glucokinase activator, Bristol-Myers Squibb; |
| | | | | | | | Glucokinase activator, LG Life Sciences; Glucokinase |
| | | | | | | | activator, Sanwa; Glucokinase activators, Novartis; |
| | | | | | | | Glucokinase activators, YuHan; Lonidamine, Thresho |
| GDNF | glial cell derived | Major | 34 | rs270545-G | Major | Amyotrophic lateral sclerosis; Parkinson's | AMT-090; ANG-2008; GDNF gene therapy, Copernicus; |
| | neurotrophic | depressive | | (0.69) | depressive | disease | Antiparkinsonian, Sanga |
| | factor | disorder | | | disorder | | |
| GHR | growth hormone | Systemic | 42 | rs979233-T | Systemic lupus | Acromegaly; Bone fracture healing; Cachexia; | ARX-201; CP-016; CP-024; IGF-1 + growth hormone, |
| | receptor | lupus | | (0.46) | erythematosus | Crohn's disease; Dwarfism; Dysplasia, | Ipsen; PEG-GH, PharmaEssentia; PEG-hGH, Phage |
| | | erythematosus | | | | ectodermal; Growth hormone deficiency; | Pharmaceuticals; YPEG-somatropin, Amoytop Biotech; |
| | | | | | | Infertility, female; Infertility, male; | Growth hormone, Bolder-2; Growth hormone, DelSite; |
| | | | | | | Lipodystrophy; Noonan syndrome; Prader-Willi | Human growth hormone, Daewoong; Human growth |
| | | | | | | Syndrome; Renal failure; Sex-chromosome | hormone, Delpor; Human growth hormone, PDA |
| | | | | | | abnormality, Turner's syndrome; Short-bowel | modi; Human growth hormone, Phage |
| | | | | | | syndrome | Pharmaceuticals; Human growth hormone, Versartis; |
| | | | | | | | Pegvisomant; Somatotropin, Genentech; Somatrem, |
| | | | | | | | Dong-A; Somatrem, Genentech; Somatropin mimetics, |
| | | | | | | | Celera; Somatropin prodrug, pegylated, Ascendis; |
| | | | | | | | Somatropin, Access; Somatropin, Amoytop Biotech; |
| | | | | | | | Somatropin, Bio Sidus-1; Somatropin, Dong-A; |
| | | | | | | | Somatropin, HanAll; Somatropin, LG Life Sciences; |
| | | | | | | | Somatropin, Lilly; Somatropin, Medusa, Flamel; |
| | | | | | | | Somatropin, Merck Serono; Somatropin, Novo |
| | | | | | | | Nordisk; Somatropin, OctoDEX; Somatropin, Pfizer; |
| | | | | | | | Somatropin, Pfizer, pegylated; Somatropin, ProLease; |
| | | | | | | | Somatropin, Prolor Biotech; Somatropin, SR, LG; |
| | | | | | | | Somatropin, Sandoz; Somatropin, Sanofi-Synthelabo; |
| | | | | | | | Somatropin, Savient; Somatropin, Soligenix; |
| | | | | | | | Somatropin, conjugate, Hanmi; Somatropin, pegylated |
| | | | | | | | long-acting, Novo Nordisk; |
| | | | | | | | Somatropin, Antares, needle-fr |
| GIP | gastric inhibitory | Coronary | 78 | rs46522-T | Coronary heart | Diabetes, Type 1 | EG- |
| | polypeptide | heart disease | | (0.53) | disease | | |
| GLG1 | golgi glycoprotein 1 | Breast cancer | 37 | 3-SNP | Breast cancer | Cancer, pancreatic | PAT-P |
| | | | | haplotype 1 | | | |
| | | | | (0.34) | | | |
| GPR39 | G protein- | Hypertension | 22 | 2-SNP | Hypertension | Diabetes, Type 2; Obesity | Antiobesity/antidiabetic, Anchor Therapeuti |
| | coupled receptor | | | haplotype-2 | | | |
| | 39 | | | ((AA)) | | | |
| GRIK1 | glutamate | Breast cancer | 37 | rs458685-? | Breast cancer | Neuropathy, diabetic; Pain, general; Pain, | LY-5456 |
| | receptor, | | | (NR) | | musculoskeletal, arthritis; Pain, neuropathic | |
| | ionotropic, | | | | | | |
| | kainate 1 | | | | | | |
| GRIK1 | glutamate | Response to | 75 | rs9305406-G | Response to | Neuropathy, diabetic; Pain, general; Pain, | LY-5456 |
| | receptor, | statin therapy | | (0.2) | statin therapy | musculoskeletal, arthritis; Pain, neuropathic | |
| | ionotropic, | | | | | | |
| | kainate 1 | | | | | | |
| GRIN2B | glutamate | Cognitive | 39 | rs2160519-? | Cognitive | Alzheimer's disease; Depression, general; | ED-1529; EVT-101; EVT-103; MK-0657; NMDA NR2B |
| | receptor, | performance | | (0.06) | performance | Depression, major depressive disorder; | subtype antagonists, AstraZeneca; NR2B antagonists, |
| | ionotropic, N- | | | | | Haemorrhage, subarachnoid; Huntington's | Merck; NR2B antagonists, NeurOp; NR2B antagonists, |
| | methyl D- | | | | | disease; Neuropathy, diabetic; Pain, general; | NeurOp-2; RG-1; Latrepirdine; Radiprod |
| | aspartate 2B | | | | | Pain, neuropathic; Pain, post-operative; | |
| | | | | | | Parkinson's disease | |
| GRM7 | glutamate | Panic disorder | 10 | rs3749380-? | Panic disorder | Addiction, alcohol; Depression, general; Post- | AMN-082; MGluR7 modulator, Add |
| | receptor, | | | (0.25) | | traumatic stress disorder | |
| | metabotropic 7 | | | | | | |
| GRM7 | glutamate | Major | 34 | rs9870680-T | Major | Addiction, alcohol; Depression, general; Post- | AMN-082; MGluR7 modulator, Add |
| | receptor, | depressive | | (0.44) | depressive | traumatic stress disorder | |
| | metabotropic 7 | disorder | | | disorder | | |
| HAVCR1 | hepatitis A virus | LDL | 42 | rs1501908-G | LDL cholesterol | Cancer, ovarian; Cancer, renal | CDX-0 |
| | cellular receptor 1 | cholesterol | | (0.37) | | | |
| HBB | hemoglobin, beta | Malaria | 3 | rs11036238-? | Malaria | Anaemia, sickle cell; Anaemia, unspecified; | AN-10; Aviheme; Pharmaprojects No. 4728; |
| | | | | (0.14) | | Thalassaemia | Pharmaprojects No. 4769; Pharmaprojects No. 5041; |
| | | | | | | | Thalagen; VX-366; Arginine butyrate; Globin gene |
| | | | | | | | therapy, Bluebird; Sickle cell therapy, Tapestry; |
| | | | | | | | Velares |
| HCRTR2 | hypocretin | HIV-1 control | 25 | rs9367630-? | HIV-1 control | Addiction, drug, unspecified; Anxiety, general; | 649868; CR-5542; Almorexant; Orexin 1/2 antagonist, |
| | (orexin) receptor 2 | | | (NR) | | Arthritis, rheumatoid; Chronic obstructive | Heptares; Orexin 1/2 inhibitors, Evotec; Orexin 2R |
| | | | | | | pulmonary disease; Insomnia; Sleep disorder, | modulator, Addex; Suvorexa |
| | | | | | | general | |
| HFE2 | hemochromatosis | Coronary | 78 | 2-SNP | Coronary heart | Anaemia, unspecified | Anaemia of inflammation therapy, Is |
| | type 2 (juvenile) | heart disease | | haplotype-1 | disease | | |
| | | | | ((CC)) | | | |
| HLA-B | major | HIV-1 control | 25 | rs2395029-? | HIV-1 control | Cancer, colorectal; Cancer, head and neck; | Velimogene aliplasm |
| | histocompatibility | | | (NR), | | Cancer, melanoma | |
| | complex, class I, B | | | rs2395029-G | | | |
| | | | | (0.032), | | | |
| | | | | rs2395029-G | | | |
| | | | | (0.048), | | | |
| | | | | rs2523590-C | | | |
| | | | | (0.164), | | | |
| | | | | rs2523608-? | | | |
| | | | | (0.37), | | | |
| | | | | rs2523608-G | | | |
| | | | | (0.326) | | | |
| HLA-B | major | Vitiligo | 25 | rs11966200-A | Vitiligo | Cancer, colorectal; Cancer, head and neck; | Velimogene aliplasm |
| | histocompatibility | | | (0.06) | | Cancer, melanoma | |
| | complex, class I, B | | | | | | |
| HLA-B | major | Drug-induced | 7 | rs2395029-? | Drug-induced | Cancer, colorectal; Cancer, head and neck; | Velimogene aliplasm |
| | histocompatibility | liver injury | | (0.05) | liver injury | Cancer, melanoma | |
| | complex, class I, B | | | | | | |
| HLA-B | major | Multiple | 59 | rs2523393-A | Multiple | Cancer, colorectal; Cancer, head and neck; | Velimogene aliplasm |
| | histocompatibility | sclerosis | | (0.59) | sclerosis | Cancer, melanoma | |
| | complex, class I, B | | | | | | |
| HMGCR | 3-hydroxy-3- | Body mass | 70 | rs2112347-T | Body mass index | Acute coronary syndrome; Alzheimer's disease; | 51445; 80/574 + atorvastatin; ASA + atorvastatin + |
| | methylglutaryl- | index | | (0.63) | | Atherosclerosis; Cancer, colorectal; Cognitive | ramipril + metoprolol ER, Zydus Cadila; BPL-003; BPL- |
| | CoA reductase | | | | | disorder, unspecified; Depression, general; | 004; CER-628; HBS-107; MT001; NCX-6560; NST-0037; |
| | | | | | | Diabetes, Type 2; Diabetic complication, | PPD-10558; Pharmaprojects No. 4662; Amlodipine + |
| | | | | | | general; Epilepsy, general; Head trauma; Heart | rosuvastatin, HanAll; Amlodipine + simvastatin, |
| | | | | | | failure; Hypercholesterolaemia; | HanAll; Atorvastatin; Atorvastatin + aspirin, Hanmi; |
| | | | | | | Hyperlipidaemia, general; | Atorvastatin + ezetimibe; Atorvastatin + fenofibrate |
| | | | | | | Hypertriglyceridaemia; Infarction, myocardial; | (micronized), Ethypharm; Atorvastatin + irbesartan, |
| | | | | | | Infection, influenia virus; Ischaemia, cerebral; | HanAll; Atorvastatin + torcetrapib; Atorvastatin |
| | | | | | | Osteoporosis; Pain, general; Renal failure; | calcium + amlodipine; Atorvastatin calcium IR + |
| | | | | | | Stenosis, aortic valve; Thrombosis, venous | losartan potassium DR, HanAll; Atorvastatin strontium, |
| | | | | | | | Hanmi; Benfluorex; Berivastatin; Cerivastatin sodium; |
| | | | | | | | Clopidogrel + simvastatin, Sanofi; Dalcetrapib + |
| | | | | | | | atorvastatin, Roche; Ezetimibe + simvastatin; |
| | | | | | | | Fenofibrate + pravastatin, SMB Laboratories; |
| | | | | | | | Fenofibrate + rosuvastatin-2; Fenofibrate + |
| | | | | | | | simvastatin, Abbott; Fenofibrate + pravastatin, |
| | | | | | | | Shionogi; Fluvastatin; Fluvastatin, extended release; |
| | | | | | | | Irbesartan + atorvastatin, Hanmi; |
| | | | | | | | Laropiprant + niacin + simvastatin; Losartan + |
| | | | | | | | simvastatin, HanAll; Lovastatin; Lovastatin, SCOT; |
| | | | | | | | Meglutol; Niacin + lovastatin, Kos; Niacin + |
| | | | | | | | simvastatin, Kos; Omacor + simvastatin, Sigma-Tau; |
| | | | | | | | Pitavastatin; Pravastatin; Pravastatin + ASA; |
| | | | | | | | Rosiglitazone maleate + simvastatin; Rosuvastatin + |
| | | | | | | | telmisartan, HanAll; Rosuvastatin calcium; |
| | | | | | | | Simvastatin; Simvastatin + ASA + ramipril, Ferrer; |
| | | | | | | | Simvastatin + triflusal stent; Simvastatin controlled |
| | | | | | | | release, Hamni; Sitagliptin + simvastat |
| HTR1A | 5- | Body mass | 70 | rs255414-A | Body mass index | Addiction, alcohol; Alzheimer's disease; | 1192090; ACN131; AL-8309A; AV-965; CM-2395; DSP- |
| | hydroxytryptamine | index | | (0.81) | | Amyotrophic lateral sclerosis; Anxiety, general; | 1053; DU-125530; ETI-0001; Lu-36-274; LuAA21004 |
| | (serotonin) | | | | | Attention deficit hyperactivity disorder; | (tablet); NPT-500; OPC-34712; Org-13011; PEL-576; |
| | receptor 1A | | | | | Autism; Depression, bipolar; Depression, | PF-217830; Pharmaprojects No. 5019; RGH-1756; |
| | | | | | | general; Depression, major depressive | RGH-1757; Rec 15-3079; SLV-314; SRA-444; TGFK- |
| | | | | | | disorder; Dyskinesia, levodopa-induced; | 08AA; Adoprazine; Alnespirone; Aripiprazole; |
| | | | | | | Dyspepsia, non-ulcer; Dysuria; Eczema, atopic; | Aripiprazole (once-weekly); Aripiprazole IM depot; |
| | | | | | | Epilepsy, general; Generalized anxiety disorder; | Aripiprazole, Alkermes; Aripiprazole, ODT; |
| | | | | | | Head trauma; Hypertension, general; | Aripiprazole, RapidFilm; Befiradol; Buspirone; |
| | | | | | | Incontinence, urinary; Infarction, cerebral; | Buspirone + melatonin, BrainCells; Buspirone, Acrux; |
| | | | | | | Irritable bowel syndrome; Ischaemia, cerebral; | Buspirone, Arcturus; Buspirone, Biovail; Eltoprazine; |
| | | | | | | Macular degeneration, age-related, general; | Eptapirone maleate; Espindolol; Flesinoxan; |
| | | | | | | Nausea and vomiting, general; Neuropathy, | Flibanserin; Gepirone; Ipsapirone; Levorphanol, |
| | | | | | | diabetic; Neuropathy, unspecified; Pain, | TheraQuest; Lurasidone hydrochloride; Naluzotan; |
| | | | | | | cancer; Pain, chemotherapy-induced; Pain, | Pardoprunox; Piclozotan; Quetiapine fumarate, SR; |
| | | | | | | general; Pain, neuropathic; Panic disorder; | Repinotan hydrochloride; Robalzotan tartrate hydrate; |
| | | | | | | Parkinson's disease; Post-traumatic stress | Sumanirole; Tandospirone; Urapidil; Vilazodone; |
| | | | | | | disorder; Psychosis, bipolar; Psychosis, general; | Xaliproden hydrochloride; Ziprasido |
| | | | | | | Restless legs syndrome; Schizophrenia; Sexual | |
| | | | | | | dysfunction, female; Tourette's syndrome | |
| HTR2A | 5- | Cardiac | 22 | rs1575891-? | Cardiac | Addiction, alcohol; Addiction, cocaine; Allergy, | 5-HT2A antagonists, Aventis-2; 53711; 55284; BVT- |
| | hydroxytryptamine | hypertrophy | | (NR) | hypertrophy | general; Alzheimer's disease; Anorexia | 28949; F-94116-CN; ITI-007; LY-2624803; MT210; N- |
| | (serotonin) | | | | | nervosa; Anxiety, general; Attention deficit | desmethylclozapine; OPC-34712; PF-217830; SL- |
| | receptor 2A | | | | | hyperactivity disorder; Autism; Depression, | 65.0472; UMN-02; YKP-1447; Antidepressants, Green |
| | | | | | | bipolar; Depression, general; Depression, | Cross; Aptazapine; Aripiprazole; Aripiprazole (once- |
| | | | | | | major depressive disorder; Dyskinesia, | weekly); Aripiprazole IM depot; Aripiprazole, |
| | | | | | | levodopa-induced; Fibromyalgia; Fibrosis, | Alkermes; Aripiprazole, ODT; Aripiprazole, RapidFilm; |
| | | | | | | general; Generalized anxiety disorder; | Asenapine maleate; Cyclobenzaprine, TONIX; |
| | | | | | | Glaucoma; Hypertension, general; | Deramciclane fumarate; Eplivanserin; Etoperidone; |
| | | | | | | Hypertension, pulmonary; Infarction, cerebral; | Fananserin; Flibanserin; Iferanserin, Ventrus; |
| | | | | | | Inflammatory bowel disease, general; | Iloperidone; Iloperidone, depot; Ketanserin; |
| | | | | | | Insomnia; Mental retardation; Migraine; | Lubazodone hydrochloride; Lurasidone hydrochloride; |
| | | | | | | Obsessive-compulsive disorder; Pain, general; | Mianserin; Nefazodone hydrochloride; Nelotanserin; |
| | | | | | | Pancreatitis; Panic disorder; Parkinson's | Olanzapine; Olanzapine + pamoate acid; Olanzapine + |
| | | | | | | disease; Peripheral vascular disease; Post- | zonisamide, Or; Olanzapine, Alkermes; Olanzapine, |
| | | | | | | traumatic stress disorder; Psychosis, bipolar; | RAIM; Olanzapine, Zydis; Paliperidone palmitate; |
| | | | | | | Psychosis, general; Schizophrenia; Sexual | Perospirone; Pimavanserin tartrate; Pipamperone + |
| | | | | | | dysfunction, female; Sleep disorder, general; | citalopram, PNB; Pipamperone + risperidone, |
| | | | | | | Thrombosis, arterial; Thrombosis, general; | PharmaNeuroBoost; Risperidone; Risperidone 4 wk |
| | | | | | | Tourette's syndrome; Transplant rejection, | long-acting injection (IM); Risperidone, Delpor; |
| | | | | | | general; Venous insufficiency | Risperidone, Medisorb; Risperidone, Nuvo Research; |
| | | | | | | | Risperidone, Quicklets; Sarpogrelate; Spiperone |
| | | | | | | | analogues; Temanogrel; Terguride, Ergonex; |
| | | | | | | | Volinanserin; Zicronapine; Ziprasido |
| HTR4 | 5- | Pulmonary | 13 | rs11168048-T | Asthma/Severe | Alzheimer's disease; Constipation; Diabetic | 5-ht4 partial agonist, pfizer; 5-ht4 partial agonists, |
| | hydroxytryptamine | function | | (0.58), | asthma/cough | complication, general; Dyspepsia; Dyspepsia, | concert; Cinitapride; Da-6650; Gi hypermotility |
| | (serotonin) | | | rs3995090-C | | non-ulcer; Gastritis; Gastro-oesophageal reflux; | disorders ther, movetis; Indisetron; Levosulpiride; M- |
| | receptor 4 | | | (0.41) | | Gastroparesis; Heart failure; Ileus; Irritable | 0003; M-0004; M-0014; Metoclopramide + lysine |
| | | | | | | bowel syndrome; Motility dysfunction, GI, | acetyl; Metoclopramide, diffucap; Metoclopramide, |
| | | | | | | general; Nausea and vomiting, general; Nausea | mdrna; Metoclopramide, nasal spray, evoke; |
| | | | | | | Radio/chemotherapy-induced nausea and | Metoclopramide-zydis, salix; Mosapride; Naronapride; |
| | | | | | | vomiting | Omeprazole + cinitapride er, zydus; Pantoprazole + |
| | | | | | | | cinitapride er, zydus; Prucalopride; Prx-03140; Rq-10; |
| | | | | | | | Rq-9; Ser-103; Suvn-91052; Tegaserod maleate; |
| | | | | | | | Velusetrag; Ykp-10811 |
| ICAM3 | intercellular | Crohn's | 83 | rs12720356-G | Crohn's disease | Cancer, leukaemia, acute myelogenous; | IC |
| | adhesion | disease | | (0.08) | | | |
| | molecule 3 | | | | | | |
| ICOS | inducible T-cell | Celiac disease | 65 | rs4675374-A | Celiac disease | Lupus erythematosus, systemic | MEDI-570 |
| | co-stimulator | | | (0.22) | | | |
| ICOS | inducible T-cell | Alopecia | 11 | rs1024161-A | Alopecia areata | Lupus erythematosus, systemic | MEDI-5 |
| | co-stimulator | areata | | (0.40) | | | |
| ICOSLG | inducible T-cell | Celiac disease | 65 | rs4819388-? | Celiac disease | Lupus erythematosus, systemic | AMG-5 |
| | co-stimulator | | | (0.72) | | | |
| | ligand | | | | | | |
| ICOSLG | inducible T-cell | Crohn's | 83 | rs762421-G | Crohn's disease | Lupus erythematosus, systemic | AMG-5 |
| | co-stimulator | disease | | (0.39) | | | |
| | ligand | | | | | | |
| ICOSLG | inducible T-cell | Ulcerative | 96 | rs2838519-G | Ulcerative colitis | Lupus erythematosus, systemic | AMG-5 |
| | co-stimulator | colitis | | (0.39) | | | |
| | ligand | | | | | | |
| IDS | iduronate 2- | Cognitive test | 33 | rs530501-? | Cognitive test | Mucopolysaccharidosis II | JR-032; Idursulfa |
| | sulfatase | performance | | (NR) | performance | | |
| IFNG | interferon, | Ulcerative | 96 | rs1558744-A | Ulcerative colitis | Arthritis, rheumatoid; Cachexia; Cancer, basal | AMG-811; Ad-IFNgamma; Anti-IFN-gamma MAb, |
| | gamma | colitis | | (0.38), | | cell; Cancer, liver; Cancer, lymphoma, B-cell; | Solvay; Fontolizumab; Interferon gamma, A5 |
| | | | | rs1558744-A | | Cancer, lymphoma, T-cell; Cancer, melanoma; | Laboratori |
| | | | | (NR), | | Cancer, renal; Crohn's disease; | |
| | | | | rs7134599-A | | Glomerulonephritis; Lupus erythematosus, | |
| | | | | (0.39) | | cutaneous; Lupus erythematosus, systemic; | |
| | | | | | | Lupus nephritis; Multiple sclerosis, general; | |
| | | | | | | Psoriasis | |
| IGF1 | insulin-like | Fasting | 2 | rs35767-G | Fasting insulin- | Acromegaly; Amyotrophic lateral sclerosis; | ATL-1103; CERE-135; IGF-1 neurological therapy, |
| | growth factor 1 | insulin- | | (0.85) | related traits | Autism; Ischaemia, cerebral; Macular | Braasch; Pharmaprojects No. 58 |
| | (somatomedin C) | related traits | | | | degeneration, age-related, wet; Retinopathy, | |
| | | | | | | diabetic | |
| IGF1 | insulin-like | Fasting | 18 | rs35767-G | Fasting glucose- | Acromegaly; Amyotrophic lateral sclerosis; | ATL-1103; CERE-135; IGF-1 neurological therapy, |
| | growth factor 1 | glucose- | | (0.85) | related traits | Autism; Ischaemia, cerebral; Macular | Braasch; Pharmaprojects No. 58 |
| | (somatomedin C) | related traits | | | | degeneration, age-related, wet; Retinopathy, | |
| | | | | | | diabetic | |
| IL10 | interleukin 10 | Type 1 | 50 | rs3024505-? | Type 1 diabetes | Arthritis, rheumatoid; Infection, Ebola virus; | 54197; EG-10; HMPL-011; Autoimmune therapy, Epidy |
| | | diabetes | | (0.83) | | Infection, Marburg virus; Inflammatory bowel | |
| | | | | | | disease, general; Psoriasis | |
| IL10 | interleukin 10 | Crohn's | 83 | rs3024505-T | Crohn's disease | Arthritis, rheumatoid; Infection, Ebola virus; | 54197; EG-10; HMPL-011; Autoimmune therapy, Epidy |
| | | disease | | (0.16) | | Infection, Marburg virus; Inflammatory bowel | |
| | | | | | | disease, general; Psoriasis | |
| IL10 | interleukin 10 | Behcet's | 3 | rs1518111-? | Behcet's disease | Arthritis, rheumatoid; Infection, Ebola virus; | 54197; EG-10; HMPL-011; Autoimmune therapy, Epidy |
| | | disease | | (NR), | | Infection, Marburg virus; Inflammatory bowel | |
| | | | | rs1800871-T | | disease, general; Psoriasis | |
| | | | | (0.31) | | | |
| IL12A | interleukin 12A | Celiac disease | 65 | rs17810546-G | Celiac disease | Arthritis, rheumatoid; Cancer, brain; Cancer, | EGEN-001; IL-12 + IFN-alpha gene ther, Ino; |
| | (natural killer cell | | | (0.13), | | colorectal; Cancer, fallopian tube; Cancer, head | Briakinumab; Cancer gene therapy, Intrexon; |
| | stimulatory factor | | | rs17810546-G | | and neck; Cancer, melanoma; Cancer, ovarian; | Hepatitis-B DNA vaccine, Genexine; Interleukin-12, |
| | 1, cytotoxic | | | (NR) | | Cancer, pancreatic; Cancer, peritoneal; Crohn's | Neumedicin |
| | lymphocyte | | | | | disease; Infection, hepatitis-B virus; Multiple | |
| | maturation factor | | | | | sclerosis, general; Poisoning, radiation; | |
| | 1, p35) | | | | | Psoriasis | |
| IL12A | interleukin 12A | Primary | 26 | rs485499-T | Primary biliary | Arthritis, rheumatoid; Cancer, brain; Cancer, | EGEN-001; IL-12 + IFN-alpha gene ther, Ino; |
| | (natural killer cell | biliary | | (0.57), | cirrhosis | colorectal; Cancer, fallopian tube; Cancer, head | Briakinumab; Cancer gene therapy, Intrexon; |
| | stimulatory factor | cirrhosis | | rs6441286-G | | and neck; Cancer, melanoma; Cancer, ovarian; | Hepatitis-B DNA vaccine, Genexine; Interleukin-12, |
| | 1, cytotoxic | | | (0.39), | | Cancer, pancreatic; Cancer, peritoneal; Crohn's | Neumedicin |
| | lymphocyte | | | rs6441286-G | | disease; Infection, hepatitis-B virus; Multiple | |
| | maturation factor | | | (0.394) | | sclerosis, general; Poisoning, radiation; | |
| | 1, p35) | | | | | Psoriasis | |
| IL12B | interleukin 12B | Crohn's | 83 | rs10045431-? | Crohn's disease | Arthritis, psoriatic; Arthritis, rheumatoid; | CEP-37248; CI-201; EGEN-001; FM-202; GX-P04; IL-12 + |
| | (natural killer cell | disease | | (NR), | | Atherosclerosis; Cancer, brain; Cancer, | IFN-alpha gene ther, Ino; Briakinumab; Cancer gene |
| | stimulatory factor | | | rs10045431-C | | colorectal; Cancer, fallopian tube; Cancer, head | therapy, Intrexon; Hepatitis-B DNA vaccine, Genexine; |
| | 2, cytotoxic | | | (0.71), | | and neck; Cancer, melanoma; Cancer, ovarian; | Interleukin-12, Neumedicines; Ustekinumab; |
| | lymphocyte | | | rs6887695-? | | Cancer, pancreatic; Cancer, peritoneal; | Ustekinumab, BioXpre |
| | maturation factor | | | (0.32) | | Cirrhosis, primary biliary; Crohn's disease; | |
| | 2, p40) | | | | | Infection, hepatitis-B virus; Inflammatory | |
| | | | | | | bowel disease, general; Multiple sclerosis, | |
| | | | | | | general; Palmoplantar pustulosis; Poisoning, | |
| | | | | | | radiation; Psoriasis; Sarcoidosis | |
| IL12RB2 | interleukin 12 | Primary | 26 | rs17129789-C | Primary biliary | Arthritis, rheumatoid; Cancer, lymphoma, T- | HIV DNA vaccine, Dong-A/Genexine; IL-12, OncoSec |
| | receptor, beta 2 | biliary | | (0.18), | cirrhosis | cell; Cancer, melanoma; Cancer, | Medical; IL-2 + IL-12, Roche; Apilimod mesylate; |
| | | cirrhosis | | rs3790567-A | | neuroendocrine, Merkel cell carcinoma; | Interleukin-12; Wye |
| | | | | (0.232), | | Cancer, renal; Cancer, sarcoma, Kaposi's; | |
| | | | | rs3790567-A | | Crohn's disease; Immunodeficiency, IgG | |
| | | | | (0.24) | | deficiency; Infection, HIV/AIDS; Infection, | |
| | | | | | | hepatitis-C virus; Multiple sclerosis, general; | |
| | | | | | | Psoriasis | |
| IL12RB2 | interleukin 12 | Behcet's | 3 | rs1495965-G | Behcet's disease | Arthritis, rheumatoid; Cancer, lymphoma, T- | HIV DNA vaccine, Dong-A/Genexine; IL-12, OncoSec |
| | receptor, beta 2 | disease | | (0.51), | | cell; Cancer, melanoma; Cancer, | Medical; IL-2 + IL-12, Roche; Apilimod mesylate; |
| | | | | rs924080-? | | neuroendocrine, Merkel cell carcinoma; | Interleukin-12, Wye |
| | | | | (NR) | | Cancer, renal; Cancer, sarcoma, Kaposi's; | |
| | | | | | | Crohn's disease; Immunodeficiency, IgG | |
| | | | | | | deficiency; Infection, HIV/AIDS; Infection, | |
| | | | | | | hepatitis-C virus; Multiple sclerosis, general; | |
| | | | | | | Psoriasis | |
| IL13 | interleukin 13 | Psoriasis | 30 | rs20541-G | Psoriasis | Addiction, nicotine; Asthma; Cancer, brain; | 56076; IL-13 siRNAs, Allerna Therapeutics; QAX-576; |
| | | | | (0.79) | | Cancer, lymphoma, Hodgkin's; Chronic | SAR-156597; S8-313; Anrukinzumab; Lebrikizumab; |
| | | | | | | obstructive pulmonary disease; Colitis, | Nadolol, Inverseon; Tralokinum |
| | | | | | | ulcerative; Crohn's disease; Fibrosis, | |
| | | | | | | pulmonary, idiopathic; Rhinitis, allergic, | |
| | | | | | | seasonal; Wound healing | |
| IL15 | interleukin 15 | Response to | 8 | rs17007695-C | Response to | Arthritis, rheumatoid; Inflammation, general; | AMG-714; DISC-02 |
| | | treatment for | | (NR) | treatment for | Psoriasis | |
| | | acute | | | acute | | |
| | | lymphoblastic | | | lymphoblastic | | |
| | | leukemia | | | leukemia | | |
| IL18R1 | interleukin 18 | Celiac disease | 65 | rs13015714-C | Celiac disease | Cancer, lymphoma, B-cell; Cancer, lymphoma, | VX-765; Iboctadekin; Iboctadekin + doxorubicin; |
| | receptor 1 | | | (NR), | | Hodgkin's; Cancer, lymphoma, non-Hodgkin's; | Iboctadekin + rituxim |
| | | | | rs917997-A | | Cancer, melanoma; Cancer, renal; Epilepsy, | |
| | | | | (0.24) | | partial (focal, local); Inflammation, general; | |
| | | | | | | Psoriasis | |
| IL18R1 | interleukin 18 | Asthma | 19 | rs3771166-G | Asthma | Cancer, lymphoma, B-cell; Cancer, lymphoma, | VX-765; Iboctadekin; Iboctadekin + doxorubicin; |
| | receptor 1 | | | (0.62) | | Hodgkin's; Cancer, lymphoma, non-Hodgkin's; | Iboctadekin + rituxim |
| | | | | | | Cancer, melanoma; Cancer, renal; Epilepsy, | |
| | | | | | | partial (focal, local); Inflammation, general; | |
| | | | | | | Psoriasis | |
| IL1A | interleukin 1, | Endometriosis | 4 | rs6542095-C | Endometriosis | Arthritis, rheumatoid; Atherosclerosis; Cancer, | MABp1; Arthritis therapy, NanoSmart |
| | alpha | | | (0.72) | | general; Cancer, leukaemia, acute lymphocytic; | |
| | | | | | | Cancer, leukaemia, acute myelogenous; | |
| | | | | | | Cancer, leukaemia, chronic lymphocytic; | |
| | | | | | | Cancer, leukaemia, chronic myelogenous; | |
| | | | | | | Cancer, leukaemia, chronic myelomonocytic; | |
| | | | | | | Cancer, liver; Cancer, lymphoma, general; | |
| | | | | | | Cancer, solid, general; Diabetes, Type 2; | |
| | | | | | | Idiopathic myelofibrosis; Myelodysplastic | |
| | | | | | | syndrome; Reperfusion injury; Restenosis; | |
| | | | | | | Thrombosis, general | |
| IL1R2 | interleukin 1 | Ulcerative | 96 | rs2310173-T | Ulcerative colitis | Anaemia, renal disease-induced; Arthritis, | 9683; AMG-108; ARRY-438162; CDP-484; PMI-005; |
| | receptor, type II | colitis | | (0.46) | | juvenile; Arthritis, osteo; Arthritis, rheumatoid; | Givinostat; Rilonace |
| | | | | | | Cancer, biliary; Cancer, breast; Cancer, | |
| | | | | | | colorectal; Cancer, leukaemia, acute | |
| | | | | | | myelogenous; Cancer, lung, non-small cell; | |
| | | | | | | Cancer, lymphoma, Hodgkin's; Cancer, | |
| | | | | | | melanoma; Cancer, myeloma; Cancer, | |
| | | | | | | pancreatic; Cancer, solid, general; Chronic | |
| | | | | | | obstructive pulmonary disease; Diabetes, Type | |
| | | | | | | 2; Familial cold autoinflammatory syndrome; | |
| | | | | | | Hyperuricaemia; Idiopathic myelofibrosis; | |
| | | | | | | Inflammation, general; Irritable bowel | |
| | | | | | | syndrome; Muckle-Wells syndrome; Mucositis, | |
| | | | | | | general; Polycythaemia vera; | |
| | | | | | | Thrombocythaemia; Thrombophlebitis | |
| IL2 | interleukin 2 | Type 1 | 50 | rs4505848-? | Type 1 diabetes | Cancer, brain; Cancer, breast; Cancer, cervical; | IL-2 Cancer Gene Medicine; IL-2 gene therapy, |
| | | diabetes | | (NR) | | Cancer, colorectal; Cancer, head and neck; | Targeted Genetics; NTC-121; Reximmune-C; TG-4010; |
| | | | | | | Cancer, liver; Cancer, lung, non-small cell; | Tipapkinogene sovaciv |
| | | | | | | Cancer, ovarian; Cancer, pancreatic; Cancer, | |
| | | | | | | prostate; Cancer, renal; Cancer, sarcoma, soft | |
| | | | | | | tissue; Dysplasia, cervical; Infection, HIV/AIDS; | |
| | | | | | | Infection, cytomegalovirus; Infection, human | |
| | | | | | | papilloma virus | |
| IL2 | interleukin 2 | Celiac disease | 65 | rs13151961-? | Celiac disease | Cancer, brain; Cancer, breast; Cancer, cervical; | IL-2 Cancer Gene Medicine; IL-2 gene therapy, |
| | | | | (0.86), | | Cancer, colorectal; Cancer, head and neck; | Targeted Genetics; NTC-121; Reximmune-C; TG-4010; |
| | | | | rs6822844-C | | Cancer, liver; Cancer, lung, non-small cell; | Tipapkinogene sovaciv |
| | | | | (0.81), | | Cancer, ovarian; Cancer, pancreatic; Cancer, | |
| | | | | rs6822844-G | | prostate; Cancer, renal; Cancer, sarcoma, soft | |
| | | | | (0.82) | | tissue; Dysplasia, cervical; Infection, HIV/AIDS; | |
| | | | | | | Infection, cytomegalovirus; Infection, human | |
| | | | | | | papilloma virus | |
| IL2 | interleukin 2 | Rheumatoid | 49 | rs13119723-A | Rheumatoid | Cancer, brain; Cancer, breast; Cancer, cervical; | IL-2 Cancer Gene Medicine; IL-2 gene therapy, |
| | | arthritis | | (0.15) | arthritis | Cancer, colorectal; Cancer, head and neck; | Targeted Genetics; NTC-121; Reximmune-C; TG-4010; |
| | | | | | | Cancer, liver; Cancer, lung, non-small cell; | Tipapkinogene sovaciv |
| | | | | | | Cancer, ovarian; Cancer, pancreatic; Cancer, | |
| | | | | | | prostate; Cancer, renal; Cancer, sarcoma, soft | |
| | | | | | | tissue; Dysplasia, cervical; Infection, HIV/AIDS; | |
| | | | | | | Infection, cytomegalovirus; Infection, human | |
| | | | | | | papilloma virus | |
| IL2 | interleukin 2 | Alopecia | 11 | rs7682241-A | Alopecia areata | Cancer, brain; Cancer, breast; Cancer, cervical; | IL-2 Cancer Gene Medicine; IL-2 gene therapy, |
| | | areata | | (0.33) | | Cancer, colorectal; Cancer, head and neck; | Targeted Genetics; NTC-121; Reximmune-C; TG-4010; |
| | | | | | | Cancer, liver; Cancer, lung, non-small cell; | Tipapkinogene sovaciv |
| | | | | | | Cancer, ovarian; Cancer, pancreatic; Cancer, | |
| | | | | | | prostate; Cancer, renal; Cancer, sarcoma, soft | |
| | | | | | | tissue; Dysplasia, cervical; Infection, HIV/AIDS; | |
| | | | | | | Infection, cytomegalovirus; Infection, human | |
| | | | | | | papilloma virus | |
| IL2 | interleukin 2 | Ulcerative | 96 | rs17388568-A | Ulcerative colitis | Cancer, brain; Cancer, breast; Cancer, cervical; | IL-2 Cancer Gene Medicine; IL-2 gene therapy, |
| | | colitis | | (0.27) | | Cancer, colorectal; Cancer, head and neck; | Targeted Genetics; NTC-121; Reximmune-C; TG-4010; |
| | | | | | | Cancer, liver; Cancer, lung, non-small cell; | Tipapkinogene sovaciv |
| | | | | | | Cancer, ovarian; Cancer, pancreatic; Cancer, | |
| | | | | | | prostate; Cancer, renal; Cancer, sarcoma, soft | |
| | | | | | | tissue; Dysplasia, cervical; Infection, HIV/AIDS; | |
| | | | | | | Infection, cytomegalovirus; Infection, human | |
| | | | | | | papilloma virus | |
| IL21R | interleukin 21 | Bone mineral | 42 | rs8057551-G | Bone mineral | Arthritis, rheumatoid; Cancer, colorectal; | ATR-107; NNC-114-0005; Denenicok |
| | receptor | density | | (0.32) | density | Cancer, lymphoma, B-cell; Cancer, lymphoma, | |
| | | | | | | non-Hodgkin's; Cancer, melanoma; Cancer, | |
| | | | | | | ovarian; Cancer, renal; Crohn's disease; Lupus | |
| | | | | | | erythematosus, systemic | |
| IL23R | interleukin 23 | Ankylosing | 7 | rs11209026-? | Ankylosing | Arthritis, rheumatoid; Inflammatory bowel | ATX-3105; FM-2 |
| | receptor | spondylitis | | (0.94) | spondylitis | disease, general; Multiple sclerosis, general; | |
| | | | | | | Psoriasis | |
| IL23R | interleukin 23 | Crohn's | 83 | 17 marker | Crohn's disease | Arthritis, rheumatoid; Inflammatory bowel | ATX-3105; FM-2 |
| | receptor | disease | | haplotype-1 | | disease, general; Multiple sclerosis, general; | |
| | | | | (0.23), 17 | | Psoriasis | |
| | | | | marker | | | |
| | | | | haplotype-2 | | | |
| | | | | (0.97), | | | |
| | | | | rs11209026-? | | | |
| | | | | (0.92), | | | |
| | | | | rs11209026-G | | | |
| | | | | (0.93), | | | |
| | | | | rs11465804-? | | | |
| | | | | (NR), | | | |
| | | | | rs11465804-T | | | |
| | | | | (0.93), | | | |
| | | | | rs11805303-T | | | |
| | | | | (0.32), | | | |
| | | | | rs7517847-? | | | |
| | | | | (0.40) | | | |
| IL23R | interleukin 23 | Inflammatory | 17 | rs11209026-? | Inflammatory | Arthritis, rheumatoid; Inflammatory bowel | ATX-3105; FM-2 |
| | receptor | bowel disease | | (0.94), | bowel disease | disease, general; Multiple sclerosis, general; | |
| | | | | rs11209026-G | | Psoriasis | |
| | | | | (0.93), | | | |
| | | | | rs7517847-C | | | |
| | | | | (0.56) | | | |
| IL23R | interleukin 23 | Behcet's | 3 | rs1495965-G | Behcet's disease | Arthritis, rheumatoid; Inflammatory bowel | ATX-3105; FM-2 |
| | receptor | disease | | (0.51), | | disease, general; Multiple sclerosis, general; | |
| | | | | rs924080-? | | Psoriasis | |
| | | | | (NR) | | | |
| IL27 | interleukin 27 | Type 1 | 50 | rs4788084-G | Type 1 diabetes | Adrenoleukodystrophy; Arthritis, rheumatoid; | RPI-7 |
| | | diabetes | | (0.42) | | Infection, HIV/AIDS; Infection, herpes virus, | |
| | | | | | | unspecified; Infection, rabies; Infection, | |
| | | | | | | varicella zoster virus; Multiple sclerosis, | |
| | | | | | | general; Myasthenia gravis | |
| IL27 | interleukin 27 | Crohn's | 83 | rs151181-G | Crohn's disease | Adrenoleukodystrophy; Arthritis, rheumatoid; | RPI-7 |
| | | disease | | (0.39) | | Infection, HIV/AIDS; Infection, herpes virus, | |
| | | | | | | unspecified; Infection, rabies; Infection, | |
| | | | | | | varicella zoster virus; Multiple sclerosis, | |
| | | | | | | general; Myasthenia gravis | |
| IL27 | interleukin 27 | Inflammatory | 17 | rs8049439-G | Inflammatory | Adrenoleukodystrophy; Arthritis, rheumatoid; | RPI-7 |
| | | bowel disease | | (0.37) | bowel disease | Infection, HIV/AIDS; Infection, herpes virus, | |
| | | | | | | unspecified; Infection, rabies; Infection, | |
| | | | | | | varicella zoster virus; Multiple sclerosis, | |
| | | | | | | general; Myasthenia gravis | |
| IL28RA | interleukin 28 | Psoriasis | 30 | rs4649203-A | Psoriasis | Infection, hepatitis-B virus; Infection, hepatitis- | Interleukin-29, ZymoGeneti |
| | receptor, alpha | | | (0.73) | | C virus | |
| | (interferon, | | | | | | |
| | lambda receptor) | | | | | | |
| IL2RA | interleukin 2 | Crohn's | 83 | rs12722489-C | Crohn's disease | Arthritis, rheumatoid; Asthma; Burns; Cancer, | ALT-801; CYT-91000; EMD-273063; IL-2 + IL-12, Roche; |
| | receptor, alpha | disease | | (0.85) | | bladder; Cancer, brain; Cancer, breast; Cancer, | MT-204; NHS-IL2-LT; PRO-1556; WuTac; Aldesleukin; |
| | | | | | | cervical; Cancer, colorectal; Cancer, head and | Aldesleukin, Zenotech; Anti-IL2 receptor MAb, |
| | | | | | | neck; Cancer, leukaemia, acute myelogenous; | Seragen; Basiliximab; Celmoleukin; Daclizumab; |
| | | | | | | Cancer, leukaemia, chronic lymphocytic; | Darleukin; Denileukin diftitox; Interleukin-2 inhibitors, |
| | | | | | | Cancer, leukaemia, general; Cancer, liver; | Sune; Interleukin-2, 2nd-gen, 3SBio; Interleukin-2, |
| | | | | | | Cancer, lung, non-small cell; Cancer, lung, small | Ajinomoto; Interleukin-2, Biotech; Interleukin-2, |
| | | | | | | cell; Cancer, lymphoma, Hodgkin's; Cancer, | Medusa, Flamel; Interleukin-2, Novartis; Interleukins, |
| | | | | | | lymphoma, T-cell; Cancer, lymphoma, general; | Cel-Sci; Teceleukin; Thymotrinan; Tucotuzumab |
| | | | | | | Cancer, lymphoma, non-Hodgkin's; Cancer, | celmoleuk |
| | | | | | | melanoma; Cancer, nasopharyngeal; Cancer, | |
| | | | | | | neuroendocrine, neuroblastoma; Cancer, | |
| | | | | | | ovarian; Cancer, pancreatic; Cancer, prostate; | |
| | | | | | | Cancer, renal; Cancer, sarcoma, general; | |
| | | | | | | Cancer, solid, general; Cancer, squamous cell; | |
| | | | | | | Cancer, urethral; Colitis, ulcerative; Diabetes, | |
| | | | | | | Type 1; Dysplasia, cervical; Eczema, atopic; | |
| | | | | | | Endometriosis; Head trauma; Infection, | |
| | | | | | | Brucella; Infection, Chlamydia; Infection, | |
| | | | | | | HIV/AIDS; Infection, bone; Infection, | |
| | | | | | | cytomegalovirus; Infection, dermatological; | |
| | | | | | | Infection, gynaecological; Infection, | |
| | | | | | | haemorrhagic fever; Infection, hepatitis-C | |
| | | | | | | virus; Infection, herpes simplex virus; Infection, | |
| | | | | | | intra-abdominal, unspecified; Infection, | |
| | | | | | | meningitis, viral; Infection, pneumonia, | |
| | | | | | | hospital-acquired; Infection, tuberculosis; | |
| | | | | | | Infection, urinary tract, unspecified; Infection, | |
| | | | | | | varicella zoster virus; Inflammation, prostate; | |
| | | | | | | Lupus erythematosus, systemic; Multiple | |
| | | | | | | sclerosis, general; Multiple sclerosis, | |
| | | | | | | progressive, secondary; Multiple sclerosis, | |
| | | | | | | relapsing-remitting; Nephritis, general; | |
| | | | | | | Pancreatitis; Psoriasis; Sepsis; Sinusitis; | |
| | | | | | | Transplant rejection, bone marrow; Transplant | |
| | | | | | | rejection, general; Ulcer, diabetic; Ulcer, | |
| | | | | | | gastric; Uveitis; Wound healing | |
| IL2RA | interleukin 2 | Alopecia | 11 | rs3118470-G | Alopecia areata | Arthritis, rheumatoid; Asthma; Burns; Cancer, | ALT-801; CYT-91000; EMD-273063; IL-2 + IL-12, Roche; |
| | receptor, alpha | areata | | (0.30) | | bladder; Cancer, brain; Cancer, breast; Cancer, | MT-204; NHS-IL2-LT; PRO-1556; WuTac; Aldesleukin; |
| | | | | | | cervical; Cancer, colorectal; Cancer, head and | Aldesleukin, Zenotech; Anti-IL2 receptor MAb, |
| | | | | | | neck; Cancer, leukaemia, acute myelogenous; | Seragen; Basiliximab; Celmoleukin; Daclizumab; |
| | | | | | | Cancer, leukaemia, chronic lymphocytic; | Darleukin; Denileukin diftitox; Interleukin-2 inhibitors, |
| | | | | | | Cancer, leukaemia, general; Cancer, liver; | Sune; Interleukin-2, 2nd-gen, 3SBio; Interleukin-2, |
| | | | | | | Cancer, lung, non-small cell; Cancer, lung, small | Ajinomoto; Interleukin-2, Biotech; Interleukin-2, |
| | | | | | | cell; Cancer, lymphoma, Hodgkin's; Cancer, | Medusa, Flamel; Interleukin-2, Novartis; Interleukins, |
| | | | | | | lymphoma, T-cell; Cancer, lymphoma, general; | Cel-Sci; Teceleukin; Thymotrinan; Tucotuzumab |
| | | | | | | Cancer, lymphoma, non-Hodgkin's; Cancer, | celmoleuk |
| | | | | | | melanoma; Cancer, nasopharyngeal; Cancer, | |
| | | | | | | neuroendocrine, neuroblastoma; Cancer, | |
| | | | | | | ovarian; Cancer, pancreatic; Cancer, prostate; | |
| | | | | | | Cancer, renal; Cancer, sarcoma, general; | |
| | | | | | | Cancer, solid, general; Cancer, squamous cell; | |
| | | | | | | Cancer, urethral; Colitis, ulcerative; Diabetes, | |
| | | | | | | Type 1; Dysplasia, cervical; Eczema, atopic; | |
| | | | | | | Endometriosis; Head trauma; Infection, | |
| | | | | | | Brucella; Infection, Chlamydia; Infection, | |
| | | | | | | HIV/AIDS; Infection, bone; Infection, | |
| | | | | | | cytomegalovirus; Infection, dermatological; | |
| | | | | | | Infection, gynaecological; Infection, | |
| | | | | | | haemorrhagic fever; Infection, hepatitis-C | |
| | | | | | | virus; Infection, herpes simplex virus; Infection, | |
| | | | | | | intra-abdominal, unspecified; Infection, | |
| | | | | | | meningitis, viral; Infection, pneumonia, | |
| | | | | | | hospital-acquired; Infection, tuberculosis; | |
| | | | | | | Infection, urinary tract, unspecified; Infection, | |
| | | | | | | varicella zoster virus; Inflammation, prostate; | |
| | | | | | | Lupus erythematosus, systemic; Multiple | |
| | | | | | | sclerosis, general; Multiple sclerosis, | |
| | | | | | | progressive, secondary; Multiple sclerosis, | |
| | | | | | | relapsing-remitting; Nephritis, general; | |
| | | | | | | Pancreatitis; Psoriasis; Sepsis; Sinusitis; | |
| | | | | | | Transplant rejection, bone marrow; Transplant | |
| | | | | | | rejection, general; Ulcer, diabetic; Ulcer, | |
| | | | | | | gastric; Uveitis; Wound healing | |
| IL2RA | interleukin 2 | Vitiligo | 25 | rs706779-A | Vitiligo | Arthritis, rheumatoid; Asthma; Burns; Cancer, | ALT-801; CYT-91000; EMD-273063; IL-2 + IL-12, Roche; |
| | receptor, alpha | | | (0.535) | | bladder; Cancer, brain; Cancer, breast; Cancer, | MT-204; NHS-IL2-LT; PRO-1556; WuTac; Aldesleukin; |
| | | | | | | cervical; Cancer, colorectal; Cancer, head and | Aldesleukin, Zenotech; Anti-IL2 receptor MAb, |
| | | | | | | neck; Cancer, leukaemia, acute myelogenous; | Seragen; Basiliximab; Celmoleukin; Daclizumab; |
| | | | | | | Cancer, leukaemia, chronic lymphocytic; | Darleukin; Denileukin diftitox; Interleukin-2 inhibitors, |
| | | | | | | Cancer, leukaemia, general; Cancer, liver; | Sune; Interleukin-2, 2nd-gen, 3SBio; Interleukin-2, |
| | | | | | | Cancer, lung, non-small cell; Cancer, lung, small | Ajinomoto; Interleukin-2, Biotech; Interleukin-2, |
| | | | | | | cell; Cancer, lymphoma, Hodgkin's; Cancer, | Medusa, Flamel; Interleukin-2, Novartis; Interleukins, |
| | | | | | | lymphoma, T-cell; Cancer, lymphoma, general; | Cel-Sci; Teceleukin; Thymotrinan; Tucotuzumab |
| | | | | | | Cancer, lymphoma, non-Hodgkin's; Cancer, | celmoleuk |
| | | | | | | melanoma; Cancer, nasopharyngeal; Cancer, | |
| | | | | | | neuroendocrine, neuroblastoma; Cancer, | |
| | | | | | | ovarian; Cancer, pancreatic; Cancer, prostate; | |
| | | | | | | Cancer, renal; Cancer, sarcoma, general; | |
| | | | | | | Cancer, solid, general; Cancer, squamous cell; | |
| | | | | | | Cancer, urethral; Colitis, ulcerative; Diabetes, | |
| | | | | | | Type 1; Dysplasia, cervical; Eczema, atopic; | |
| | | | | | | Endometriosis; Head trauma; Infection, | |
| | | | | | | Brucella; Infection, Chlamydia; Infection, | |
| | | | | | | HIV/AIDS; Infection, bone; Infection, | |
| | | | | | | cytomegalovirus; Infection, dermatological; | |
| | | | | | | Infection, gynaecological; Infection, | |
| | | | | | | haemorrhagic fever; Infection, hepatitis-C | |
| | | | | | | virus; Infection, herpes simplex virus; Infection, | |
| | | | | | | intra-abdominal, unspecified; Infection, | |
| | | | | | | meningitis, viral; Infection, pneumonia, | |
| | | | | | | hospital-acquired; Infection, tuberculosis; | |
| | | | | | | Infection, urinary tract, unspecified; Infection, | |
| | | | | | | varicella zoster virus; Inflammation, prostate; | |
| | | | | | | Lupus erythematosus, systemic; Multiple | |
| | | | | | | sclerosis, general; Multiple sclerosis, | |
| | | | | | | progressive, secondary; Multiple sclerosis, | |
| | | | | | | relapsing-remitting; Nephritis, general; | |
| | | | | | | Pancreatitis; Psoriasis; Sepsis; Sinusitis; | |
| | | | | | | Transplant rejection, bone marrow; Transplant | |
| | | | | | | rejection, general; Ulcer, diabetic; Ulcer, | |
| | | | | | | gastric; Uveitis; Wound healing | |
| IL2RB | interleukin 2 | Asthma | 19 | rs2284033-G | Asthma | Cancer, leukaemia, general; Cancer, lung, non- | CYT-91000; NHS-IL2- |
| | receptor, beta | | | (0.56) | | small cell; Cancer, lymphoma, T-cell; Cancer, | |
| | | | | | | lymphoma, non-Hodgkin's; Cancer, solid, | |
| | | | | | | general | |
| IL2RB | interleukin 2 | Rheumatoid | 49 | rs743777-G | Rheumatoid | Cancer, leukaemia, general; Cancer, lung, non- | CYT-91000; NHS-IL2- |
| | receptor, beta | arthritis | | (NR) | arthritis | small cell; Cancer, lymphoma, T-cell; Cancer, | |
| | | | | | | lymphoma, non-Hodgkin's; Cancer, solid, | |
| | | | | | | general | |
| IL3 | interleukin 3 | Crohn's | 83 | rs12521868-T | Crohn's disease | Cancer, prostate; Cancer, sarcoma, general | IL-3 gene therapy, Crucell; Prostate cancer ther, V |
| | (colony- | disease | | (0.42) | | | |
| | stimulating | | | | | | |
| | factor, multiple) | | | | | | |
| IL3RA | interleukin 3 | Schizophrenia | 33 | rs4129148-C | Schizophrenia | Anaemia, aplastic; Anaemia, | CSL-360; CSL-362; PEG-interleukin-3, Wyeth; SL-401; |
| | receptor, alpha | | | (NR) | | radio/chemotherapy-induced; Arthritis, | SL-501; Anti-IL3 MAb, SuppreMol; Daniplestim; |
| | (low affinity) | | | | | rheumatoid; Cancer, breast; Cancer, general; | Leridistim; Muplestim, Cangene; Muplestim, Wye |
| | | | | | | Cancer, haematological, general; Cancer, | |
| | | | | | | leukaemia, acute myelogenous; Cancer, | |
| | | | | | | leukaemia, chronic myelogenous; Cancer, | |
| | | | | | | leukaemia, general; Immunodeficiency, | |
| | | | | | | general; Myelodysplastic syndrome; | |
| | | | | | | Neutropenia, general; Radio/chemotherapy- | |
| | | | | | | induced injury, bone marrow, | |
| | | | | | | thrombocytopenia; Radio/chemotherapy- | |
| | | | | | | induced injury, general | |
| IL6 | interleukin 6 | Dialysis- | 31 | rs17364464-? | Dialysis-related | Arthritis, general; Arthritis, rheumatoid; | ALD-518; FM-101; OPR-003; Anti-IL-6 MAb, |
| | (interferon, beta | related | | (0.08) | mortality | Cachexia; Cancer fatigue; Cancer, colorectal; | MedImmune; Olokizumab (IV); Siltuximab; Sirukum |
| | 2) | mortality | | | | Cancer, haematological, general; Cancer, head | |
| | | | | | | and neck; Cancer, lung, non-small cell; Cancer, | |
| | | | | | | lymphoma, non-Hodgkin's; Cancer, myeloma; | |
| | | | | | | Cancer, ovarian; Cancer, pancreatic; Cancer, | |
| | | | | | | prostate; Cancer, renal; Cancer, solid, general; | |
| | | | | | | Castleman's disease; Crohn's disease; | |
| | | | | | | Inflammation, general; Lupus erythematosus, | |
| | | | | | | cutaneous; Lupus erythematosus, systemic; | |
| | | | | | | Lupus nephritis; Waldenstrom's | |
| | | | | | | hypergammaglobulinaemia | |
| IL6R | interleukin 6 | Pulmonary | 8 | rs4129267-? | Pulmonary | Ankylosing spondylitis; Arthritis, juvenile; | ALX-0061; AM-87; ARRY-438162; IL-6 antagonists, |
| | receptor | function traits | | (NR) | function traits | Arthritis, rheumatoid; Asthma; Cancer, biliary; | Protagonist Therapeutics; Pharmaprojects No. 6000; |
| | | | | | | Cancer, breast; Cancer, colorectal; Cancer, | SA-237; Interferon alpha + oncostatin M, Hepacyl |
| | | | | | | lung, non-small cell; Cancer, melanoma; | Therapeutics; Interleukin-6 fusion toxin, Lig; |
| | | | | | | Cancer, myeloma; Cancer, pancreatic; Cancer, | Interleukin-6 receptor MAb, Ch; Interleukin-6, |
| | | | | | | sarcoma, Kaposi's; Cancer, solid, general; | Ajinomoto; Interleukin-6, Cangene; Interleukin-6, |
| | | | | | | Castleman's disease; Chronic obstructive | Wyeth; Medroxyprogesterone, Inkine; Sarilumab; |
| | | | | | | pulmonary disease; Colitis, ulcerative; | Tocilizumab; Tocilizumab, BioXpress; Vesnarino |
| | | | | | | Conjunctivitis, inflammatory; Crohn's disease; | |
| | | | | | | Heart failure; Infection, HIV/AIDS; | |
| | | | | | | Inflammation, general; Inflammatory bowel | |
| | | | | | | disease, general; Irritable bowel syndrome; | |
| | | | | | | Lupus erythematosus, systemic; Psoriasis; | |
| | | | | | | Radio/chemotherapy-induced injury, bone | |
| | | | | | | marrow, neutropenia; Radio/chemotherapy- | |
| | | | | | | induced injury, bone marrow, | |
| | | | | | | thrombocytopenia; Rhinitis, allergic, general; | |
| | | | | | | Thrombocytopenia, general; | |
| | | | | | | Thrombocytopenic purpura; Urticaria | |
| IL7 | interleukin 7 | Longevity | 23 | rs2717536-A | Longevity | Cancer, breast; Cancer, colorectal; Cancer, | MGN-16 |
| | | | | (0.14) | | lung, general; Cancer, melanoma; Cancer, renal | |
| IL7R | interleukin 7 | Type 1 | 50 | rs6897932-G | Type 1 diabetes | Cancer, general; Immunodeficiency, general | IL-7-Dap 389 fusion toxin; Interleukin-7, Cytheris; |
| | receptor | diabetes | | (0.71) | | | Interleukin-7, IC Innovatio |
| IL7R | interleukin 7 | Ulcerative | 96 | rs3194051-G | Ulcerative colitis | Cancer, general; Immunodeficiency, general | IL-7-Dap 389 fusion toxin; Interleukin-7, Cytheris; |
| | receptor | colitis | | (0.27) | | | Interleukin-7, IC Innovatio |
| IL7R | interleukin 7 | Primary | 26 | rs860413-A | Primary biliary | Cancer, general; Immunodeficiency, general | IL-7-Dap 389 fusion toxin; Interleukin-7, Cytheris; |
| | receptor | biliary | | (0.72) | cirrhosis | | Interleukin-7, IC Innovatio |
| | | cirrhosis | | | | | |
| IL7R | interleukin 7 | Multiple | 59 | rs6897932-C | Multiple | Cancer, general; Immunodeficiency, general | IL-7-Dap 389 fusion toxin; Interleukin-7, Cytheris; |
| | receptor | sclerosis | | (0.75), | sclerosis | | Interleukin-7, IC Innovatio |
| | | | | rs931555-? | | | |
| | | | | (NR) | | | |
| INS | insulin | Prostate | 30 | rs7127900-A | Prostate cancer | Diabetes, Type 1; Diabetes, Type 2 | EG-02; Encellin XP; NNC-0123-0000-0338; Insulin, |
| | | cancer | | (0.20) | | | targeted, Merck & Co; Islet cells, TheraCyte; |
| | | | | | | | Proinsulin, BayHill Therapeuti |
| INSR | insulin receptor | Diabetic | 34 | rs2115386-C | Diabetic | Acute coronary syndrome; Cardiomyopathy, | 14920; AGT-181; CJC-1525; EML-16257; HinsBet; |
| | | retinopathy | | (0.55) | retinopathy | diabetic; Diabetes, Type 1; Diabetes, Type 2; | Insulin Aspart; Insulin Aspart, biphasic-2, No; Insulin |
| | | | | | | Diabetes, general; Diabetes, gestational; | Aspart, biphasic-3, Novo Nordisk; KRX-6 13; NN-1218; |
| | | | | | | Impaired glucose tolerance; Infarction, | NN-1952; NNC 0100-0454/NNC 90-1170; P30; PRO- |
| | | | | | | myocardial; Insulin-related metabolic | 001, ProRetina; SIA-II Insulin, Extended Delivery; |
| | | | | | | syndrome; Mucopolysaccharidosis I; Obesity; | XMetA; XMetS; Antidiabetic, Fate Therapeutics; Insulin + |
| | | | | | | Retinitis pigmentosa; Retinopathy, diabetic | GLP-1, Ascendis Pharma; Insulin Aspart, biphasic, |
| | | | | | | | Novo Nordisk; Insulin analogue, Lilly; Insulin aspart, |
| | | | | | | | Biocon; Insulin degludec; Insulin degludec + insulin |
| | | | | | | | aspart; Insulin detemir; Insulin glargine; Insulin |
| | | | | | | | glargine + lixisenatide; Insulin glargine, Biocon; Insulin |
| | | | | | | | glargine, Biodel; Insulin glargine, Gan & Lee |
| | | | | | | | Pharmaceutical; Insulin glargine, Lilly; Insulin glargine, |
| | | | | | | | Wockhardt; Insulin glulisine; Insulin lispro, Biocon; |
| | | | | | | | Insulin mimetic, Biota; Insulin, AERx; Insulin, Access; |
| | | | | | | | Insulin, Advancell; Insulin, Alkermes, inhaled; Insulin, |
| | | | | | | | Apricus Biosciences; Insulin, BEODAS; Insulin, Chiron; |
| | | | | | | | Insulin, Cpex, intranasal; Insulin, Ferring Pharma; |
| | | | | | | | Insulin, Genentech, recombinant; Insulin, Medusa, |
| | | | | | | | Flamel-2; Insulin, Novo Nordisk; Insulin, Orin; Insulin, |
| | | | | | | | PharmFilm; Insulin, Phosphagenics; Insulin, PolyXen, |
| | | | | | | | Lipoxen; Insulin, ProStrakan; Insulin, Sanofi Aventis; |
| | | | | | | | Insulin, SemBioSys; Insulin, Spiros; Insulin, |
| | | | | | | | Technosphere, Mannkind; Insulin, Wockhardt; Insulin, |
| | | | | | | | Zymo, recombinant; Insulin, basal, Lilly; Insulin, basal, |
| | | | | | | | adjustable, Biodel; Insulin, buccal, Generex; Insulin, |
| | | | | | | | hydrogel, Ascendis; Insulin, inhaled, Dance Pharma; |
| | | | | | | | Insulin, inhaled, Nektar; Insulin, monocomponent, |
| | | | | | | | Novo; Insulin, nasal, DelSite; Insulin, nasal, Novo |
| | | | | | | | Nordisk; Insulin, oral, Apollo; Insulin, oral, Biocon-2; |
| | | | | | | | Insulin, oral, Biolaxy; Insulin, oral, CM&D Pharma; |
| | | | | | | | Insulin, oral, Emisphere-2; Insulin, oral, Oramed; |
| | | | | | | | Insulin, oral, Oshadi Drug Administration; Insulin, |
| | | | | | | | rapid-acting analogue + PH20, Halozyme; Insulin, |
| | | | | | | | rapid-acting analogue, Biodel; Insulin, recombinant, |
| | | | | | | | Aventis; Insulin, regular human, Biodel; Insulin, short- |
| | | | | | | | acting regular + PH20, Halozyme; Insulin, sublingual, |
| | | | | | | | Biodel; Oral insulin, C |
| IRS1 | insulin receptor | Type 2 | 61 | rs7578326-A | Type 2 diabetes | Corneal injury; Glaucoma; Macular | Aganirsen (intravitrea |
| | substrate 1 | diabetes | | (NR) | | degeneration, age-related, general; Psoriasis; | |
| | | | | | | Retinopathy, diabetic; Retinopathy, general; | |
| | | | | | | Rosacea | |
| IRS1 | insulin receptor | Type 2 | 7 | rs2943641-C | Type 2 diabetes | Corneal injury; Glaucoma; Macular | Aganirsen (intravitrea |
| | substrate 1 | diabetes and | | (0.63) | and other traits | degeneration, age-related, general; Psoriasis; | |
| | | other traits | | | | Retinopathy, diabetic; Retinopathy, general; | |
| | | | | | | Rosacea | |
| ITGA11 | integrin, alpha 11 | Attention | 45 | rs7164335-? | Attention deficit | Arthritis, osteo; Arthritis, rheumatoid | Alpha11AY1 antagonist, Han |
| | | deficit | | (NR) | hyperactivity | | |
| | | hyperactivity | | | disorder | | |
| | | disorder | | | | | |
| ITGA4 | integrin, alpha 4 | Celiac disease | 65 | rs13010713-G | Celiac disease | Alopecia, androgenic; Arthritis, rheumatoid; | AS-101; BIO-1211; CDP-323; DW-908e; ELND-002; ISIS- |
| | (antigen CD49D, | | | (0.45) | | Asthma; Atherosclerosis; Cancer, | 107248; TBC-3342; TBC-772; TRK-170; VLA-4 |
| | alpha 4 subunit | | | | | haematological, general; Cancer, myeloma; | inhibitors, Elan; VLA-4 antagonists, Uriach; |
| | of VLA-4 | | | | | Colitis, ulcerative; Crohn's disease; Eczema, | Etrolizumab; Firategrast; Natalizumab; Natalizumab, |
| | receptor) | | | | | atopic; Infection, HIV/AIDS; Infection, human | BioXpress; Valategrast hydrochloride; Vedolizum |
| | | | | | | papilloma virus; Inflammatory bowel disease, | |
| | | | | | | general; Irritable bowel syndrome; Multiple | |
| | | | | | | sclerosis, general; Multiple sclerosis, | |
| | | | | | | progressive, secondary; Multiple sclerosis, | |
| | | | | | | relapsing-remitting; Psoriasis; | |
| | | | | | | Radio/chemotherapy-induced alopecia; | |
| | | | | | | Radio/chemotherapy-induced injury, bone | |
| | | | | | | marrow, general | |
| ITGA9 | integrin, alpha 9 | Nasopharyngeal | 9 | rs189897-A | Nasopharyngeal | Bone disorder, general; Cancer, general; | Anti-alpha9 integrin MAbs, G |
| | | carcinoma | | (0.09) | carcinoma | Immune disease, unspecified | |
| ITGAE | integrin, alpha E | Attention | 45 | rs220470-? | Attention deficit | Colitis, ulcerative | Etrolizum |
| | (antigen CD103, | deficit | | (NR) | hyperactivity | | |
| | human mucosal | hyperactivity | | | disorder | | |
| | lymphocyte | disorder | | | | | |
| | antigen 1; alpha | | | | | | |
| | polypeptide) | | | | | | |
| ITGAM | integrin, alpha M | Systemic | 42 | rs11150610-? | Systemic lupus | Asthma; Chronic obstructive pulmonary | SN-18915; Dual LFA-1/Mac-1 antagonist, Hoffmann-La |
| | (complement | lupus | | (0.42), | erythematosus | disease; Hepatic dysfunction, general | Roc |
| | component 3 | erythematosus | | rs11574637-C | | | |
| | receptor 3 | | | (0.19), | | | |
| | subunit) | | | rs9888739-T | | | |
| | | | | (0.13) | | | |
| ITGB1 | integrin, beta 1 | Depression-- | 9 | rs11009175-A | Depression-- | Arthritis, osteo; Arthritis, rheumatoid; Asthma; | 52363; BIO-1211; DI-17E6; DW-908e; Evatak; GBR- |
| | (fibronectin | quantitative | | (0.17) | quantitative | Atherosclerosis; Cancer, bone; Cancer, | 500; JSM-6427; PF-04605412; TBC-3342; TBC-772; |
| | receptor, beta | trait | | | trait | colorectal; Cancer, liver; Cancer, lung, non- | TRK-170; TRK-720; VLA-4 inhibitors, Elan; VLA-4 |
| | polypeptide, | | | | | small cell; Cancer, melanoma; Cancer, | antagonists, Uriach; Alpha11AY1 antagonist, Hansa; |
| | antigen CD29 | | | | | myeloma; Cancer, ovarian; Cancer, pancreatic; | Firategrast; Natalizumab; Natalizumab, BioXpress; |
| | includes MDF2, | | | | | Cancer, peritoneal; Cancer, prostate; Cancer, | Valategrast hydrochloride; Volocixim |
| | MSK12) | | | | | renal; Cancer, solid, general; Colitis, ulcerative; | |
| | | | | | | Crohn's disease; Inflammatory bowel disease, | |
| | | | | | | general; Irritable bowel syndrome; Macular | |
| | | | | | | degeneration, age-related, wet; Multiple | |
| | | | | | | sclerosis, general; Multiple sclerosis, relapsing- | |
| | | | | | | remitting | |
| ITGB6 | integrin, beta 6 | Nephropathy | 23 | rs4664308-? | Nephropathy | Cancer, bone; Cancer, colorectal; Cancer, | DI-17E6; STX-100; Integrin alphaVAY6 binding agent, |
| | | | | (0.43) | | endometrial; Cancer, liver; Cancer, lung, non- | CRT; Intetumum |
| | | | | | | small cell; Cancer, melanoma; Cancer, ovarian; | |
| | | | | | | Cancer, pancreatic; Cancer, peritoneal; Cancer, | |
| | | | | | | prostate; Cancer, renal; Cancer, sarcoma, | |
| | | | | | | general; Cancer, sarcoma, soft tissue; Cancer, | |
| | | | | | | solid, general; Diagnosis, cancer; Fibrosis, | |
| | | | | | | general; Fibrosis, pulmonary, idiopathic; | |
| | | | | | | Nephropathy, general | |
| ITGB6 | integrin, beta 6 | Type 2 | 61 | rs7593730-? | Type 2 diabetes | Cancer, bone; Cancer, colorectal; Cancer, | DI-17E6; STX-100; Integrin alphaVAY6 binding agent, |
| | | diabetes | | (0.78) | | endometrial; Cancer, liver; Cancer, lung, non- | CRT; Intetumum |
| | | | | | | small cell; Cancer, melanoma; Cancer, ovarian; | |
| | | | | | | Cancer, pancreatic; Cancer, peritoneal; Cancer, | |
| | | | | | | prostate; Cancer, renal; Cancer, sarcoma, | |
| | | | | | | general; Cancer, sarcoma, soft tissue; Cancer, | |
| | | | | | | solid, general; Diagnosis, cancer; Fibrosis, | |
| | | | | | | general; Fibrosis, pulmonary, idiopathic; | |
| | | | | | | Nephropathy, general | |
| JAK2 | Janus kinase 2 | Crohn's | 83 | rs10758669-C | Crohn's disease | Arthritis, rheumatoid; Cancer, breast; Cancer, | AC-430; AEG-41174; AG-490; AMG-Jak2-01; AT-9283; |
| | | disease | | (0.35) | | colorectal; Cancer, general; Cancer, | AZ-Tak1; AZD-1480; CYT-387; GLPG-0634; INCB-16562; |
| | | | | | | haematological, general; Cancer, head and | INCB-28050; LY-2784544; NS-018; ON-044580; SAR- |
| | | | | | | neck; Cancer, leukaemia, acute lymphocytic; | 302503; SB-1317; SB-1518; SGI-1252; Degrasyns; |
| | | | | | | Cancer, leukaemia, acute myelogenous; | Lestaurtinib; Ruxolitinib; Tozasertib lacta |
| | | | | | | Cancer, leukaemia, chronic myelogenous; | |
| | | | | | | Cancer, leukaemia, chronic myelomonocytic; | |
| | | | | | | Cancer, leukaemia, general; Cancer, liver; | |
| | | | | | | Cancer, lung, non-small cell; Cancer, | |
| | | | | | | lymphoma, B-cell; Cancer, lymphoma, | |
| | | | | | | Hodgkin's; Cancer, lymphoma, general; Cancer, | |
| | | | | | | lymphoma, non-Hodgkin's; Cancer, myeloma; | |
| | | | | | | Cancer, neuroendocrine, neuroblastoma; | |
| | | | | | | Cancer, ovarian; Cancer, pancreatic; Cancer, | |
| | | | | | | prostate; Cancer, solid, general; Hypertension, | |
| | | | | | | pulmonary; Idiopathic myelofibrosis; | |
| | | | | | | Myelodysplastic syndrome; Polycythaemia | |
| | | | | | | vera; Psoriasis; Thrombocythaemia; | |
| | | | | | | Thrombocytopenia, general; Thrombocytosis | |
| JAK2 | Janus kinase 2 | Myelo- | 1 | rs10974944-? | Myeloproliferative | Arthritis, rheumatoid; Cancer, breast; Cancer, | AC-430; AEG-41174; AG-490; AMG-Jak2-01; AT-9283; |
| | | proliferative | | (NR) | neoplasms | colorectal; Cancer, general; Cancer, | AZ-Tak1; AZD-1480; CYT-387; GLPG-0634; INCB-16562; |
| | | neoplasms | | | | haematological, general; Cancer, head and | INCB-28050; LY-2784544; NS-018; ON-044580; SAR- |
| | | | | | | neck; Cancer, leukaemia, acute lymphocytic; | 302503; SB-1317; SB-1518; SGI-1252; Degrasyns; |
| | | | | | | Cancer, leukaemia, acute myelogenous; | Lestaurtinib; Ruxolitinib; Tozasertib lacta |
| | | | | | | Cancer, leukaemia, chronic myelogenous; . | |
| | | | | | | Cancer, leukaemia, chronic myelomonocytic; | |
| | | | | | | Cancer, leukaemia, general; Cancer, liver; | |
| | | | | | | Cancer, lung, non-small cell; Cancer, | |
| | | | | | | lymphoma, B-cell; Cancer, lymphoma, | |
| | | | | | | Hodgkin's; Cancer, lymphoma, general; Cancer, | |
| | | | | | | lymphoma, non-Hodgkin's; Cancer, myeloma; | |
| | | | | | | Cancer, neuroendocrine, neuroblastoma; | |
| | | | | | | Cancer, ovarian; Cancer, pancreatic; Cancer, | |
| | | | | | | prostate; Cancer, solid, general; Hypertension, | |
| | | | | | | pulmonary; Idiopathic myelofibrosis; | |
| | | | | | | Myelodysplastic syndrome; Polycythaemia | |
| | | | | | | vera; Psoriasis; Thrombocythaemia; | |
| | | | | | | Thrombocytopenia, general; Thrombocytosis | |
| JAK2 | Janus kinase 2 | Ulcerative | 96 | rs10758669-C | Ulcerative colitis | Arthritis, rheumatoid; Cancer, breast; Cancer, | AC-430; AEG-41174; AG-490; AMG-Jak2-01; AT-9283; |
| | | colitis | | (0.35), | | colorectal; Cancer, general; Cancer, | AZ-Tak1; AZD-1480; CYT-387; GLPG-0634; INCB-16562; |
| | | | | rs10758669-C | | haematological, general; Cancer, head and | INCB-28050; LY-2784544; NS-018; ON-044580; SAR- |
| | | | | (NR), | | neck; Cancer, leukaemia, acute lymphocytic; | 302503; SB-1317; SB-1518; SGI-1252; Degrasyns; |
| | | | | rs10975003-C | | Cancer, leukaemia, acute myelogenous; | Lestaurtinib; Ruxolitinib; Tozasertib lacta |
| | | | | (0.19) | | Cancer, leukaemia, chronic myelogenous; | |
| | | | | | | Cancer, leukaemia, chronic myelomonocytic; | |
| | | | | | | Cancer, leukaemia, general; Cancer, liver; | |
| | | | | | | Cancer, lung, non-small cell; Cancer, | |
| | | | | | | lymphoma, B-cell; Cancer, lymphoma, | |
| | | | | | | Hodgkin's; Cancer, lymphoma, general; Cancer, | |
| | | | | | | lymphoma, non-Hodgkin's; Cancer, myeloma; | |
| | | | | | | Cancer, neuroendocrine, neuroblastoma; | |
| | | | | | | Cancer, ovarian; Cancer, pancreatic; Cancer, | |
| | | | | | | prostate; Cancer, solid, general; Hypertension, | |
| | | | | | | pulmonary; Idiopathic myelofibrosis; | |
| | | | | | | Myelodysplastic syndrome; Polycythaemia | |
| | | | | | | vera; Psoriasis; Thrombocythaemia; | |
| | | | | | | Thrombocytopenia, general; Thrombocytosis | |
| JUN | jun proto- | Cognitive | 39 | rs4601609-? | Cognitive | Arthritis, osteo; Arthritis, rheumatoid; Cancer, | DNAzymes, NSI; Dz-13; T-5224; Tarenflurbil, PAZ; |
| | oncogene | performance | | (0.05) | performance | basal cell; Cancer, breast; Cancer, prostate; | Trilosta |
| | | | | | | Cancer, skin, general; Cushing's disease; Heart | |
| | | | | | | failure; Macular degeneration, age-related, | |
| | | | | | | general; Pain, general; Pain, neuropathic; | |
| | | | | | | Restenosis; Retinopathy, diabetic | |
| KCNMA1 | potassium large | Mortality | 10 | rs4979906-G | Mortality | Glaucoma; Impotence; Incontinence, urinary; | NS-1619; NS-8; HMaxi-K; Isopropyl unoprosto |
| | conductance | among heart | | (0.186) | among heart | Macular degeneration, age-related, dry; | |
| | calcium-activated | failure | | | failure patients | Oedema, macular, diabetic; Overactive | |
| | channel, | patients | | | | bladder; Pollakisuria; Psychosis, general; | |
| | subfamily M, | | | | | Retinitis pigmentosa | |
| | alpha member 1 | | | | | | |
| KCNQ1 | potassium | Type 2 | 61 | rs2237892-C | Type 2 diabetes | Fibrillation, atrial; Fibrillation, ventricular; | Azimilide dihydrochlori |
| | voltage-gated | diabetes | | (0.59), | | Tachycardia, supraventricular | |
| | channel, KQT-like | | | rs2237892-C | | | |
| | subfamily, | | | (0.61), | | | |
| | member 1 | | | rs2237892-C | | | |
| | | | | (NR), | | | |
| | | | | rs2237895-C | | | |
| | | | | (0.33), | | | |
| | | | | rs2237897-C | | | |
| | | | | (0.34), | | | |
| | | | | rs231362-G | | | |
| | | | | (NR) | | | |
| KIF11 | kinesin family | Type 2 | 61 | rs6583826-G | Type 2 diabetes | Cancer, bladder; Cancer, breast; Cancer, | 45C-205; ALN-VSP; ARQ-621; ARRY-520; AZD-4877; SB- |
| | member 11 | diabetes | | (0.26) | | colorectal; Cancer, gastrointestinal, stomach; | 743921; Ispinesib mesylate; Litrones |
| | | | | | | Cancer, head and neck; Cancer, leukaemia, | |
| | | | | | | acute lymphocytic; Cancer, leukaemia, acute | |
| | | | | | | myelogenous; Cancer, leukaemia, chronic | |
| | | | | | | lymphocytic; Cancer, leukaemia, chronic | |
| | | | | | | myelogenous; Cancer, leukaemia, chronic | |
| | | | | | | myelomonocytic; Cancer, liver; Cancer, lung, | |
| | | | | | | non-small cell; Cancer, lung, small cell; Cancer, | |
| | | | | | | lymphoma, B-cell; Cancer, lymphoma, | |
| | | | | | | Hodgkin's; Cancer, lymphoma, T-cell; Cancer, | |
| | | | | | | lymphoma, general; Cancer, lymphoma, non- | |
| | | | | | | Hodgkin's; Cancer, melanoma; Cancer, | |
| | | | | | | myeloma; Cancer, neuroendocrine, pancreatic; | |
| | | | | | | Cancer, oesophageal; Cancer, ovarian; Cancer, | |
| | | | | | | pancreatic; Cancer, prostate; Cancer, renal; | |
| | | | | | | Cancer, solid, general; Cancer, squamous cell; | |
| | | | | | | Myelodysplastic syndrome | |
| KIT | v-kit Hardy- | Bipolar | 21 | rs2537859-T | Bipolar disorder | Alzheimer's disease; Anaemia, | AMG-191; DCC-2157; DCC-2618; DP-2514; ENMD- |
| | Zuckerman 4 | disorder | | (0.60) | | radio/chemotherapy-induced; Arthritis, | 2076; KIT816 inhibitor, AB Science; 051-930; PLX-3397; |
| | feline sarcoma | | | | | rheumatoid; Asthma; Cancer, biliary; Cancer, | PLX-647; SU-14813; XL-820; XL-999; Amuvatinib; |
| | viral oncogene | | | | | bladder; Cancer, brain; Cancer, breast; Cancer, | Ancestim; Axitinib; Cabozantinib; Cediranib; Dasatinib; |
| | homolog | | | | | cervical; Cancer, colorectal; Cancer, | Dovitinib lactate; Foretinib; Imatinib mesilate; |
| | | | | | | endometrial; Cancer, fallopian tube; Cancer, | Lenvatinib; Masitinib; Midostaurin; Motesanib |
| | | | | | | gastrointestinal, general; Cancer, | diphosphate; Nilotinib; Pazopanib hydrochloride; |
| | | | | | | gastrointestinal, stomach; Cancer, | Pazopanib hydrochloride (ophthalmic); Regorafenib; |
| | | | | | | gastrointestinal, stromal; Cancer, | Sunitinib malate; Tandutinib; Tivozanib; Vatalan |
| | | | | | | haematological, general; Cancer, head and | |
| | | | | | | neck; Cancer, leukaemia, acute lymphocytic; | |
| | | | | | | Cancer, leukaemia, acute myelogenous; | |
| | | | | | | Cancer, leukaemia, chronic lymphocytic; | |
| | | | | | | Cancer, leukaemia, chronic myelogenous; | |
| | | | | | | Cancer, leukaemia, mast cell; Cancer, liver; | |
| | | | | | | Cancer, lung, non-small cell; Cancer, lung, small | |
| | | | | | | cell; Cancer, lymphoma, Hodgkin's; Cancer, | |
| | | | | | | lymphoma, T-cell; Cancer, lymphoma, general; | |
| | | | | | | Cancer, lymphoma, non-Hodgkin's; Cancer, | |
| | | | | | | melanoma; Cancer, mesothelioma; Cancer, | |
| | | | | | | myeloma; Cancer, nasopharyngeal; Cancer, | |
| | | | | | | neuroendocrine, carcinoid; Cancer, | |
| | | | | | | neuroendocrine, general; Cancer, | |
| | | | | | | neuroendocrine, pancreatic; Cancer, | |
| | | | | | | oesophageal; Cancer, ovarian; Cancer, | |
| | | | | | | pancreatic; Cancer, peritoneal; Cancer, | |
| | | | | | | prostate; Cancer, renal; Cancer, sarcoma, | |
| | | | | | | general; Cancer, sarcoma, glial; Cancer, | |
| | | | | | | sarcoma, leiomyo; Cancer, sarcoma, soft | |
| | | | | | | tissue; Cancer, sarcoma, synovial; Cancer, solid, | |
| | | | | | | general; Cancer, testicular; Cancer, thyroid; | |
| | | | | | | Fibrosis, pulmonary; Fibrosis, pulmonary, | |
| | | | | | | idiopathic; Hypereosinophilic syndrome, | |
| | | | | | | idiopathic; Hypertension, pulmonary; | |
| | | | | | | Inflammation, general; Macular degeneration, | |
| | | | | | | age-related, wet; Mastocytosis; | |
| KIT | v-kit Hardy- | Bipolar | 21 | rs2537859-T | Bipolar disorder | Multiple sclerosis, general; Multiple sclerosis, | AMG-191; DCC-2157; DCC-2618; DP-2514; ENMD- |
| continued | Zuckerman 4 | disorder | | (0.60) | | progressive, primary; Multiple sclerosis, | 2076; KIT816 inhibitor, AB Science; OSI-930; PLX-3397; |
| | feline sarcoma | | | | | progressive, secondary; Myelodysplastic | PLX-647; SU-14813; XL-820; XL-999; Amuvatinib; |
| | viral oncogene | | | | | syndrome; Oedema, macular, diabetic; | Ancestim; Axitinib; Cabozantinib; Cediranib; Dasatinib; |
| | homolog | | | | | Polycythaemia vera; Psoriasis; Scleroderma; | Dovitinib lactate; Foretinib; Imatinib mesilate; |
| | | | | | | Stem cell mobilization; Transplant rejection, | Lenvatinib; Masitinib; Midostaurin; Motesanib |
| | | | | | | bone marrow | diphosphate; Nilotinib; Pazopanib hydrochloride; |
| | | | | | | | Pazopanib hydrochloride (ophthalmic); Regorafenib; |
| | | | | | | | Sunitinib malate; Tandutinib; Tivozanib; Vatalan |
| LAMC2 | laminin, gamma 2 | Systemic | 42 | rs525410-? | Systemic lupus | Cancer, breast; Cancer, colorectal; Cancer, | CYN-1 |
| | | lupus | | (0.49) | erythematosus | lung, non-small cell; Cancer, ovarian; Cancer, | |
| | | erythematosus | | | | pancreatic | |
| LAMC2 | laminin, gamma 2 | Coronary | 78 | rs1028771-A | Coronary heart | Cancer, breast; Cancer, colorectal; Cancer, | CYN-1 |
| | | heart disease | | (0.97) | disease | lung, non-small cell; Cancer, ovarian; Cancer, | |
| | | | | | | pancreatic | |
| LCAT | lecithin- | Coronary | 78 | rs3729639-T | Coronary heart | Atherosclerosis | AAV8-hLCAT, Merck & |
| | cholesterol | heart disease | | (0.44) | disease | | |
| | acyltransferase | | | | | | |
| LDLR | low density | Coronary | 78 | rs1122608-G | Coronary heart | Atherosclerosis; Cystic fibrosis; | COR-2; LDL-4X4; LDLr PLASmin DNA complexes; |
| | lipoprotein | heart disease | | (0.77) | disease | Hypercholesterolaemia; Hyperlipidaemia, | Pharmaprojects No. 5637; Axitirome; Gene therapy, |
| | receptor | | | | | general | Geno |
| LINGO1 | leucine rich | Essential | 1 | rs9652490-G | Essential tremor | Multiple sclerosis, general; Multiple sclerosis, | BIIB-033; Li33 PEG-Fab, Biogen Id |
| | repeat and Ig | tremor | | (0.23) | | progressive, secondary; Multiple sclerosis, | |
| | domain | | | | | relapsing-remitting | |
| | containing 1 | | | | | | |
| LIPA | lipase A, | Coronary | 78 | rs1412444-T | Coronary heart | Atherosclerosis; Lysosomal acid lipase | Lysosomal acid lipase, Large S; Recombinant lysosomal |
| | lysosomal acid, | heart disease | | (0.32), | disease | deficiency | acid lipase, Synageva BioPhar |
| | cholesterol | | | rs1412444-T | | | |
| | esterase | | | (0.42) | | | |
| LPAR3 | lysophosphatidic | Type 1 | 50 | rs1983853-? | Type 1 diabetes | Fibrosis, general | SAR-1008 |
| | acid receptor 3 | diabetes | | (NR) | | | |
| LPL | lipoprotein lipase | HDL | 6 | rs13702-A | HDL Cholesterol- | Atherosclerosis; Bursitis; Cystic fibrosis; | Vessiflex; Alipogene tiparvovec; Bezafibrate; |
| | | Cholesterol- | | (NR) | Triglycerides | Diabetes, Type 2; Diabetes, general; Diabetic | Binifibrate; Ciprofibrate; Docosanol; Eniclobrate; |
| | | Triglycerides | | | | complication, general; Hypercholesterolaemia; | Etofibrate; Gemfibrozil; Pancreatin, Solvay; |
| | | | | | | Hyperlipidaemia, general; Infarction, | Sulodexide, Alfa Wassermann; Sulodexide, Ker |
| | | | | | | myocardial; Infection, herpes simplex virus; | |
| | | | | | | Inflammation, vascular; Nephropathy, diabetic; | |
| | | | | | | Pancreatic dysfunction, general; Retinopathy, | |
| | | | | | | diabetic; Steatohepatitis; Thrombosis, general; | |
| | | | | | | Venous insufficiency | |
| LPL | lipoprotein lipase | Triglycerides- | 6 | rs15285-A | Triglycerides- | Atherosclerosis; Bursitis; Cystic fibrosis; | Vessiflex; Alipogene tiparvovec; Bezafibrate; |
| | | Blood | | (NR) | Blood Pressure | Diabetes, Type 2; Diabetes, general; Diabetic | Binifibrate; Ciprofibrate; Docosanol; Eniclobrate; |
| | | Pressure | | | | complication, general; Hypercholesterolaemia; | Etofibrate; Gemfibrozil; Pancreatin, Solvay; |
| | | | | | | Hyperlipidaemia, general; Infarction, | Sulodexide, Alfa Wassermann; Sulodexide, Ker |
| | | | | | | myocardial; Infection, herpes simplex virus; | |
| | | | | | | Inflammation, vascular; Nephropathy, diabetic; | |
| | | | | | | Pancreatic dysfunction, general; Retinopathy, | |
| | | | | | | diabetic; Steatohepatitis; Thrombosis, general; | |
| | | | | | | Venous insufficiency | |
| LPL | lipoprotein lipase | Hyper- | 5 | rs7016880-? | Hypertriglyceridemia | Atherosclerosis; Bursitis; Cystic fibrosis; | Vessiflex; Alipogene tiparvovec; Bezafibrate; |
| | | triglyceridemia | | (0.90) | | Diabetes, Type 2; Diabetes, general; Diabetic | Binifibrate; Ciprofibrate; Docosanol; Eniclobrate; |
| | | | | | | complication, general; Hypercholesterolaemia; | Etofibrate; Gemfibrozil; Pancreatin, Solvay; |
| | | | | | | Hyperlipidaemia, general; Infarction, | Sulodexide, Alfa Wassermann; Sulodexide, Ker |
| | | | | | | myocardial; Infection, herpes simplex virus; | |
| | | | | | | Inflammation, vascular; Nephropathy, diabetic; | |
| | | | | | | Pancreatic dysfunction, general; Retinopathy, | |
| | | | | | | diabetic; Steatohepatitis; Thrombosis, general; | |
| | | | | | | Venous insufficiency | |
| LRP1 | low density | Migraine | 7 | rs11172113-T | Migraine | Cancer, liver; Infection, hepatitis virus, | HepTide; Antiviral, HepTide; Doxorubicin, HepTi |
| | lipoprotein | | | (0.59) | | unspecified; Not applicable | |
| | receptor-related | | | | | | |
| | protein 1 | | | | | | |
| LRRK2 | leucine-rich | Crohn's | 83 | rs11175593-T | Crohn's disease | Alzheimer's disease; Cancer, general; | LRRK2 inhibitors, Vernalis; LRRK2 inhibitors, Zenobia; |
| | repeat kinase 2 | disease | | (0.02), | | Parkinson's disease | TTT-30 |
| | | | | rs11564258-A | | | |
| | | | | (0.03) | | | |
| LRRK2 | leucine-rich | Parkinson's | 47 | rs1491942-G | Parkinson's | Alzheimer's disease; Cancer, general; | LRRK2 inhibitors, Vernalis; LRRK2 inhibitors, Zenobia; |
| | repeat kinase 2 | disease | | (0.08), | disease | Parkinson's disease | TTT-30 |
| | | | | rs1994090-? | | | |
| | | | | (NR) | | | |
| LTA | lymphotoxin | Neonatal | 10 | rs3099844-? | Neonatal lupus | Arthritis, rheumatoid; Cancer, oesophageal | 53550; RG-7416; Recombinant human lymphotoxin- |
| | alpha (TNF | lupus | | (0.11) | | | alpha derivative, Fudan-Zhangjia |
| | superfamily, | | | | | | |
| | member 1) | | | | | | |
| LY75 | lymphocyte | Nephropathy | 23 | rs4664308-? | Nephropathy | Cancer, bladder; Cancer, breast; Cancer, lung, | CDX-1401; CDX-24 |
| | antigen 75 | | | (0.43) | | non-small cell; Cancer, melanoma; Cancer, | |
| | | | | | | myeloma; Cancer, ovarian; Cancer, sarcoma, | |
| | | | | | | osteo; Cancer, solid, general; Infection, HIV | |
| | | | | | | prophylaxis; Infection, HIV/AIDS | |
| MAPT | microtubule- | Parkinson's | 47 | rs11012-T | Parkinson's | Alzheimer's disease; Cancer, general; | Alzheimer's MAb, AC Immune-2; Alzheimer's disease |
| | associated | disease | | (NR), | disease | Diagnosis, CNS; Parkinson's disease | therapy, TauRx; Alzheimer's disease therapy, |
| | protein tau | | | rs199533-C | | | Treventis; Alzheimer's imaging agent, Avid; |
| | | | | (0.78), | | | Alzheimer's therapy, Oligomerix; BLV-0703; LMT-X; |
| | | | | rs2942168-G | | | ReS10-T; ReS19-T; ReS3-T; ReS8-T; TTT-3002; |
| | | | | (0.78), | | | Methylthioninium chloride, TauR; Tau vaccine, AC |
| | | | | rs393152-A | | | Immu |
| | | | | (0.82), | | | |
| | | | | rs8070723-? | | | |
| | | | | (0.76) | | | |
| MC1R | melanocortin 1 | Melanoma | 4 | rs258322-A | Melanoma | Acne; Albinism; Arthritis, rheumatoid; Cancer, | AP-1030; AP-1189; AP-214; AP-405; JNJ-10229570; |
| | receptor (alpha | | | (0.09), | | melanoma; Cancer, squamous cell; Diabetes, | ME-10393; MSH fusion toxin; Afamelanotide; DRI- |
| | melanocyte | | | rs4785763-A | | Type 2; Eczema, atopic; Erythropoetic | alpha-MSH, Sano |
| | stimulating | | | (0.32) | | protoporphyria; Inflammatory bowel disease, | |
| | hormone | | | | | general; Insulin-related metabolic syndrome; | |
| | receptor) | | | | | Keratosis, actinic; Obesity; Photodamage; | |
| | | | | | | Polymorphous light eruption; Psoriasis; Renal | |
| | | | | | | failure; Respiratory distress syndrome, adult; | |
| | | | | | | Urticaria; Uveitis; Vitiligo | |
| MET | met proto- | Multiple | 59 | rs10243024-? | Multiple | Acute lung injury; Cancer, biliary; Cancer, | BAY-85-3474; BB-3; HuMax-cMet; INCB-028060; JNJ- |
| | oncogene | sclerosis | | (0.23) | sclerosis | bladder; Cancer, brain; Cancer, breast; Cancer, | 38877605; LY-2875358; MGCD-265; PF-4217903; |
| | (hepatocyte | | | | | cervical; Cancer, colorectal; Cancer, | Cabozantinib; Crizotinib; Ficlatuzumab; Foretinib; |
| | growth factor | | | | | endometrial; Cancer, fallopian tube; Cancer, | Golvatinib; Hepatocyte growth factor, ChronTech |
| | receptor) | | | | | gastrointestinal, stomach; Cancer, head and | Pharma; Onartuzumab; Rilotumumab; Tivantin |
| | | | | | | neck; Cancer, liver; Cancer, lung, general; | |
| | | | | | | Cancer, lung, non-small cell; Cancer, lung, small | |
| | | | | | | cell; Cancer, lymphoma, Hodgkin's; Cancer, | |
| | | | | | | lymphoma, T-cell; Cancer, lymphoma, general; | |
| | | | | | | Cancer, lymphoma, non-Hodgkin's; Cancer, | |
| | | | | | | melanoma; Cancer, myeloma; Cancer, | |
| | | | | | | neuroendocrine, Merkel cell carcinoma; | |
| | | | | | | Cancer, neuroendocrine, carcinoid; Cancer, | |
| | | | | | | neuroendocrine, general; Cancer, oesophageal; | |
| | | | | | | Cancer, oral; Cancer, ovarian; Cancer, | |
| | | | | | | pancreatic; Cancer, peritoneal; Cancer, | |
| | | | | | | prostate; Cancer, renal; Cancer, sarcoma, | |
| | | | | | | general; Cancer, sarcoma, glial; Cancer, | |
| | | | | | | sarcoma, lipo; Cancer, sarcoma, soft tissue; | |
| | | | | | | Cancer, solid, general; Cancer, squamous cell; | |
| | | | | | | Cancer, testicular; Cancer, thyroid; Cancer, | |
| | | | | | | vaginal; Fibrosis, liver; Heart failure; Infarction, | |
| | | | | | | myocardial; Renal failure; Spinal cord injury; | |
| | | | | | | Transplant rejection, general; Ulcer, general; | |
| | | | | | | Wound healing | |
| MTNR1A | melatonin | Obesity | 29 | rs925642-? | Obesity | Addiction, tranquillizer; Alzheimer's disease; | GW-290569; NCT-600; SL-18.1616; Agomelatine; |
| | receptor 1A | | | (NR) | | Apnoea; Depression, general; Depression, | Buspirone + melatonin, BrainCells; Melatonin, Neurim; |
| | | | | | | major depressive disorder; Dyskinesia, tardive; | Ramelteon; Tasimelte |
| | | | | | | Generalized anxiety disorder; Insomnia; | |
| | | | | | | Migraine prophylaxis; Obsessive-compulsive | |
| | | | | | | disorder; Sleep disorder, general | |
| MTNR1B | melatonin | Fasting | 6 | rs10830963-G | Fasting plasma | Addiction, tranquillizer; Alzheimer's disease; | GW-290569; NCT-600; SL-18.1616; Agomelatine; |
| | receptor 1B | plasma | | (0.28), | glucose | Apnoea; Depression, general; Depression, | Buspirone + melatonin, BrainCells; Melatonin, Neurim; |
| | | glucose | | rs1387153-T | | major depressive disorder; Dyskinesia, tardive; | Ramelteon; Tasimelte |
| | | | | (0.29), | | Generalized anxiety disorder; Insomnia; | |
| | | | | rs2166706-G | | Migraine prophylaxis; Obsessive-compulsive | |
| | | | | (0.46) | | disorder; Sleep disorder, general | |
| MTNR1B | melatonin | Type 2 | 61 | rs1387153-T | Type 2 diabetes | Addiction, tranquillizer; Alzheimer's disease; | GW-290569; NCT-600; SL-18.1616; Agomelatine; |
| | receptor 1B | diabetes | | (NR) | | Apnoea; Depression, general; Depression, | Buspirone + melatonin, BrainCells; Melatonin, Neurim; |
| | | | | | | major depressive disorder; Dyskinesia, tardive; | Ramelteon; Tasimelte |
| | | | | | | Generalized anxiety disorder; Insomnia; | |
| | | | | | | Migraine prophylaxis; Obsessive-compulsive | |
| | | | | | | disorder; Sleep disorder, general | |
| MTNR1B | melatonin | Fasting | 18 | rs10830963-G | Fasting glucose- | Addiction, tranquillizer; Alzheimer's disease; | GW-290569; NCT-600; SL-18.1616; Agomelatine; |
| | receptor 1B | glucose- | | (0.30) | related traits | Apnoea; Depression, general; Depression, | Buspirone + melatonin, BrainCells; Melatonin, Neurim; |
| | | related traits | | | | major depressive disorder; Dyskinesia, tardive; | Ramelteon; Tasimelte |
| | | | | | | Generalized anxiety disorder; Insomnia; | |
| | | | | | | Migraine prophylaxis; Obsessive-compulsive | |
| | | | | | | disorder; Sleep disorder, general | |
| MUC1 | mucin 1, cell | Crohn's | 83 | rs3180018-A | Crohn's disease | Cancer, bladder; Cancer, breast; Cancer, | AS-109; AS-1402; AS-1403; BrevaRex MAb; CMB-401; |
| | surface | disease | | (0.25) | | colorectal; Cancer, fallopian tube; Cancer, | CVac; GO-203-2c; GT-MAB 2.5-GEX; R-1549; SAR- |
| | associated | | | | | gastrointestinal, general; Cancer, general; | 566658; TG-4010; Emepepimut-S; Falimarev + |
| | | | | | | Cancer, head and neck; Cancer, liver; Cancer, | inalimarev; HPAM4-SN-38; ImMucin; Mannan-MUC1, |
| | | | | | | lung, general; Cancer, lung, non-small cell; | Viralytics; Yttrium Y 90 clivatuzumab tetraxet |
| | | | | | | Cancer, myeloma; Cancer, oesophageal; | |
| | | | | | | Cancer, ovarian; Cancer, pancreatic; Cancer, | |
| | | | | | | peritoneal; Cancer, prostate; Cancer, renal; | |
| | | | | | | Cancer, solid, general; Diagnosis, cancer | |
| MYC | v-myc | Ovarian | 10 | rs10088218-G | Ovarian cancer | Cancer, breast; Cancer, general; Cancer, head | AVI-4126; INXC-6295; Degrasy |
| | myelocytomatosis | cancer | | (NR) | | and neck; Cancer, prostate; Polycystic kidney | |
| | viral oncogene | | | | | disease; Restenosis | |
| | homolog (avian) | | | | | | |
| NAGLU | N- | Conduct | 17 | rs7581919-? | Conduct | Mucopolysaccharidosis IIIB | 623 |
| | acetylglucosaminidase, | disorder | | (0.041) | disorder | | |
| | alpha | | | | | | |
| NCAM1 | neural cell | Left | 3 | rs1436109-? | Left ventricular | Alzheimer's disease; Cancer, brain; Cancer, | EMD-56700; ERIC-1; PR-21, Pharmaxon; HuN901-DC1; |
| | adhesion | ventricular | | (NR) | mass | cervical; Cancer, gastrointestinal, general; | Lorvotuzumab mertansi |
| | molecule 1 | mass | | | | Cancer, lung, small cell; Cancer, myeloma; | |
| | | | | | | Cancer, neuroendocrine, Merkel cell | |
| | | | | | | carcinoma; Cancer, neuroendocrine, general; | |
| | | | | | | Cancer, ovarian; Cancer, sarcoma, general; | |
| | | | | | | Cancer, skin, general; Cancer, solid, general; | |
| | | | | | | Cognitive disorder, unspecified; Spinal cord | |
| | | | | | | injury | |
| NEDD4 | neural precursor | Keloid | 2 | rs8032158-C | Keloid | Infection, influenza virus prophylaxis | FGI-1 |
| | cell expressed, | | | (0.36) | | | |
| | developmentally | | | | | | |
| | down-regulated 4 | | | | | | |
| NEDD4 | neural precursor | Chronic | 17 | rs7169431-A | Chronic | Infection, influenza virus prophylaxis | FGI-1 |
| | cell expressed, | lymphocytic | | (NR) | lymphocytic | | |
| | developmentally | leukemia | | | leukemia | | |
| | down-regulated 4 | | | | | | |
| NFKB1 | nuclear factor of | Primary | 26 | rs7665090-C | Primary biliary | Acute coronary syndrome; Amyloidosis; | 7-Hydroxyfrullanolide, Piramal; ALT-2074; HE-3286; |
| | kappa light | biliary | | (0.52) | cirrhosis | Arthritis, osteo; Arthritis, rheumatoid; Asthma; | MLN-9708; NFkB decoy gene ther, Dainippon; NFkB |
| | polypeptide gene | cirrhosis | | | | Cancer, biliary; Cancer, bladder; Cancer, brain; | decoy oligo, AnGes; OT-440; P-1639; Bardoxolone; |
| | enhancer in B- | | | | | Cancer, breast; Cancer, colorectal; Cancer, | Bardoxolone methyl; Bortezomib; Declopramide; |
| | cells 1 | | | | | endometrial; Cancer, gastrointestinal, general; | Quinacrine, Cleveland; Tarenflurbil, PAZ; Teglarinad |
| | | | | | | Cancer, head and neck; Cancer, leukaemia, | chlori |
| | | | | | | acute lymphocytic; Cancer, leukaemia, acute | |
| | | | | | | myelogenous; Cancer, leukaemia, chronic | |
| | | | | | | lymphocytic; Cancer, leukaemia, chronic | |
| | | | | | | myelogenous; Cancer, liver; Cancer, lung, non- | |
| | | | | | | small cell; Cancer, lung, small cell; Cancer, | |
| | | | | | | lymphoma, B-cell; Cancer, lymphoma, | |
| | | | | | | Hodgkin's; Cancer, lymphoma, general; Cancer, | |
| | | | | | | lymphoma, non-Hodgkin's; Cancer, melanoma; | |
| | | | | | | Cancer, myeloma; Cancer, neuroendocrine, | |
| | | | | | | carcinoid; Cancer, oesophageal; Cancer, | |
| | | | | | | ovarian; Cancer, pancreatic; Cancer, prostate; | |
| | | | | | | Cancer, renal; Cancer, sarcoma, osteo; Cancer, | |
| | | | | | | sarcoma, soft tissue; Cancer, solid, general; | |
| | | | | | | Cancer, thyroid; Chronic obstructive pulmonary | |
| | | | | | | disease; Colitis, ulcerative; Diabetes, Type 2; | |
| | | | | | | Eczema, atopic; Glaucoma; | |
| | | | | | | Glomerulonephritis; Hepatitis, non-infectious; | |
| | | | | | | Inflammation, general; Inflammatory bowel | |
| | | | | | | disease, general; Ischaemia, cerebral; | |
| | | | | | | Mucositis, general; Myelodysplastic syndrome; | |
| | | | | | | Nephropathy, diabetic; Pain, general; Pain, | |
| | | | | | | neuropathic; Parkinson's disease; Psoriasis; | |
| | | | | | | Regeneration, cartilage; Renal failure; | |
| | | | | | | Restenosis; Transplant rejection, bone marrow; | |
| | | | | | | Waldenstrom's hypergammaglobulinaemia | |
| NOS2 | nitric oxide | Psoriasis | 30 | rs4795067-G | Psoriasis | Arthritis, osteo; Arthritis, rheumatoid; Head | CR-3294; KD-7332; KLYP-956; PMI-005; SD-6010; VAS- |
| | synthase 2, | | | (0.35) | | trauma; Hypotension; Inflammatory bowel | 203; INOS gene therapy, GenVec; NNOS/iNOS |
| | inducible | | | | | disease, general; Ischaemia, cerebral; | inhibitors, NeurAxon; Targini |
| | | | | | | Migraine; Mucositis, general; Pain, general; | |
| | | | | | | Pain, neuropathic; Radio/chemotherapy- | |
| | | | | | | induced injury, GI; Radio/chemotherapy- | |
| | | | | | | induced mucositis; Restenosis; Sepsis | |
| NR4A2 | nuclear receptor | Response to | 75 | rs16839962-T | Response to | Parkinson's disease | Nurr1-RXR agonists, ACAD |
| | subfamily 4, | statin therapy | | (0.13) | statin therapy | | |
| | group A, member 2 | | | | | | |
| NRG1 | neuregulin 1 | Dialysis- | 31 | rs2439312-? | Dialysis-related | Heart failure; Multiple sclerosis, general | Glial growth factor-2, Paion; RhNRG |
| | | related | | (0.24) | mortality | | |
| | | mortality | | | | | |
| NRG1 | neuregulin 1 | Hirschsprung's | 4 | rs16879552-G | Hirschsprung's | Heart failure; Multiple sclerosis, general | Glial growth factor-2, Paion; RhNRG |
| | | disease | | (0.61) | disease | | |
| NRG1 | neuregulin 1 | Hip geometry | 7 | rs10503887-? | Hip geometry | Heart failure; Multiple sclerosis, general | Glial growth factor-2, Paion; RhNRG |
| | | | | (NR) | | | |
| NRP1 | neuropilin 1 | Schizophrenia | 33 | rs1412115-? | Schizophrenia | Cancer, ovarian; Cancer, prostate; Cancer, | MNRP-1685A; RG-73 |
| | | | | (0.37) | | solid, general | |
| OPRM1 | opioid receptor, | Coronary | 78 | rs675026-? | Coronary heart | Addiction, alcohol; Addiction, cocaine; | 57138; 58209; 59760; 9624; ADL-5510; ADL-5945; |
| | mu 1 | heart disease | | (0.72) | disease | Addiction, drug, unspecified; Addiction, | ADL-7445; ALKS-33; ALKS-36; ALKS-5461; Cyt-1010; |
| | | | | | | gambling; Addiction, narcotic/opiate; | DPI-3290; EP-94; GSK-1521498; HydrocoDex; JNJ- |
| | | | | | | Addiction, nicotine; Anaesthesia; Anaesthesia, | 27018966; KIN-3031; KIN-4044; LY-255582; NCT-400; |
| | | | | | | adjunct; Binge eating disorder; Bulimia; Cancer, | NKP-206; NKTR-119; NKTR-181; NRP-290; NRT-300; |
| | | | | | | lung, non-small cell; Constipation; Depression, | PTI-609; Penntuss; PercoDex; SYN-1003; TD-1211; |
| | | | | | | general; Depression, major depressive | Acetaminophen + hydrocodone, Qu; Acetaminophen + |
| | | | | | | disorder; Dyskinesia, levodopa-induced; Ileus; | propoxyphene, Xanodyne; Acetaminophen + |
| | | | | | | Infarction, myocardial; Irritable bowel | tramadol, Ethypharm; Alfentanil; Alvimopan; |
| | | | | | | syndrome; Migraine; Motility dysfunction, GI, | Buprenorphine; Buprenorphine + naloxone; |
| | | | | | | general; Nausea and vomiting, opiate-induced; | Buprenorphine + naloxone, Aoxing; Buprenorphine + |
| | | | | | | Neuropathy, diabetic; Obesity; Pain, cancer; | naloxone, BDSI; Buprenorphine + naloxone, |
| | | | | | | Pain, general; Pain, musculoskeletal, arthritis; | Nanotherapeutics; Buprenorphine + naloxone, Orexo; |
| | | | | | | Pain, musculoskeletal, general; Pain, | Buprenorphine, BEMA film (low dose); Buprenorphine, |
| | | | | | | neuropathic; Pain, post-herpetic; Pain, post- | Camurus; Buprenorphine, Elbion; Buprenorphine, |
| | | | | | | operative; Poisoning, drug; Premature | Encore; Buprenorphine, Gruenenthal; Buprenorphine, |
| | | | | | | ejaculation; Pruritus; Restless legs syndrome; | Labtec; Buprenorphine, Mundipharma; |
| | | | | | | Rhinitis, allergic, general; Rhinitis, general; | Buprenorphine, ProNeura; Buprenorphine, Vernalis; |
| | | | | | | Sepsis; Urinary retention | Buprenorphine, Vyteris; Buprenorphine, sublingual; |
| | | | | | | | Bupropion + naltrexone, Orexigen; Butorphanol |
| | | | | | | | tartrate; Butorphanol, MDRNA; Chiral methadones, |
| | | | | | | | SAF; Dexketoprofen trometamol + tramadol |
| | | | | | | | hydrochloride, Menarini; Dextropropoxyphene, |
| | | | | | | | Benzon; Dihydrocodeine, Mundipharma; Fentanyl |
| | | | | | | | (sublingual), INSYS; Fentanyl citrate, Altea; Fentanyl |
| | | | | | | | citrate, Anesta; Fentanyl citrate, Glide; Fentanyl |
| | | | | | | | citrate, Hisamitsu; Fentanyl citrate, Orexo; Fentanyl |
| | | | | | | | film, Auxilium; Fentanyl patch, Nippon Kayaku; |
| | | | | | | | Fentanyl, Acrux; Fentanyl, Alexza; Fentanyl, Alza; |
| | | | | | | | Fentanyl, Archimedes; Fentanyl, BioAlliance Pharma; |
| | | | | | | | Fentanyl, CIMA; Fentanyl, DELEX; Fentanyl, E-TRANS; |
| | | | | | | | Fentanyl, Ethypharm; Fentanyl, Lavipharm; Fentanyl, |
| | | | | | | | Meda; Fentanyl, NAL Pharma-3; Fentanyl, Taifun; |
| | | | | | | | Fentanyl, Watson; Fentanyl, nasal, Nycomed; |
| OPRM1 | opioid receptor, | Coronary | 78 | rs675026-? | Coronary heart | Addiction, alcohol; Addiction, cocaine; | Fentanyl, transdermal, Dr Reddy's; Fluoxetine + |
| continued | mu 1 | heart disease | | (0.72) | disease | Addiction, drug, unspecified; Addiction, | naltrexone, Orex; Flupirtine + opioid, CNSBio; |
| | | | | | | gambling; Addiction, narcotic/opiate; | Hydrocodone + acetaminophen + niacin, Acura; |
| | | | | | | Addiction, nicotine; Anaesthesia; Anaesthesia, | Hydrocodone + acetaminophen, Abbott; Hydrocodone + |
| | | | | | | adjunct; Binge eating disorder; Bulimia; Cancer, | ibuprofen, ProEth; Hydrocodone + |
| | | | | | | lung, non-small cell; Constipation; Depression, | ibuprofen, Watson; Hydrocodone + naltrexone, King; |
| | | | | | | general; Depression, major depressive | Hydrocodone + promethazine + acetaminophen; |
| | | | | | | disorder; Dyskinesia, levodopa-induced; Ileus; | Hydrocodone bitartrate, Cephalon; |
| | | | | | | Infarction, myocardial; Irritable bowel | Hydrocodone + acetaminophen, Vic; Hydrocodone, |
| | | | | | | syndrome; Migraine; Motility dysfunction, GI, | Egalet; Hydrocodone, Egalet-2; Hydrocodone, |
| | | | | | | general; Nausea and vomiting, opiate-induced; | Inspirion Delivery Technologies; Hydromorphone HCl, |
| | | | | | | Neuropathy, diabetic; Obesity; Pain, cancer; | OROS; Hydromorphone, CR, Egalet; Hydromorphone, |
| | | | | | | Pain, general; Pain, musculoskeletal, arthritis; | Inspirion Delivery Technologies; Hydromorphone, |
| | | | | | | Pain, musculoskeletal, general; Pain, | Napp; Ibuprofen (CR) + codeine, Napp; Ibuprofen + |
| | | | | | | neuropathic; Pain, post-herpetic; Pain, post- | hydrocodone, Abbott; Ibuprofen combination, Adcock; |
| | | | | | | operative; Poisoning, drug; Premature | Levorphanol, TheraQuest; Methylnaltrexone, |
| | | | | | | ejaculation; Pruritus; Restless legs syndrome; | Progenics; Mirfentanil; Morphine + naltrexone, |
| | | | | | | Rhinitis, allergic, general; Rhinitis, general; | Alpharma; Morphine + oxycodone, QRx (oral); |
| | | | | | | Sepsis; Urinary retention | Morphine + oxycodone, QRx-2; Morphine + |
| | | | | | | | oxycodone, QRx (iv); Morphine SR, Takeda; Morphine |
| | | | | | | | sulfate, aaiPharma; Morphine, DepoFoam; Morphine, |
| | | | | | | | Egalet; Morphine, Elan; Morphine, Ethypharm; |
| | | | | | | | Morphine, Hospira; Morphine, Inspirion Delivery |
| | | | | | | | Technologies; Morphine, Javelin; Morphine, Karo Bio; |
| | | | | | | | Morphine, Nycomed Amersham; Morphine, Paion; |
| | | | | | | | Morphine, Rhotard; Morphine, Takeda; Morphine, |
| | | | | | | | Wyeth; Morphine-6-glucuronide, Paion; Mu opioid, iv, |
| | | | | | | | Trevena; Mu opioid, oral, Trevena; Nalbuphine; |
| | | | | | | | Nalbuphine, nasal; Nalmefene; Nalmefene, BioTie; |
| | | | | | | | Naloxol, Nektar; Naloxone, Bristol-Myers Squibb; |
| | | | | | | | Naloxone, Cosmo; Naloxone, Lightlake Therapeutics; |
| | | | | | | | Naloxone, SLA Pharma; Naloxone, lotion, Elorac; |
| | | | | | | | Naloxone, sublingual film, Aoxing; Naltrexone; |
| | | | | | | | Naltrexone, Alkermes; Naltrexone, Pain Ther; |
| | | | | | | | Oxycodone (iv), Napp; Oxycodone + acetaminophen + |
| | | | | | | | niacin, Acura; |
| OPRM1 | opioid receptor, | Coronary | 78 | rs675026-? | Coronary heart | Addiction, alcohol; Addiction, cocaine; | Oxycodone + acetaminophen, Labopharm; Oxycodone + |
| continued | mu 1 | heart disease | | (0.72) | disease | Addiction, drug, unspecified; Addiction, | ibuprofen, BTG; Oxycodone + naloxone, Purdue; |
| | | | | | | gambling; Addiction, narcotic/opiate; | Oxycodone + naltrexone, Elite; Oxycodone + |
| | | | | | | Addiction, nicotine; Anaesthesia; Anaesthesia, | naltrexone, Pain T; Oxycodone + niacin, Acura; |
| | | | | | | adjunct; Binge eating disorder; Bulimia; Cancer, | Oxycodone + paracetamol, Covid; Oxycodone |
| | | | | | | lung, non-small cell; Constipation; Depression, | hydrochloride, Xano; Oxycodone patch, |
| | | | | | | general; Depression, major depressive | Phosphagenics; Oxycodone + dextromethorpan, |
| | | | | | | disorder; Dyskinesia, levodopa-induced; Ileus; | Oxycodone + naltrexone, Alpharma; Oxycodone, Acura; |
| | | | | | | Infarction, myocardial; Irritable bowel | Oxycodone, CR, Egalet; Oxycodone, Collegium; |
| | | | | | | syndrome; Migraine; Motility dysfunction, GI, | Oxycodone, Elite, CR; Oxycodone, |
| | | | | | | general; Nausea and vomiting, opiate-induced; | IntelliPharmaCeutics; Oxycodone, Purdue Pharma; |
| | | | | | | Neuropathy, diabetic; Obesity; Pain, cancer; | Oxycodone, extended-release, Inspirion Delivery |
| | | | | | | Pain, general; Pain, musculoskeletal, arthritis; | Technologies; Oxycodone, long-acting, Pain T; |
| | | | | | | Pain, musculoskeletal, general; Pain, | Oxymorphone ER, crush-resistant; Oxymorphone, |
| | | | | | | neuropathic; Pain, post-herpetic; Pain, post- | Endo-2; Oxymorphone, Inspirion Delivery |
| | | | | | | operative; Poisoning, drug; Premature | Technologies; Oxymorphone, Pain Therapeutics; |
| | | | | | | ejaculation; Pruritus; Restless legs syndrome; | Oxymorphone, TIMERx; Paracetamol + codeine, Alza; |
| | | | | | | Rhinitis, allergic, general; Rhinitis, general; | Paracetamol + dihydrocodeine, Na; Remifentanil; |
| | | | | | | Sepsis; Urinary retention | Remifentanil, Vyteris; Sameridine; Sufentanil; |
| | | | | | | | Sufentanil + triazolam, AcelRx; Sufentanil, Labtec; |
| | | | | | | | Sufentanil, TRANSDUR; Sufentanil, sublingual, AcelRx; |
| | | | | | | | Sufentanil, sublingual, AcelRx-2; Sufentanil, sublingual, |
| | | | | | | | AcelRx-3; Tapentadol, ER; Tapentadol, IR; Tilidine + |
| | | | | | | | naloxone, Aoxing Pharmaceutical; Tilidine |
| | | | | | | | hydrochloride, Aoxing Pharmaceutical; Tramadol; |
| | | | | | | | Tramadol + NSAID, Biovai; Tramadol hydrochloride + |
| | | | | | | | erectile dysfunction therapy; Tramadol hydrochloride, |
| | | | | | | | Ampio Pharmaceuticals; Tramadol, Ciph |
| OSM | oncostatin M | Nephropathy | 23 | rs2412971-? | Nephropathy | Arthritis, rheumatoid | 3152 |
| | | | | (0.49 (EA)) | | | |
| PCSK9 | proprotein | Coronary | 78 | rs11206510-T | Coronary heart | Atherosclerosis; Hypercholesterolaemia; | ALN-PCS; AMG-145; BMS-844421; BMS-PCSK9Rx-2; |
| | convertase | heart disease | | (0.82) | disease | Hyperlipidaemia, general | LDL-4X4; PCSK9 Adnectin, Bristol-Myers Squibb; PCSK9 |
| | subtilisin/kexin | | | | | | siRNA drug signal, Nativis; REGN-727; REGN-728; RN- |
| | type 9 | | | | | | 316; SPC-50 |
| PDE10A | phosphodiesterase | Conduct | 17 | rs7762160-? | Conduct | Addiction, narcotic/opiate; Alzheimer's | 8045; EVP-6308; EVX-001650; PDE 10 inhibitors, |
| | 10A | disorder | | (0.365) | disorder | disease; Asthma; Conjunctivitis, inflammatory; | Omeros; PDE-10 inhibitor, Biocrea; PDE-2/10 inhibitor, |
| | | | | | | Infarction, cerebral; Insulin-related metabolic | Biocrea; PDE-Xa inhibitors, Evotec; PF-2545920; |
| | | | | | | syndrome; Multiple sclerosis, general; | Ibudilast; Phosphodiesterase X inhibitor, Lundbe |
| | | | | | | Neuropathy, diabetic; Pain, chemotherapy- | |
| | | | | | | induced; Pain, neuropathic; Psychosis, bipolar; | |
| | | | | | | Psychosis, general; Schizophrenia | |
| PDE2A | phosphodiesterase | Optic disc size | 17 | rs12418204-G | Optic disc size | Adenoma, colorectal; Alzheimer's disease; | CEL-031; PDE-2/10 inhibitor, Biocrea; Exisuli |
| | 2A, cGMP- | | | (NR) | | Barrett's oesophagus; Cancer, bladder; Cancer, | |
| | stimulated | | | | | breast; Cancer, leukaemia, chronic | |
| | | | | | | lymphocytic; Cancer, liver; Cancer, lung, non- | |
| | | | | | | small cell; Cancer, lung, small cell; Cancer, | |
| | | | | | | prostate; Cancer, renal; Crohn's disease; | |
| | | | | | | Inflammatory bowel disease, general | |
| PDE3A | phosphodiesterase | Male | 5 | rs10841496-? | Male infertility | Asthma; Atherosclerosis; Buerger's syndrome; | CR-3465; RPL-554; Amrinone; Anagrelide |
| | 3A, cGMP- | infertility | | (0.58) | | Chronic obstructive pulmonary disease; | hydrochloride; Cilostazol; Enoximone; Loprinone |
| | inhibited | | | | | Diabetic complication, general; Heart failure; | hydrochloride; Milrinone; Oberadilol; Parogrelil |
| | | | | | | Infarction, cerebral; Inflammation, urinary | hydrochloride; Pumafentrine; Tipeluka |
| | | | | | | tract; Peripheral vascular disease; | |
| | | | | | | Polycythaemia vera; Rhinitis, allergic, seasonal; | |
| | | | | | | Thrombocythaemia; Thrombocytosis | |
| PDE4D | phosphodiesterase | Esophageal | 15 | rs10052657-C | Esophageal | Addiction, narcotic/opiate; Alzheimer's | 8045; AN-2728; AN-2898; ASP-9831; CC-10015; CC- |
| | 4D, cAMP- | cancer | | (0.78) | cancer | disease; Ankylosing spondylitis; Anxiety, | 11050; COPD therapy, Spring Bank; D-22888; DE-103; |
| | specific | | | | | general; Arthritis, general; Arthritis, psoriatic; | DG-07.1; DWP-205297; GSK-256066; GSK-356278; GW- |
| | | | | | | Arthritis, rheumatoid; Asthma; Behcet's | 3600; IPL-455903; KW-4490; LAS-37779; OX-914; RPL- |
| | | | | | | disease; Chronic obstructive pulmonary | 554; RPR-122818 derivatives; RPR-132294; TA-7906; |
| | | | | | | disease; Cognitive disorder, unspecified; Colitis, | TPI-1100; V-11294A; Apremilast; Filaminast; Fosfosal; |
| | | | | | | ulcerative; Conjunctivitis, inflammatory; | Ibudilast; Loprinone hydrochloride; Mesopram; |
| | | | | | | Crohn's disease; Depression, major depressive | Oglemilast; Paclitaxel + triflusal stent; Pumafentrine; |
| | | | | | | disorder; Eczema, atopic; Eczema, contact; | Revamilast; Roflumilast; Ronomilast; Simvastatin + |
| | | | | | | Heart failure; Infarction, cerebral; Infarction, | triflusal stent; Tetomilast; Tipelukast; Triflus |
| | | | | | | myocardial; Inflammation, general; | |
| | | | | | | Inflammation, urinary tract; Inflammatory | |
| | | | | | | bowel disease, general; Ischaemia, cerebral; | |
| | | | | | | Lupus erythematosus, cutaneous; Multiple | |
| | | | | | | sclerosis, general; Multiple sclerosis, relapsing- | |
| | | | | | | remitting; Neuropathy, diabetic; Pain, | |
| | | | | | | chemotherapy-induced; Pain, general; Pain, | |
| | | | | | | musculoskeletal, arthritis; Pain, neuropathic; | |
| | | | | | | Pruritus; Psoriasis; Reperfusion injury; | |
| | | | | | | Respiratory disease, general; Rhinitis, allergic, | |
| | | | | | | general; Rhinitis, allergic, seasonal; Sarcoidosis; | |
| | | | | | | Steatohepatitis; Thrombosis, general | |
| PDX1 | pancreatic and | Attention | 30 | rs9512900-C | Attention deficit | Cancer, neuroendocrine, pancreatic; Cancer, | Pbi-shPDX-1 LP, Gradal |
| | duodenal | deficit | | (0.37) | hyperactivity | pancreatic | |
| | homeobox 1 | hyperactivity | | | disorder and | | |
| | | disorder and | | | conduct | | |
| | | conduct | | | disorder | | |
| | | disorder | | | | | |
| PIM3 | pim-3 oncogene | Ulcerative | 96 | rs5771069-G | Ulcerative colitis | Cancer, leukaemia, acute lymphocytic; Cancer, | CX-6258; SGI-1776; Pim kinase inhibitors, Jas |
| | | colitis | | (0.51) | | leukaemia, acute myelogenous; Cancer, | |
| | | | | | | leukaemia, general; Cancer, lymphoma, non- | |
| | | | | | | Hodgkin's; Cancer, pancreatic; Cancer, prostate | |
| PLA2G4A | phospholipase | Knee | 6 | rs4140564-? | Knee | Arthritis, rheumatoid; Glomerulonephritis; | AVX-001; AVX-002; AVX-0 |
| | A2, group IVA | osteoarthritis | | (0.05) | osteoarthritis | Psoriasis | |
| | (cytosolic, | | | | | | |
| | calcium- | | | | | | |
| | dependent) | | | | | | |
| POMC | proopiomelanocortin | Body mass | 70 | rs713586-C | Body mass index | Poisoning, metal | Melanotropin alpha, Mondobiote |
| | | index | | (0.47) | | | |
| PPARD | peroxisome | Response to | 18 | rs9658108-? | Response to | Atherosclerosis; Diabetes, Type 2; | CER-002; DB-959; HPP-593; MBX-8025; NN-0606; |
| | proliferator- | antipsychotic | | (0.052) | antipsychotic | Hypercholesterolaemia; Hyperlipidaemia, | PPAR pan-agonists, Plexxikon-3; PPAR-alpha/delta |
| | activated | treatment | | | treatment | general; Hypertriglyceridaemia; Insulin-related | agonists, Genfit; PPAR-delta agonist, Nippon-2; |
| | receptor delta | | | | | metabolic syndrome; Multiple sclerosis, | Multiple sclerosis therapy, Plexxik |
| | | | | | | general; Obesity | |
| PRKCE | protein kinase C, | Hemoglobin | 7 | rs10495928-G | Hemoglobin | Arthritis, rheumatoid; Diabetes, Type 2; | CDE-6960; KAI-1678; PN-2034; Sotrastaur |
| | epsilon | | | (NR) | | Hyperuricaemia; Obesity; Pain, neuropathic; | |
| | | | | | | Pain, post-herpetic; Pain, post-operative; | |
| | | | | | | Psoriasis; Transplant rejection, general; Uveitis | |
| PRKCQ | protein kinase C, | Type 1 | 50 | rs11258747-? | Type 1 diabetes | Arthritis, rheumatoid; Multiple sclerosis, | CDE-6960; R-057; R-461; Sotrastaur |
| | theta | diabetes | | (NR), | | general; Psoriasis; Transplant rejection, bone | |
| | | | | rs947474-G | | marrow; Transplant rejection, general; Uveitis | |
| | | | | (0.19) | | | |
| PRNP | prion protein | Creutzfeldt- | 2 | rs1799990-A | Creutzfeldt- | Alzheimer's disease | Cognition enhancers, Axerion Therapeuti |
| | | Jakob disease | | (NR) | Jakob disease | | |
| PTGER4 | prostaglandin E | Ulcerative | 96 | rs6451493-T | Ulcerative colitis | Allergy, general; Arthritis, osteo; Bone | BGC20-1531; CJ-23423; CR-5790; MF-592; PGN-9863; |
| | receptor 4 | colitis | | (0.61) | | disorder, general; Inflammation, general; | RQ-7; RQ-8; Anti-inflammatory, Merck & |
| | (subtype EP4) | | | | | Migraine; Pain, general; Pain, musculoskeletal, | |
| | | | | | | arthritis; Pain, musculoskeletal, general | |
| PTGER4 | prostaglandin E | Multiple | 59 | rs6896969-C | Multiple | Allergy, general; Arthritis, osteo; Bone | BGC20-1531; CJ-23423; CR-5790; MF-592; PGN-9863; |
| | receptor 4 | sclerosis | | (0.62) | sclerosis | disorder, general; Inflammation, general; | RQ-7; RQ-8; Anti-inflammatory, Merck & |
| | (subtype EP4) | | | | | Migraine; Pain, general; Pain, musculoskeletal, | |
| | | | | | | arthritis; Pain, musculoskeletal, general | |
| PTK2 | PTK2 protein | Response to | 4 | rs4961252-G | Response to | Cancer, breast; Cancer, colorectal; Cancer, | CEP-37440; CFAK-C4; CFAK-Y15; GSK-2256098; PF- |
| | tyrosine kinase 2 | interferon | | (0.40) | interferon beta | gastrointestinal, stomach; Cancer, head and | 04554878; PF-5622 |
| | | beta therapy | | | therapy | neck; Cancer, lung, general; Cancer, lung, non- | |
| | | | | | | small cell; Cancer, lymphoma, non-Hodgkin's; | |
| | | | | | | Cancer, neuroendocrine, general; Cancer, | |
| | | | | | | neuroendocrine, neuroblastoma; Cancer, | |
| | | | | | | ovarian; Cancer, pancreatic; Cancer, prostate; | |
| | | | | | | Cancer, solid, general; Cancer, squamous cell | |
| PTPN11 | protein tyrosine | Type 1 | 50 | rs6679677-A | Type 1 diabetes | Cancer, colorectal; Cancer, head and neck; | AEG-19915; Sodium stibogluconate, VioQue |
| | phosphatase, | diabetes | | (0.10) | | Cancer, leukaemia, general; Cancer, | |
| | non-receptor | | | | | melanoma; Cancer, mesothelioma; Cancer, | |
| | type 11 | | | | | neuroendocrine, general; Cancer, ovarian; | |
| | | | | | | Cancer, pancreatic; Cancer, prostate; Cancer, | |
| | | | | | | renal; Cancer, solid, general; Cancer, squamous | |
| | | | | | | cell; Infection, leishmaniasis; Nerve injury, | |
| | | | | | | general; Neuropathy, diabetic | |
| PTPN11 | protein tyrosine | Retinal | 18 | rs10774625-A | Retinal vascular | Cancer, colorectal; Cancer, head and neck; | AEG-19915; Sodium stibogluconate, VioQue |
| | phosphatase, | vascular | | (0.48) | caliber | Cancer, leukaemia, general; Cancer, | |
| | non-receptor | caliber | | | | melanoma; Cancer, mesothelioma; Cancer, | |
| | type 11 | | | | | neuroendocrine, general; Cancer, ovarian; | |
| | | | | | | Cancer, pancreatic; Cancer, prostate; Cancer, | |
| | | | | | | renal; Cancer, solid, general; Cancer, squamous | |
| | | | | | | cell; Infection, leishmaniasis; Nerve injury, | |
| | | | | | | general; Neuropathy, diabetic | |
| RAF1 | v-raf-1 murine | Cardiac | 22 | rs3729931-? | Cardiac | Cancer, bladder; Cancer, brain; Cancer, breast; | BIIB-024; DP-2874; Debio-0928; ISIS-5132; LErafAON- |
| | leukemia viral | hypertrophy | | (NR) | hypertrophy | Cancer, colorectal; Cancer, gastrointestinal, | ETU; PLX-5568; Pharmaprojects No. 6222; RG-7304; |
| | oncogene | | | | | stomach; Cancer, general; Cancer, head and | RO-5126766; SAR-397769; STP-503; XL-281; ICo-007; |
| | homolog 1 | | | | | neck; Cancer, leukaemia, acute myelogenous; | Sorafen |
| | | | | | | Cancer, leukaemia, chronic myelogenous; | |
| | | | | | | Cancer, liver; Cancer, lung, non-small cell; | |
| | | | | | | Cancer, lung, small cell; Cancer, melanoma; | |
| | | | | | | Cancer, mesothelioma; Cancer, myeloma; | |
| | | | | | | Cancer, neuroendocrine, carcinoid; Cancer, | |
| | | | | | | ovarian; Cancer, pancreatic; Cancer, | |
| | | | | | | peritoneal; Cancer, prostate; Cancer, renal; | |
| | | | | | | Cancer, solid, general; Cancer, thyroid; | |
| | | | | | | Infection, hepatitis-C virus; Macular | |
| | | | | | | degeneration, age-related, wet; | |
| | | | | | | Myelodysplastic syndrome; Oedema, macular, | |
| | | | | | | diabetic; Pain, general; Pain, neuropathic; | |
| | | | | | | Polycystic kidney disease; Retinopathy, | |
| | | | | | | diabetic | |
| RARB | retinoic acid | Obesity | 29 | rs1435703-T | Obesity | Acne; Cancer, general; Cancer, leukaemia, | Epiduo; Adapalene; Tamibarotene; Tazarotene; |
| | receptor, beta | | | (0.06) | | acute myelogenous; Cancer, liver; Cancer, lung, | Tazarotene, or |
| | | | | | | non-small cell; Cancer, myeloma; Crohn's | |
| | | | | | | disease; Multiple sclerosis, general; Psoriasis; | |
| | | | | | | Wound healing | |
| RET | ret proto- | Hirschsprung's | 4 | rs2742234-T | Hirschsprung's | Cancer, biliary; Cancer, bladder; Cancer, brain; | DCC-2157; SAR-302503; Apatinib; Cabozantinib; |
| | oncogene | disease | | (NR) | disease | Cancer, breast; Cancer, colorectal; Cancer, | Motesanib diphosphate; Regorafenib; Sorafenib; |
| | | | | | | endometrial; Cancer, fallopian tube; Cancer, | Sunitinib malate; Vandetan |
| | | | | | | gastrointestinal, stomach; Cancer, | |
| | | | | | | gastrointestinal, stromal; Cancer, head and | |
| | | | | | | neck; Cancer, leukaemia, acute myelogenous; | |
| | | | | | | Cancer, leukaemia, chronic myelogenous; | |
| | | | | | | Cancer, liver; Cancer, lung, non-small cell; | |
| | | | | | | Cancer, lung, small cell; Cancer, lymphoma, T- | |
| | | | | | | cell; Cancer, lymphoma, general; Cancer, | |
| | | | | | | melanoma; Cancer, mesothelioma; Cancer, | |
| | | | | | | myeloma; Cancer, neuroendocrine, carcinoid; | |
| | | | | | | Cancer, neuroendocrine, general; Cancer, | |
| | | | | | | neuroendocrine, pancreatic; Cancer, | |
| | | | | | | oesophageal; Cancer, ovarian; Cancer, | |
| | | | | | | pancreatic; Cancer, peritoneal; Cancer, | |
| | | | | | | prostate; Cancer, renal; Cancer, sarcoma, | |
| | | | | | | general; Cancer, sarcoma, glial; Cancer, solid, | |
| | | | | | | general; Cancer, thyroid; Idiopathic | |
| | | | | | | myelofibrosis; Myelodysplastic syndrome; | |
| | | | | | | Polycythaemia vera; Thrombocytopenia, | |
| | | | | | | general | |
| ROBO1 | roundabout, axon | Brain imaging | 8 | rs9836484-? | Brain imaging in | Infection, HIV prophylaxis | FGI-1 |
| | guidance | in | | (0.32) | schizophrenia | | |
| | receptor, | schizophrenia | | | | | |
| | homolog 1 | | | | | | |
| | (Drosophila) | | | | | | |
| RORA | RAR-related | Asthma | 19 | rs11071559-C | Asthma | Cancer, leukaemia, acute lymphocytic | Anti-ROR-1 MAbs, Kancera; Anticancer therapy, Kance |
| | orphan receptor A | | | (0.86) | | | |
| RORA | RAR-related | Response to | 5 | rs809736-? | Response to | Cancer, leukaemia, acute lymphocytic | Anti-ROR-1 MAbs, Kancera; Anticancer therapy, Kance |
| | orphan receptor A | citalopram | | (0.17) | citalopram | | |
| | | treatment | | | treatment | | |
| RORA | RAR-related | Depression-- | 9 | rs12912233-T | Depression-- | Cancer, leukaemia, acute lymphocytic | Anti-ROR-1 MAbs, Kancera; Anticancer therapy, Kance |
| | orphan receptor A | quantitative | | (0.46) | quantitative | | |
| | | trait | | | trait | | |
| RTN4 | reticulon 4 | Obesity | 29 | rs6726292-G | Obesity | Amyotrophic lateral sclerosis; Multiple | ATI-355; GSK-1223249; Nogo receptor, Axerion |
| | | | | (0.73) | | sclerosis, general; Spinal cord injury | Therapeuti |
| RXRG | retinoid X | AIDS | 3 | rs10800098-A | AIDS | Cancer, breast; Cancer, head and neck; Cancer, | Bexarotene, gel, Ligand; Bexarotene, oral, Eis |
| | receptor, gamma | | | (0.05) | | leukaemia, acute myelogenous; Cancer, lung, | |
| | | | | | | non-small cell; Cancer, lymphoma, T-cell; | |
| | | | | | | Cancer, ovarian; Cancer, renal; Cancer, | |
| | | | | | | sarcoma, Kaposi's; Diabetes, Type 2; Psoriasis | |
| RYR2 | ryanodine | Acute | 13 | rs7554607-A | Acute | Arrhythmia, general; Heart failure | Rycal, cardiac, Arm |
| | receptor 2 | lymphoblastic | | (0.56) | lymphoblastic | | |
| | (cardiac) | leukemia | | | leukemia | | |
| RYR2 | ryanodine | Hypertension | 22 | rs2820037-T | Hypertension | Arrhythmia, general; Heart failure | Rycal, cardiac, Arm |
| | receptor 2 | | | (0.14) | | | |
| | (cardiac) | | | | | | |
| RYR2 | ryanodine | Response to | 1 | rs2819742-G | Response to | Arrhythmia, general; Heart failure | Rycal, cardiac, Arm |
| | receptor 2 | cerivastatin | | (0.62) | cerivastatin | | |
| | (cardiac) | | | | | | |
| SCARB1 | scavenger | Renal cell | 2 | rs4765623-? | Renal cell | Infection, hepatitis-C virus | ITX-45 |
| | receptor class B, | carcinoma | | (0.34) | carcinoma | | |
| | member 1 | | | | | | |
| SCN10A | sodium channel, | Atrioventricular | 6 | rs6800541-C | Atrioventricular | Inflammation, general; Pain, cancer; Pain, | A-887826; CR-4892; NW-3509; Nav1.8 blocker, Pfizer- |
| | voltage-gated, | conduction | | (0.41) | conduction | general; Pain, neuropathic; Schizophrenia | 1; PN3 ZFP TF; SCN9A channel modulators, Icagen; Z-2 |
| | type X, alpha | | | | | | |
| | subunit | | | | | | |
| SELL | selectin L | Amyotrophic | 25 | rs3177980-G | Amyotrophic | Acute lung injury; Anaemia, sickle cell; Asthma; | GMI-1070; Bimosiamo |
| | | lateral | | (NR) | lateral sclerosis | Chronic obstructive pulmonary disease; | |
| | | sclerosis | | | | Eczema, atopic; Epilepsy, general; Psoriasis; | |
| | | | | | | Respiratory distress syndrome, adult | |
| SELPLG | selectin P ligand | Conduct | 17 | rs8179116-? | Conduct | Psoriasis | AbGn-1 |
| | | disorder | | (0.021) | disorder | | |
| SLC12A2 | solute carrier | Brain imaging | 8 | rs245201-? | Brain imaging in | Hypertension, general; Ocular disorder, | Bumetanide; Ethacrynic acid, Telor; Furosemide |
| | family 12 | in | | (0.32) | schizophrenia | unspecified; Oedema, cardiac; Oedema, | AcuForm, Depomed; Piretanide; Torasemide; |
| | (sodium/potassium/ | schizophrenia | | | | general | Torasemide ER, Penwest; Torasemide PR, Ferr |
| | chloride | | | | | | |
| | transporters), | | | | | | |
| | member 2 | | | | | | |
| SLC12A2 | solute carrier | Ileal | 4 | rs10089-? | Ileal carcinoids | Hypertension, general; Ocular disorder, | Bumetanide; Ethacrynic acid, Telor; Furosemide |
| | family 12 | carcinoids | | (NR) | | unspecified; Oedema, cardiac; Oedema, | AcuForm, Depomed; Piretanide; Torasemide; |
| | (sodium/potassium/ | | | | | general | Torasemide ER, Penwest; Torasemide PR, Ferr |
| | chloride | | | | | | |
| | transporters), | | | | | | |
| | member 2 | | | | | | |
| SLC12A3 | solute carrier | HDL | 37 | rs1800775-A | HDL cholesterol | Hypertension, general | Acebutolol + HCTZ; Aliskiren + HCTZ, Novartis; |
| | family 12 | cholesterol | | (0.49) | | | Aliskiren + amlodipine + HCTZ, Novartis; Amlodipine + |
| | (sodium/chloride | | | | | | losartan + HCTZ, Merck; Amlodipine + losartan + |
| | transporters), | | | | | | hydrochlorothiazide, Hanmi; |
| | member 3 | | | | | | Amlodipine + HCTZ + valsartan, Nov; Atenolol + |
| | | | | | | | bendroflumethiazide; Atenolol + chlorthalidone; |
| | | | | | | | Azilsartan medoxomil + chlorthalidone; Benazepril + |
| | | | | | | | HCTZ; Bisoprolol fumarate + HCTZ; |
| | | | | | | | Bisoprolol + trichloromethiazide; Candesartan cilexetil + |
| | | | | | | | HCTZ; Delapril + indapamide, Chiesi; Enalapril maleate + |
| | | | | | | | HCTZ; Eprosartan mesylate + HCTZ; Indapamide; |
| | | | | | | | Indapamide, Douglas; Indapamide, SR, Servier; |
| | | | | | | | Irbesartan + HCTZ; Losartan + HCTZ, BMS; Moexipril + |
| | | | | | | | HCTZ; Moxonidine + HCTZ, Abbott; Nebivolol + |
| | | | | | | | indapamide, Zydus Cadila; Olmesartan |
| | | | | | | | medoxomil + HCTZ, San; Perindopril + indapamide, |
| | | | | | | | Serv; Telmisartan + HCTZ; Valsartan + HC |
| SLC6A1 | solute carrier | Conduct | 17 | rs9990174-T | Conduct | Anxiety, general; Epilepsy, general | Deramciclane fumarate; Tiagabi |
| | family 6 | disorder | | (0.33) | disorder | | |
| | (neurotransmitter | | | | | | |
| | transporter, | | | | | | |
| | GABA), member 1 | | | | | | |
| SNCA | synuclein, alpha | Parkinson's | 47 | rs11931074-? | Parkinson's | Parkinson's disease | PD-01, Affiris; Parkinson's therapy, FoldRx; ReS12-S; |
| | (non A4 | disease | | (NR), | disease | | Synucle |
| | component of | | | rs2736990-? | | | |
| | amyloid | | | (NR), | | | |
| | precursor) | | | rs2736990-C | | | |
| | | | | (0.51), | | | |
| | | | | rs356219-G | | | |
| | | | | (0.39), | | | |
| | | | | rs356220-A | | | |
| | | | | (0.36), | | | |
| | | | | rs356220-T | | | |
| | | | | (0.35), | | | |
| | | | | rs356220-T | | | |
| | | | | (0.36) | | | |
| SOD1 | superoxide | Amyotrophic | 25 | rs13048019-T | Amyotrophic | Alzheimer's disease; Amyotrophic lateral | ALS MAb, Amorfix; ALS RNAi therapy, RXi; ALS vaccine, |
| | dismutase 1, | lateral | | (0.17) | lateral sclerosis | sclerosis; Cardiomyopathy, ischaemic; | Amorfix; Alzheimer's Ab therapy, Amorfix; Alzheimer's |
| | soluble | sclerosis | | | | Peyronie's disease; Radio/chemotherapy- | vaccine, Amorfix; ISIS-333611; Superoxide dismutase, |
| | | | | | | induced eczema; Wound healing | Polymun; Superoxide dismutase, U |
| STAT3 | signal transducer | Crohn's | 83 | rs744166-A | Crohn's disease | Arthritis, rheumatoid; Cancer, breast; Cancer, | OPB-31121; OPB-51602; Bardoxolone methyl; |
| | and activator of | disease | | (0.57) | | leukaemia, acute lymphocytic; Cancer, | Brivudine, RESprote |
| | transcription 3 | | | | | leukaemia, acute myelogenous; Cancer, | |
| | (acute-phase | | | | | leukaemia, chronic myelogenous; Cancer, | |
| | response factor) | | | | | leukaemia, general; Cancer, lung, non-small | |
| | | | | | | cell; Cancer, lymphoma, general; Cancer, | |
| | | | | | | lymphoma, non-Hodgkin's; Cancer, melanoma; | |
| | | | | | | Cancer, myeloma; Cancer, ovarian; Cancer, | |
| | | | | | | pancreatic; Cancer, renal; Cancer, solid, | |
| | | | | | | general; Cancer, thyroid; Hepatitis, non- | |
| | | | | | | infectious; Myelodysplastic syndrome; | |
| | | | | | | Nephropathy, diabetic; Renal failure | |
| STAT3 | signal transducer | Multiple | 59 | rs744166-G | Multiple | Arthritis, rheumatoid; Cancer, breast; Cancer, | OPB-31121; OPB-51602; Bardoxolone methyl; |
| | and activator of | sclerosis | | (0.41) | sclerosis | leukaemia, acute lymphocytic; Cancer, | Brivudine, RESprote |
| | transcription 3 | | | | | leukaemia, acute myelogenous; Cancer, | |
| | (acute-phase | | | | | leukaemia, chronic myelogenous; Cancer, | |
| | response factor) | | | | | leukaemia, general; Cancer, lung, non-small | |
| | | | | | | cell; Cancer, lymphoma, general; Cancer, | |
| | | | | | | lymphoma, non-Hodgkin's; Cancer, melanoma; | |
| | | | | | | Cancer, myeloma; Cancer, ovarian; Cancer, | |
| | | | | | | pancreatic; Cancer, renal; Cancer, solid, | |
| | | | | | | general; Cancer, thyroid; Hepatitis, non- | |
| | | | | | | infectious; Myelodysplastic syndrome; | |
| | | | | | | Nephropathy, diabetic; Renal failure | |
| SUMF1 | sulfatase | Multiple | 5 | rs794185-? | Multiple | Mucopolysaccharidosis IIIA | Sulfamidase, Lysoge |
| | modifying factor 1 | sclerosis-- | | (NR) | sclerosis--Brain | | |
| | | Brain | | | Glutamate | | |
| | | Glutamate | | | Levels | | |
| | | Levels | | | | | |
| TAP1 | transporter 1, | Nephropathy | 23 | rs9357155-? | Nephropathy | Cancer, breast | Breast cancer vaccine, Taplmmu |
| | ATP-binding | | | (0.87 (EA)) | | | |
| | cassette, sub- | | | | | | |
| | family B | | | | | | |
| | (MDR/TAP) | | | | | | |
| TERT | telomerase | Idiopathic | 1 | rs2736100-A | Idiopathic | Anaemia, aplastic; Cancer, bladder; Cancer, | 51145; BSU-1021; CB-10-01; GV-1001; GX-301; |
| | reverse | pulmonary | | (0.41) | pulmonary | brain; Cancer, breast; Cancer, colorectal; | Pharmaprojects No. 5840; TAT-153; TeloB-VAX; |
| | transcriptase | fibrosis | | | fibrosis | Cancer, gastrointestinal, general; Cancer, | V934/V935; VX-001; Telomerase vaccine, Ger |
| | | | | | | general; Cancer, leukaemia, acute | |
| | | | | | | myelogenous; Cancer, liver; Cancer, lung, non- | |
| | | | | | | small cell; Cancer, melanoma; Cancer, | |
| | | | | | | pancreatic; Cancer, prostate; Cancer, renal; | |
| | | | | | | Cancer, solid, general; Cirrhosis, hepatic; | |
| | | | | | | Fibrosis, pulmonary; Myelodysplastic | |
| | | | | | | syndrome | |
| TH | tyrosine | Prostate | 30 | rs7127900-A | Prostate cancer | Parkinson's disease | ProSav |
| | hydroxylase | cancer | | (0.20) | | | |
| TLR7 | toll-like receptor 7 | Celiac disease | 65 | rs5979785-? | Celiac disease | Arthritis, rheumatoid; Asthma; Atherosclerosis; | 12717; ANA-773; ANA-975; AZD-8848; DV-1179; GS- |
| | | | | (0.74) | | Cancer, basal cell; Cancer, bladder; Cancer, | 9620; IMO-3100; IPH-3201; TMX-201; TMX-202; VTX- |
| | | | | | | melanoma; Cancer, skin, general; Cancer, solid, | 463; Resiquimod; Vaccine adjuvants, TLR-7/8/9, Ide |
| | | | | | | general; Infection, hepatitis-B virus; Infection, | |
| | | | | | | hepatitis-C virus; Infection, herpes simplex | |
| | | | | | | virus; Infection, human papilloma virus; Lupus | |
| | | | | | | erythematosus, general; Multiple sclerosis, | |
| | | | | | | general; Psoriasis; Rhinitis, allergic, general; | |
| | | | | | | Rhinitis, allergic, seasonal; Vaccine adjunct | |
| TLR8 | toll-like receptor 8 | Celiac disease | 65 | rs5979785-? | Celiac disease | Arthritis, rheumatoid; Atherosclerosis; Cancer, | IMO-3100; IPH-3201; VTX-2337; VTX-463; Resiquimod; |
| | | | | (0.74) | | lymphoma, B-cell; Cancer, lymphoma, general; | Vaccine adjuvants, TLR-7/8/9, Ide |
| | | | | | | Cancer, solid, general; Infection, hepatitis-C | |
| | | | | | | virus; Infection, herpes simplex virus; Lupus | |
| | | | | | | erythematosus, general; Multiple sclerosis, | |
| | | | | | | general; Psoriasis; Rhinitis, allergic, general; | |
| | | | | | | Vaccine adjunct | |
| TNF | tumor necrosis | Neonatal | 10 | rs3099844-? | Neonatal lupus | Alzheimer's disease; Amyloidosis; Anal fistula; | 61687; AG-014; ALKS-6931; AME-527; ART-621; AVX- |
| | factor | lupus | | (0.11) | | Ankylosing spondylitis; Arthritis, general; | 470; CEQ-600; CYT-020-TNFQb; ESBA-105; Humicade; |
| | | | | | | Arthritis, juvenile; Arthritis, osteo; Arthritis, | ISIS-104838; ISIS-104838, oral; P-979-03; PD-2015; PD- |
| | | | | | | psoriatic; Arthritis, rheumatoid; Asthma; | 2016; PD-2024; PF-05230905; Pharmaprojects No. |
| | | | | | | Behcet's disease; Cachexia; Cancer, biliary; | 5357; Pharmaprojects No. 5534; SSS-07; Sphira; |
| | | | | | | Cancer, brain; Cancer, breast; Cancer, | TNFQb; UR-1505; VGX-1027; VT-346; XPro-1595; YHB- |
| | | | | | | colorectal; Cancer, gastrointestinal, stomach; | 1411-2; Adalimumab; Anti-TNF Ab, Protherics; |
| | | | | | | Cancer, general; Cancer, head and neck; | Antidiabetic/antiobesity therapy, Avaxia; Arthritis |
| | | | | | | Cancer, lung, general; Cancer, lung, non-small | therapy, NanoSmart-1; Certolizumab pegol; |
| | | | | | | cell; Cancer, melanoma; Cancer, myeloma; | Etanercept; Etanercept, BioAssets Development; |
| | | | | | | Cancer, oesophageal; Cancer, ovarian; Cancer, | Etanercept, Cipla; Etanercept, Hanwha; Etanercept, LG |
| | | | | | | pancreatic; Cancer, prostate; Cancer, renal; | Life Sciences; Etanercept, Lafrancol S.A.; Etanercept, |
| | | | | | | Cancer, sarcoma, Kaposi's; Cancer, sarcoma, | Neurokine; Etanercept, Neurokine, liposomal; |
| | | | | | | soft tissue; Cancer, solid, general; Cancer, | Etanercept, Protalix; Etanercept, Zenotech; Fibromun; |
| | | | | | | thymoma; Cancer, thyroid; Cataract; Chronic | Glycophosphopeptical, Cantabria; Golimumab; |
| | | | | | | obstructive pulmonary disease; Coeliac | Golimumab, BioXpress; Golnerminogene pradenovec; |
| | | | | | | disease; Cognitive disorder, unspecified; Colitis, | Infliximab; Infliximab, BioXpress; Infliximab, Celltrion; |
| | | | | | | general; Colitis, ulcerative; Coronary artery | Nerelimomab; Oral mucositis therapy, Avaxia; |
| | | | | | | bypass grafting; Crohn's disease; Diabetes, | Ozoralizumab; Revamilast; TgAAC94; Thalidomide, |
| | | | | | | Type 1; Diabetes, Type 2; Diabetes, general; | Celge |
| | | | | | | Eczema, atopic; Fibrosis, pulmonary, | |
| | | | | | | idiopathic; Heart failure; Hidradenitis | |
| | | | | | | suppurativa; Infarction, myocardial; Infection, | |
| | | | | | | HIV/AIDS; Infection, hepatitis-C virus; Infection, | |
| | | | | | | leprosy; Infection, malaria; Infection, | |
| | | | | | | unspecified; Inflammation, general; | |
| | | | | | | Inflammation, ocular; Inflammatory bowel | |
| | | | | | | disease, general; Ischaemia, cerebral; Lupus | |
| | | | | | | erythematosus, cutaneous; Macular | |
| | | | | | | degeneration, age-related, general; Multiple | |
| | | | | | | sclerosis, general; Myelodysplastic syndrome; | |
| | | | | | | Obesity; Pain, complex regional; Pain, | |
| | | | | | | musculoskeletal, general; Pain, neuropathic; | |
| | | | | | | Psoriasis; Radio/chemotherapy-induced | |
| | | | | | | mucositis; | |
| TNF | tumor necrosis | Neonatal | 10 | rs3099844-? | Neonatal lupus | Respiratory distress syndrome, adult; | |
| continued | factor | lupus | | (0.11) | | Retinopathy, general; Sarcoidosis; Sepsis; | |
| | | | | | | Spondyloarthritis, axial; Transplant rejection, | |
| | | | | | | bone marrow; Transplant rejection, general; | |
| | | | | | | Ulcer, aphthous; Uveitis; Vaccine adjunct; | |
| | | | | | | Wegener's granulomatosis; Xerophthalmia | |
| TNFRSF1A | tumor necrosis | Primary | 26 | rs1800693-C | Primary biliary | Acute lung injury; Alzheimer's disease; | AKH-217; AP-301; ARRY-438162; CC-11050; CR-1; |
| | factor receptor | biliary | | (0.40) | cirrhosis | Ankylosing spondylitis; Arthritis, osteo; | ITMN-520; PMI-005; TNF-K; TNF-alpha, CytImmune; |
| | superfamily, | cirrhosis | | | | Arthritis, psoriatic; Arthritis, rheumatoid; | TNFR1 NAM, Addex; TT-301; VB-111; YP-008; Anti- |
| | member 1A | | | | | Asthma; Behcet's disease; Benign prostatic | TNFMAb, LG Life Sciences; Apremilast; Citoplurikin; |
| | | | | | | hyperplasia; Cancer, adrenal; Cancer, biliary; | Lenercept; Pirfenidone; Pirfenidone, GNI; Semapimodsa |
| | | | | | | Cancer, brain; Cancer, breast; Cancer, | |
| | | | | | | colorectal; Cancer, gastrointestinal, general; | |
| | | | | | | Cancer, head and neck; Cancer, liver; Cancer, | |
| | | | | | | lung, general; Cancer, lung, non-small cell; | |
| | | | | | | Cancer, melanoma; Cancer, ovarian; Cancer, | |
| | | | | | | pancreatic; Cancer, renal; Cancer, sarcoma, | |
| | | | | | | general; Cancer, sarcoma, glial; Cancer, | |
| | | | | | | sarcoma, soft tissue; Cancer, solid, general; | |
| | | | | | | Cancer, thyroid; Chronic obstructive pulmonary | |
| | | | | | | disease; Crohn's disease; Eczema, general; | |
| | | | | | | Fibrosis, general; Fibrosis, liver; Fibrosis, | |
| | | | | | | pulmonary; Fibrosis, pulmonary, idiopathic; | |
| | | | | | | Fibrosis, uterine; Haemorrhage, cerebral; Head | |
| | | | | | | trauma; Inflammation, general; Irritable bowel | |
| | | | | | | syndrome; Lupus erythematosus, cutaneous; | |
| | | | | | | Mucositis, general; Multiple sclerosis, general; | |
| | | | | | | Multiple sclerosis, progressive, general; | |
| | | | | | | Multiple sclerosis, progressive, secondary; | |
| | | | | | | Oedema, pulmonary; Pruritus; Psoriasis; | |
| | | | | | | Radio/chemotherapy-induced injury, general; | |
| | | | | | | Reperfusion injury; Respiratory distress | |
| | | | | | | syndrome, adult; Retinopathy, general; | |
| | | | | | | Sarcoidosis; Scleroderma; Sepsis; Wound | |
| | | | | | | healing | |
| TNFRSF21 | tumor necrosis | Panic disorder | 10 | rs2103868-? | Panic disorder | Multiple sclerosis, general | DR6 antagonist, Biogen Id |
| | factor receptor | | | (0.26) | | | |
| | superfamily, | | | | | | |
| | member 21 | | | | | | |
| TNFRSF9 | tumor necrosis | Celiac disease | 65 | rs12727642-A | Celiac disease | Cancer, lymphoma, B-cell; Cancer, lymphoma, | PF-050825 |
| | factor receptor | | | (0.19) | | non-Hodgkin's; Cancer, solid, general | |
| | superfamily, | | | | | | |
| | member 9 | | | | | | |
| TNFRSF9 | tumor necrosis | Ulcerative | 96 | rs35675666-G | Ulcerative colitis | Cancer, lymphoma, B-cell; Cancer, lymphoma, | PF-050825 |
| | factor receptor | colitis | | (0.83) | | non-Hodgkin's; Cancer, solid, general | |
| | superfamily, | | | | | | |
| | member 9 | | | | | | |
| TNFSF11 | tumor necrosis | Crohn's | 83 | rs2062305-G | Crohn's disease | Arthritis, rheumatoid; Bone disorder, general; | ALX-0141; CEP-37251; GST-RANKL, Auxeris; |
| | factor (ligand) | disease | | (0.35) | | Cancer, bone; Cancer, breast; Cancer, | Denosumab; Denosumab, BioXpre |
| | superfamily, | | | | | colorectal; Cancer, head and neck; Cancer, | |
| | member 11 | | | | | lung, non-small cell; Cancer, lymphoma, | |
| | | | | | | Hodgkin's; Cancer, lymphoma, general; Cancer, | |
| | | | | | | lymphoma, non-Hodgkin's; Cancer, myeloma; | |
| | | | | | | Cancer, prostate; Cancer, renal; Cancer, | |
| | | | | | | thyroid; Hypercalcaemia of malignancy; | |
| | | | | | | Osteoporosis; Regeneration, bone; | |
| | | | | | | Regeneration, bone, fracture healing | |
| TNFSF15 | tumor necrosis | Crohn's | 83 | rs3810936-C | Crohn's disease | Asthma | Inhibic |
| | factor (ligand) | disease | | (0.68), | | | |
| | superfamily, | | | rs4263839-G | | | |
| | member 15 | | | (0.68) | | | |
| TNFSF15 | tumor necrosis | Ulcerative | 96 | rs4246905-C | Ulcerative colitis | Asthma | Inhibic |
| | factor (ligand) | colitis | | (0.71) | | | |
| | superfamily, | | | | | | |
| | member 15 | | | | | | |
| TNFSF15 | tumor necrosis | Inflammatory | 17 | rs6478109-? | Inflammatory | Asthma | Inhibic |
| | factor (ligand) | bowel disease | | (0.69) | bowel disease | | |
| | superfamily, | | | | | | |
| | member 15 | | | | | | |
| TNFSF15 | tumor necrosis | Leprosy | 4 | rs6478108-A | Leprosy | Asthma | Inhibic |
| | factor (ligand) | | | (0.46) | | | |
| | superfamily, | | | | | | |
| | member 15 | | | | | | |
| TNFSF4 | tumor necrosis | Celiac disease | 65 | rs859637-A | Celiac disease | Asthma; Rhinitis, allergic, general | Oxelumab (I) |
| | factor (ligand) | | | (0.49) | | | |
| | superfamily, | | | | | | |
| | member 4 | | | | | | |
| TNFSF4 | tumor necrosis | Diabetic | 34 | rs1342038-G | Diabetic | Asthma; Rhinitis, allergic, general | Oxelumab (I) |
| | factor (ligand) | retinopathy | | (0.64) | retinopathy | | |
| | superfamily, | | | | | | |
| | member 4 | | | | | | |
| TNFSF4 | tumor necrosis | Systemic | 42 | rs2205960-A | Systemic lupus | Asthma; Rhinitis, allergic, general | Oxelumab (I) |
| | factor (ligand) | lupus | | (0.27) | erythematosus | | |
| | superfamily, | erythematosus | | | | | |
| | member 4 | | | | | | |
| TRHR | thyrotropin- | Body mass | 1 | rs7832552-T | Body mass | Alzheimer's disease; Amnesia; Ataxia, | 54580; JTP-2942; TRH, Daiichi; TRH, Ferring; TRH, |
| | releasing | | | (0.32) | | spinocerebellar; Dementia, vascular; | Japan Tobacco; Montirelin hydrate; Posatirelin; |
| | hormone | | | | | Depression, general; Diagnosis, general; | Protirelin, Takeda; Taltirelin hydrate; Taltirelin hydrate O |
| | receptor | | | | | Dyskinesia, general; Epilepsy, general; Head | |
| | | | | | | trauma; Ischaemia, cerebral | |
| TRPM8 | transient | Migraine | 7 | rs10166942-T | Migraine | Cancer, breast; Cancer, colorectal; Cancer, | D-3263; TRPM8 antagonists, Johnson & Johnson; |
| | receptor | | | (0.81) | | lung, general; Cancer, prostate; Cancer, solid, | TRPM8, Hydra Bioscienc |
| | potential cation | | | | | general; Pain, general | |
| | channel, | | | | | | |
| | subfamily M, | | | | | | |
| | member 8 | | | | | | |
| TSHR | thyroid | Optic disc size | 17 | rs17111394-T | Optic disc size | Cancer, thyroid; Diagnosis, cancer; Goitre, non- | Thyrotropin alfa, Sano |
| | stimulating | | | (NR) | | toxic | |
| | hormone | | | | | | |
| | receptor | | | | | | |
| TYK2 | tyrosine kinase 2 | Type 1 | 50 | rs2304256-C | Type 1 diabetes | Arthritis, rheumatoid; Cancer, haematological, | SGI-1252; Rheumatoid arthritis therapy, SRI |
| | | diabetes | | (0.71) | | general; Cancer, solid, general | Internation |
| TYK2 | tyrosine kinase 2 | Crohn's | 83 | rs12720356-G | Crohn's disease | Arthritis, rheumatoid; Cancer, haematological, | SGI-1252; Rheumatoid arthritis therapy, SRI |
| | | disease | | (0.08) | | general; Cancer, solid, general | Internation |
| TYK2 | tyrosine kinase 2 | Psoriasis | 30 | rs12720356-A | Psoriasis | Arthritis, rheumatoid; Cancer, haematological, | SGI-1252; Rheumatoid arthritis therapy, SRI |
| | | | | (0.90), | | general; Cancer, solid, general | Internation |
| | | | | rs280519-A | | | |
| | | | | (0.47) | | | |
| TYR | tyrosinase | Melanoma | 4 | rs1393350-A | Melanoma | Cancer, melanoma; Skin disorder, general | MKC-1106-MT; Kojic acid, Vinas; Melanoma vaccine, |
| | (oculocutaneous | | | (0.27) | | | Bavarian; Melanoma vaccine, Ich |
| | albinism IA) | | | | | | |
| TYR | tyrosinase | Vitiligo | 25 | rs1393350-G | Vitiligo | Cancer, melanoma; Skin disorder, general | MKC-1106-MT; Kojic acid, Vinas; Melanoma vaccine, |
| | (oculocutaneous | | | (0.733) | | | Bavarian; Melanoma vaccine, Ich |
| | albinism IA) | | | | | | |
| VEGFA | vascular | Chronic | 67 | rs881858-G | Chronic kidney | Amyotrophic lateral sclerosis; Arthritis, | AG-13958; ALN-VSP; ALS gene therapy, Oxford BioM; |
| | endothelial | kidney | | (0.28) | disease | rheumatoid; Atherosclerosis; Cancer, basal cell; | BFH-772; CEQ-300; CVX-241; IXSVEGF; KH-902; MP- |
| | growth factor A | disease | | | | Cancer, bladder; Cancer, brain; Cancer, breast; | 0112; MultiGeneAngio; NT-502; NT-503; PAN-90806; |
| | | | | | | Cancer, colorectal; Cancer, endometrial; | PRS-050; PTC-299; SB-509; STP-601; STP-801; VEGF |
| | | | | | | Cancer, fallopian tube; Cancer, gastrointestinal, | gene therapy, Human Stem Cells Institute; VEGF gene |
| | | | | | | stomach; Cancer, gastrointestinal, stromal; | therapy, Merck; VEGF inhibitors, Santen; VEGF |
| | | | | | | Cancer, general; Cancer, head and neck; | peptide, Johnson & Johnson; VEGF-2 gene therapy, |
| | | | | | | Cancer, leukaemia, acute myelogenous; | VIA; X-82; Aflibercept; Aflibercept ophthalmic |
| | | | | | | Cancer, leukaemia, chronic lymphocytic; | solution; Bevacizumab; Bevacizumab, BioXpress; |
| | | | | | | Cancer, leukaemia, mast cell; Cancer, liver; | Bevasiranib sodium; Lycium anti-angiogenic |
| | | | | | | Cancer, lung, non-small cell; Cancer, lung, small | proteoglycan, Retina Pharma; Midostaurin; |
| | | | | | | cell; Cancer, lymphoma, B-cell; Cancer, | Pegaptanib octasodium; Pegaptanib sodium, extended |
| | | | | | | lymphoma, non-Hodgkin's; Cancer, melanoma; | release, Eyetech; Ranibizumab; SFLT-01 gene therapy, |
| | | | | | | Cancer, mesothelioma; Cancer, myeloma; | AG |
| | | | | | | Cancer, neuroendocrine, carcinoid; Cancer, | |
| | | | | | | neuroendocrine, pancreatic; Cancer, ovarian; | |
| | | | | | | Cancer, pancreatic; Cancer, peritoneal; Cancer, | |
| | | | | | | prostate; Cancer, renal; Cancer, sarcoma, | |
| | | | | | | Kaposi's; Cancer, sarcoma, general; Cancer, | |
| | | | | | | sarcoma, soft tissue; Cancer, solid, general; | |
| | | | | | | Cancer, thyroid; Cardiomyopathy, ischaemic; | |
| | | | | | | Dysplasia, general; Glaucoma; Head trauma; | |
| | | | | | | Herpetic keratitis; Macular degeneration, age- | |
| | | | | | | related, general; Macular degeneration, age- | |
| | | | | | | related, wet; Malignant ascites; Mastocytosis; | |
| | | | | | | Myelodysplastic syndrome; Myopia; | |
| | | | | | | Nephropathy, diabetic; Neurofibromatosis; | |
| | | | | | | Neuropathy, diabetic; Neuropathy, | |
| | | | | | | unspecified; Oedema, macular; Oedema, | |
| | | | | | | macular, diabetic; Peripheral vascular disease; | |
| | | | | | | Psoriasis; Retinal vein occlusion; Retinopathy, | |
| | | | | | | diabetic; Spinal cord injury | |
| VEGFA | vascular | Type 2 | 61 | rs9472138-T | Type 2 diabetes | Amyotrophic lateral sclerosis; Arthritis, | AG-13958; ALN-VSP; ALS gene therapy, Oxford BioM; |
| | endothelial | diabetes | | (0.28) | | rheumatoid; Atherosclerosis; Cancer, basal cell; | BFH-772; CEQ-300; CVX-241; IXSVEGF; KH-902; MP- |
| | growth factor A | | | | | Cancer, bladder; Cancer, brain; Cancer, breast; | 0112; MultiGeneAngio; NT-502; NT-503; PAN-90806; |
| | | | | | | Cancer, colorectal; Cancer, endometrial; | PRS-050; PTC-299; SB-509; STP-601; STP-801; VEGF |
| | | | | | | Cancer, fallopian tube; Cancer, gastrointestinal, | gene therapy, Human Stem Cells Institute; VEGF gene |
| | | | | | | stomach; Cancer, gastrointestinal, stromal; | therapy, Merck; VEGF inhibitors, Santen; VEGF |
| | | | | | | Cancer, general; Cancer, head and neck; | peptide, Johnson & Johnson; VEGF-2 gene therapy, |
| | | | | | | Cancer, leukaemia, acute myelogenous; | VIA; X-82; Aflibercept; Aflibercept ophthalmic |
| | | | | | | Cancer, leukaemia, chronic lymphocytic; | solution; Bevacizumab; Bevacizumab, BioXpress; |
| | | | | | | Cancer, leukaemia, mast cell; Cancer, liver; | Bevasiranib sodium; Lycium anti-angiogenic |
| | | | | | | Cancer, lung, non-small cell; Cancer, lung, small | proteoglycan, Retina Pharma; Midostaurin; |
| | | | | | | cell; Cancer, lymphoma, B-cell; Cancer, | Pegaptanib octasodium; Pegaptanib sodium, extended |
| | | | | | | lymphoma, non-Hodgkin's; Cancer, melanoma; | release, Eyetech; Ranibizumab; SFLT-01 gene therapy, |
| | | | | | | Cancer, mesothelioma; Cancer, myeloma; | AG |
| | | | | | | Cancer, neuroendocrine, carcinoid; Cancer, | |
| | | | | | | neuroendocrine, pancreatic; Cancer, ovarian; | |
| | | | | | | Cancer, pancreatic; Cancer, peritoneal; Cancer, | |
| | | | | | | prostate; Cancer, renal; Cancer, sarcoma, | |
| | | | | | | Kaposi's; Cancer, sarcoma, general; Cancer, | |
| | | | | | | sarcoma, soft tissue; Cancer, solid, general; | |
| | | | | | | Cancer, thyroid; Cardiomyopathy, ischaemic; | |
| | | | | | | Dysplasia, general; Glaucoma; Head trauma; | |
| | | | | | | Herpetic keratitis; Macular degeneration, age- | |
| | | | | | | related, general; Macular degeneration, age- | |
| | | | | | | related, wet; Malignant ascites; Mastocytosis; | |
| | | | | | | Myelodysplastic syndrome; Myopia; | |
| | | | | | | Nephropathy, diabetic; Neurofibromatosis; | |
| | | | | | | Neuropathy, diabetic; Neuropathy, | |
| | | | | | | unspecified; Oedema, macular; Oedema, | |
| | | | | | | macular, diabetic; Peripheral vascular disease; | |
| | | | | | | Psoriasis; Retinal vein occlusion; Retinopathy, | |
| | | | | | | diabetic; Spinal cord injury | |
| VKORC1 | vitamin K epoxide | Warfarin | 4 | rs10871454-? | Warfarin | Ischaemia, cerebral; Thrombosis, general; | Tecarfar |
| | reductase | maintenance | | (NR), | maintenance | Thrombosis, venous | |
| | complex, subunit 1 | dose | | rs9923231-? | dose | | |
| | | | | (NR), | | | |
| | | | | rs9923231-T | | | |
| | | | | (0.40) | | | |
| YWHAG | tyrosine 3- | Multiple | 59 | rs17149161-A | Multiple | Fibrosis, pulmonary, idiopathic; Wound healing | AZX-1 |
| | monooxygenase/ | sclerosis | | (0.2), | sclerosis | | |
| | tryptophan 5- | | | rs7779014-T | | | |
| | monooxygenase | | | (0.2) | | | |
| | activation | | | | | | |
| | protein, gamma | | | | | | |
| | polypeptide | | | | | | |

The invention is further described by the following non-limiting examples.

### EXAMPLES

### Example 1

### Drug Repositioning as a Proposed Short-Term Clinical Benefit from Genome-Wide Association Studies

It is widely recognized that Genome Wide Association Studies (GWAS) have not met their broad promise to improve human health, yet an original specific GWAS aim was to identify new drug targets. In an attempt to evaluate the utility of GWAS for new drug targets, we investigated whether GWAS studies could point to unsuspected indications for existing drugs or drugs in development. Our analyses were based on all available data in the NHGRI GWAS catalogue as of February, 2011, comprising 4818 genetic associations in 796 publications, of which 1,515 are replicated associations (p value>1E-7) with non-anthropomorphic traits. 991 genes with HUGO names were identified from these replicated associations. A total of 212 (21%) and 469 (47%) of these genes encode proteins respectively considered as “druggable” by small molecules or biopharmaceuticals; i.e., GWAS-derived genes are significantly more likely than chance to be theoretically tractable drug targets. Of these, 155 genes (16%) are active targets for drug discovery or development programs in the pharmaceutical industry, a proportion which is 2.5 times the genome at large (6%); i.e., GWAS-identified genes are also practically more likely than chance to be drug targets. Many of the indications in these active drug targets match or are closely related to the GWAS disease trait (63/155), an emblematic example of which is 3-hydroxy-3-methylglutaryl-CoA reductase (HMGCR), encoding the target for lipid lowering statins, and which is strongly associated with LDL-cholesterol levels by GWAS. Potential opportunities for drug repositioning are even greater, with 92/155 genes revealing a mismatch between the GWAS trait and the drug indication. Examples include the necrosis factor (ligand) superfamily, member 11 (TNFS11), which encodes the target of the osteoporosis drug denosumab, and was genetically associated with Crohn's disease, and also interleukin 12A (IL12A), which encodes the target of the cancer drug briakinumab and is associated with primary biliary cirrhosis.

Considered together, these analyses suggest new translational applications for GWAS-identified genes as both theoretical and practical drug targets.

### Introduction

Over the last few years large investments have been made in genome-wide association studies (GWAS) with the expectations, among others, that these studies would lead to the identification of novel therapeutic modalities or allow selection of patients who would respond better to therapeutic interventions (The Wellcome Trust Case Control Consortium. Nature 447, 661-78 (2007)). Although the results have provided valuable biological insights for many common diseases (Hampe et al. Nat. Genet. 39, 207-211 (2007) and Zeggini et al. Nat. Genet. 40, 638-645 (2008)), the translation of the genetics findings from GWAS into the clinic remains limited and a topic of intense debate (Goldstein, D. B. N. Engl. J. Med. 360, 1696-1698 (2009) and Hirschorn, J. N. N. Engl. J. Med. 360, 1699-1701 (2009)). Some factors could explain this situation. First, the road from a gene target to an approved marketed drug takes in general more than 10 years and most GWAS results have only been obtained over the past 4 years. Second, because the effect size of the common variants identified by GWAS, alone or in aggregation, is generally modest, the impact in terms of personalized, individually tailored medicine has been negligible at this stage. Recently some general principles have been proposed for post-GWAS functional analysis of risk loci that could ultimately translate into clinical benefits (Freedman, M. L. et al. Nat. Genet. 43, 513-518 (2011)). Here we propose a faster route based on an original and fundamental GWAS aim of identifying new drug targets: by investigating if GWAS studies can be used as a pointer to alternative or refined indications for drugs in development or already on the market. We first analyzed if the genes in GWAS replicated′ loci are potentially amenable to pharmacological modulation. Next, we examined whether the GWAS genes are already targeted by marketed drugs or by those currently being developed by the pharmaceutical industry. We hypothesized that, when the disease indication of the drug matches the GWAS disease trait associated with the target gene, GWAS studies increase the confidence that the right indication is pursued and that, conversely, a mismatch would point to alternative indication for the drug, i.e. drug repositioning (Ashburn, T. T. and Thor, K. B. Nat. Rev. Drug. Discov. 3, 673-683 (2004)). This approach is particularly attractive because choosing the right indication is a major challenge in drug discovery and development, and is even more relevant for molecules already in phase II and beyond, for which a large body of data is usually available to show their safety profile.

### Results

### GWAS Associated Genes as Potential Pharmaceutical Targets

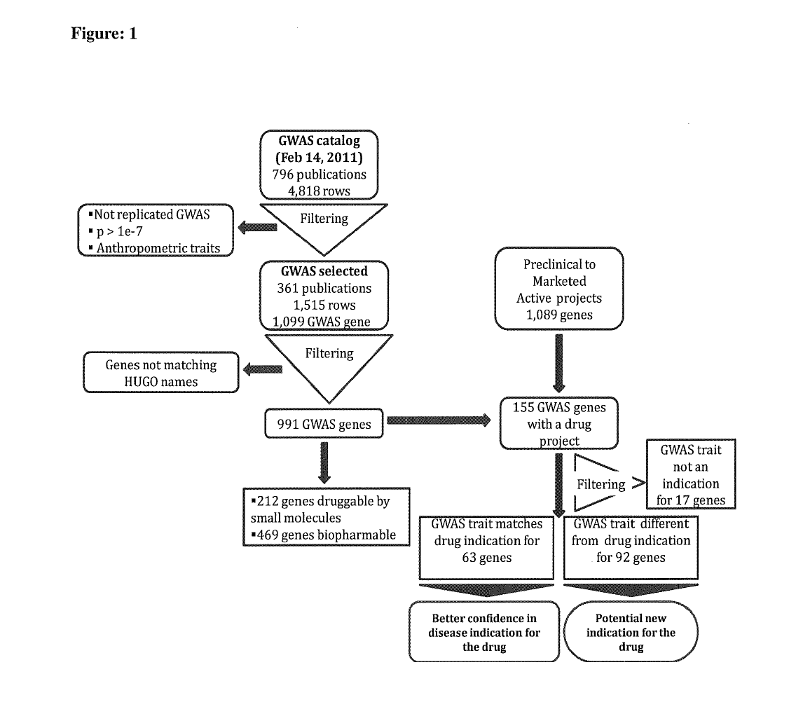

To construct a list of GWAS genes associated with disease traits we used the catalog of published GWAS studies from the National Human Genome Research Institute (NHGRI) (http://www.genome.gov/gwasstudies) (Hindorff, L. A. et al. Proc. Natl. Acad. Sci. USA 106, 9362-9367 (2009)). This resource contains an exhaustive description of trait/disease-associated Single Nucleotide Polymorphisms (SNP). At the time of our analysis (Feb. 14, 2011) the GWAS catalog contained 796 publications with 4,818 rows of data, each row corresponding to an association between a trait and an index SNP. The analysis workflow we used is described in FIG. 1 (see Methods for details). As a first step we eliminated 2,166 associations annotated as not replicated, and an additional 737 associations with p-value>1e-7, in an attempt to minimize the inclusion of false positive signals in our analysis. An additional 400 associations were excluded because the associated traits were anthropometric and not relevant in the drug discovery context of our analysis (see Table 1). The remaining 1,515 rows from 361 publications referred to 1,099 gene names. Of these genes, 991 were recognizable as approved HUGO gene names from Entrez Gene (Maglott, D. et al. Nucl. Acids Res. 39, D52-D57 (2011)). These 991 genes constituted the starting list for further analysis.

We next investigated how many of these 991 genes were amenable to pharmacological modulation using small molecules (in other words “druggable” (Hopkins, A. L. and Groom C. R. Nat. Rev. Drug Discov. 1, 727-730 (2002))), or biopharmaceuticals (in other words “biopharmable” using therapeutic antibodies or protein therapeutics), and compared these results with the entire genome. Out of 991 genes, 212 (21%) were considered “druggable” by small molecules, and 469 (47%) potentially “biopharmable”, defined here as being annotated with either a signal peptide or a transmembrane domain in ENSEMBL. These proportions are higher than those derived from the entire genome (corresponding to 19,258 genes with HUGO names (Seal, R. L. et al. Nucl. Acids Res. 39, D514-D519 (2011)) derived from the ENSEMBL database (Flicek, P. et al. Nucl. Acids Res. 39, D800-D806 (2011)), which contains 3,191 potentially “druggable” genes (17%, with p<5E-5) and 7,411 potentially “biopharmable” genes (38%, with p<6E-9) (FIG. 1).

Next we investigated if that excess in “druggable” or “biopharmable” genes among GWAS genes explained by differences in the proportion of housekeeping genes. We obtained a set of 2,375 housekeepers (Dezsö, Z. et al. BMC Biology 6, 49 (2008)) and observed that 105 of these genes overlapped with the GWAS set of 991 genes compared to an expected 122 (p<0.10). This is only slightly less than expected 122 (p<0.1). Thus, housekeeping genes are marginally underrepresented in the GWAS selected genes. It is possible that housekeepers and other structural proteins may reduce the effective genome size for both disease association and practical pharmaceutical intervention. Consistent with this hypothesis, gene transcripts from the Online Mendelian Inheritance in Man (OMIM) database (Hamberger, J. et al. Nucl. Acids Res. 37, D793-796 (2008)) are, like our GWAS selected genes, enriched with therapeutically tractable genes with 259 of 2402 genes associated with Mendelian diseases overlapping with the our set of 991 genes, (p 3.4e-33). However it is possible results from GWAS make it into OMIM potentially introducing an ascertainment bias

Taken together, this analysis shows that GWAS genes are significantly more likely to be theoretically “druggable” or “biopharmable” targets than expected by chance. This observation prompted us to investigate which of these GWAS genes are currently pursued as targets by drugs and for what disease indication.

### Drugs Targeting GWAS Associated Genes

We investigated how many of the 991 GWAS associated genes are targeted by drugs already launched or in development (preclinical and clinical). Pharmaprojects (http://www.pharmaprojects.com), a resource compiling worldwide drug and biopharmaceutical discovery pipeline data, provides a comprehensive list of drug projects and their putative targets.

From the Pharmaprojects database, we identified 1,089 genes (corresponding to 6% of the genome) being pursued as a target by a launched small molecule or biopharmaceutical, a candidate in clinical phase (annotated as Phase I to Phase III, pre-registration or registered) or in preclinical development. Terminated entries in the Pharmaprojects database were not included in our analysis, though these could provide additional examples of repositioning in future analyses. Of the 991 selected GWAS genes, 155 (16%, p<3E-34) had an associated drug project (FIG. 1). This excess was compatible with drug target genes being particularly important in human biology, as even slight variations within these genes (i.e. SNPs) are associated with human diseases/traits. Compared to 1,089 of 19,258 human genes, the GWAS gene set is enriched 2.7-fold in targets pursued by drugs, which is more than expected by chance (15.6% vs 5.7%, p<3.5e-34). This extends the theoretical expectation of GWAS druggability to practical application in real and candidate drug molecules. It is important to note that this analysis included small molecules, biologicals (including protein therapeutics and monoclonal antibodies) but also other modalities such as antisense drugs.

We then compared the disease indications pursued by the drugs with the GWAS trait for each of these 155 genes to identify matches and mismatches in disease/indications. The analysis was done manually because the disease related terms use different vocabularies in the catalogue of GWAS studies and Pharmaprojects (see methods and supplementary information). On closer inspection, a total of 17 additional rows were eliminated where the GWAS traits did not correspond to a disease indication (this was the case for instance for CRP levels). We identified 97 matches between a drug indication and a GWAS trait corresponding to 63 individual genes and 52 GWAS traits (FIG. 1); these observations could be considered as supportive for the particular indication being pursued. In contrast, we also detected 123 mismatches, which included 51 GWAS traits and 92 individual genes (FIG. 1). These mismtach findings could be the basis for drug repositioning opportunities.

### GWAS Studies Supporting a Drug Indication

Table 3 contains 12 selected examples of matches between a GWAS trait and a drug indication for the associated GWAS gene. One of the best known examples of an identical match is the 3-hydroxy-3-methylglutaryl-CoA reductase (HMGCR) gene. The product of HMGCR is targeted by statins, a well known class of cholesterol lowering medications, and SNPs within this gene have been unambiguously associated with LDL cholesterol levels in multiple GWAS studies (Kathiresan, S. et al. Nat. Genet. 40, 189-197 (2008) and (Burkhardt, R. et al. Arterioscler. Thromb. Vasc. Biol. 28, 2078-2084 (2008)). An additional example is the interleukin 12B gene (IL12B). IL12B has been associated through GWAS with autoimmune inflammatory diseases such as Crohn's (Barrett, J. C. et al. Nat. Genet. 40, 955-962 (2008) and psoriasis (Nair, R. P. et al. Nat. Genet. 41, 199-204 (2009) and there is an approved human monoclonal antibody (mAb) Ustekinumab (Centocor/Janssen-Cilag), targeting IL12B, currently marketed for psoriasis and with a Phase II program for Crohn's disease. For less advanced drug development programs a match could provide more confidence for the disease indication. For example, Mellitech is a small Biotech company that has a preclinical program for type 2 diabetes with small molecule agonists of the solute carrier family 30 (zinc transporter) member 8 gene (SLC30A8). Several GWAS studies (Scott, L. G. et al. Science 316, 1341-1345 (2007) and Zeggini, E. et al. Science 316, 1336-1341 (2007)) have associated SLC30A8 with type 2 diabetes providing additional reasons to believe in this target for type 2 diabetes, unless of course this program was initiated based on these GWAS studies in the first place.

Tables 2 and 3 also include examples where the GWAS trait is closely related, but not identical to the drug indication reported in Pharmaprojects for the drug of the same target gene. These imperfect matches may pinpoint the right disease indication for the drug. An example is the alpha interleukin 2 receptor (ILR2A) gene that has been associated with Crohn's disease in GWAS (Franke, A. et al. Nat. Genet. 42, 1118-1125 (2010) (see Table 2 and 3). A monoclonal antibody for ILR2A (Novartis) is currently in Phase II to treat ulcerative colitis. Both Crohn's disease and ulcerative colitis are chronic inflammatory bowel diseases but at the time of the analysis ILR2A was not associated with ulcerative colitis by GWAS. The GWAS association with Crohn's suggests that pursuing that indication in addition to ulcerative colitis could be attractive. Other instances of imperfect matches are for cancer indications. For example the cyclin E1 gene (CCNE1) has been associated with bladder cancer in GWAS (Rohtman, N. et al. Nat. Genet. 42, 978-984 (2010)) and is targeted by drugs in Phases I and II identified in the Pharmaprojects database. This class of molecules have been designed to treat a variety of cancer types; however, none of them has been specifically tested for bladder cancer. GWAS information points here to this particular indication.

### GWAS Suggesting Drug Repositioning Opportunities

Table 4 highlights 12 selected examples of drug repositioning opportunities based on 123 mismatches between a GWAS disease trait and a drug indication (for complete list see Table 1 and 2 as well). For example, denosumab (Prolia® Amgen/GSK) is a marketed drug indicated for the treatment of postmenopausal women with osteoporosis at high risk for fracture. Denosumab targets the gene tumor necrosis factor (ligand) superfamily, member 11 (TNFSF11) also known as RANKL. TNFSF11 has been associated with Crohn's disease by GWAS (Franke, A. et al.) and may potentially play a role in inflammatory bowel disease (Ahscroft et al. Immunity 19, 849-861 (2003) and Moschen et al. Gut 54, 479-487 (2005)). More work is required to understand mechanistically the role of TNFSF11, but it is tempting to speculate that denosumab could be re-purposed with a Crohn's disease indication through additional clinical trials. Another drug in advanced development that is also a potential drug repositioning opportunity is RPI-78M (Nutra Pharma). RPI-78M induces interleukin 27 (IL27) and gamma-interferon, and it is in Phase III testing for Adrenoleukodystrophy and for additional diseases such as multiple sclerosis and herpes virus infection in earlier phases. IL27 has also been associated with inflammatory bowel disease (Imielinski, M. et al. Nat. Genet. 41, 1335-1340 (2009)), including Crohn's disease (Franke, A. et al.) in GWAS. Recently, it has been shown that treatment with IL-27 reduces experimental colitis through the suppression of several inflammatory cytokines including IL-17 (Sasaoka, T. et al. Am. J. Physiol. Gastrointest. Liver Physiol. 300, G568-G576 (2011)). In this situation, human genetic and animal studies converge to support inflammatory bowel disease and Crohn's disease specifically as new indications for RPI-78M. A phase I example is for the antibody Biib-033 from Biogen Idec Inc. targeting the leucine rich repeat and Ig domain containing 1 (LINGO-1) gene. Biib-033 is being developed for patients with multiple sclerosis. Several pieces of evidence support LINGO1 as an attractive target for this indication (Mi, S. et al. Int. J. of Biochem. and Cell. Biol. 40, 1971-1978). Interestingly a GWAS result suggests that LINGO1 may also be an attractive target for essential tremor, a neurological disorder (Stefansson, H. et al. Nat. Genet 41, 277-279 (2009); Clark, L. N. et al. Eur. J. of Hum. Genet. 18, 838-843 (2010); and Thier, S. et al. Mov. Disord. 25, 709-715 (2010)). Essential tremor is most commonly treated, with limited success, by beta blockers, antiepileptics or anticonvulsants and for most severe cases a surgical procedure is sometimes used (Louis, E. D. Lancet Neurol. 4, 100-110 (2005)). Therefore it would seem attractive to consider essential tremor as a new indication in the clinical development plan of Biib-033 or more broadly for LINGO1 inhibitors. A preclinical example is Inhibicor (Heat Biologics), an antibody based biological that blocks the tumor necrosis factor (ligand) superfamily, member 15 gene (TNFSF15) which is being investigated in preclinical research for asthma. TNFSF15 has been associated by GWAS with three related traits: Crohn's disease (Barrett, et al. and Franke, et al.) ulcerative colitis (Anderson, C. A. et al. Nat. Genet. 43, 246-252 (2011), and overall inflammatory bowel disease (Chaudhary, D. and Kasaian, M. Curr. Op. in Inv. Drugs 7, 432-437 (2006)). In addition to the GWAS data, several lines of evidence suggest that TNFSF15 could be an attractive target for these diseases. TNFSF15 may play an important role in a Th1-mediated disease such as Crohn's disease (Bamias, G. et al. J. of Imm. 171, 4868-4874 (2003)) and has also been identified as an important modulator in the development of chronic mucosal inflammation by enhancing T(H)1 and T(H)17 effector functions (Takedatsu, H. et al. Gastroenterology 135, 552-567 (2008). More recently it was shown that up-regulation of TNFSF15 expression can promote mucosal inflammation and gut fibrosis (Shih, D. Q, et al. PloS One 6(1), e16090 (2011). The combination of this information with the GWAS findings and the fact that Inhibicor blocks TNFSF15 suggests that repositioning that drug for Crohn's or ulcerative colitis is worthy of consideration.

The selected examples discussed above, have additional supporting information to support alternative indications for these drugs. Other mismatches in Table 4 have less additional support, but are possibly more novel, and thus could provide unexpected drug repositioning possibilities. Such a mismatch is for the dopamine beta-hydroxylase (DBH) gene. Nepicastat (Hoffman-La Roche) is an inhibitor of DBH in phase II development for cocaine addiction and post-traumatic stress disorder. DBH has also been associated in GWAS with smoking cessation (Tobacco and Genetics Consortium Nat. Genet. 42, 441-447 (2010)). It is thus tempting to speculate that DBH inhibitors may be beneficial for smoking cessation, while acknowledging that the direction of effect is not known yet. Another such example is the interleukin 12A (IL12A) gene. Many drugs are being developed to target IL12A from preclinical to Phase III. Some are monoclonal antibodies such as briakinumab (Abbott), others are gene therapies such as EGEN-001 (Egen) with replacement IL12A. A range of indications are being pursued including psoriasis, Crohn's disease and many cancers. Several GWAS have found an association between IL12A and primary biliary cirrhosis Hirschfield, G. M. et al. N. Eng. J of Med. 360, 2544-2555 (2009); Liu, X. et al. Nat. Genet. 42, 658-660 (2010); and Mells, G. F. et al. Nat. Genet. 42, 329-332 (2010)). In this example the genetics from the GWAS appears to be the only validation of the target for biliary cirrhosis and it is possible that these IL12A drugs could be used to treat primary biliary cirrhosis. These cases of indication mismatch in Table 4, with little additional evidence other than the GWAS, will require more investigation, including experimental work. At the same time they could provide genuinely novel opportunities for drug repositioning.

Not all mismatches will lead to successful drug repositioning opportunities. An illustration comes from the nitric oxide synthase 2, inducible gene (NOS2). A range of inhibitors are under development for various disease indications such as mucositis, rheumatoid arthritis, pain and cerebral ischaemia. Recently, SNPs within NOS2 have been associated in GWAS with psoriasis (Stuart, P. E. et al. Nat Genet. 42, 1000-1004 (2010)), raising the possibility that psoriasis may be an attractive new indication for this class of drugs. This possibility was supported by the observations that skin lesions of psoriatic patients show an increase in nitric oxide production (Ormerod, A. D. et al. Arch. Dermatol. Res. 290, 3-8 (1998)). Contrary to these expectations, at least one small study did not identify any clinical improvement when a topical inhibitor of nitric oxide synthesis was applied in 17 psoriatic subjects (Ormerod, A. D. et al. Br. J. Dermatol. 142, 985-990 (2000)). Another example is for the HMGCR gene, which has been associated with body mass index (BMI) by GWAS (Speliotes, E. K. et al. Nat. Genet. 42, 937-948 (2010)). However no impact on BMI has been reported in large numbers of patients treated with statins (Heart Protection Study Collaborative Group Lancet 360, 7-22 (2002)). Negative studies are rarely definitive, but these two examples highlight some of the limitations of the approach proposed here.

Examples of predicted new indications for certain drug classes are presented in Table 2.

TABLE 2

| | | | | | |
|---|---|---|---|---|---|
| | | | | New predicted | Drug class for new |
| Target | Full name of target | Pharmaceutical | Current indication | Indication | indication |

GWAS Trait could be a new indication

| | | | | | |
|---|---|---|---|---|---|
| DBH | dopamine beta-hydroxylase | Nepicastat | Peripheral vascular disease; | smoking cessation | Inhibitor |
| | (dopamine beta-monooxygenase) | | Post-traumatic stress | | |
| | | | disorder; Cocaine dependence. | | |
| IL2RA | interleukin 2 receptor, alpha | Aldesleukin; mAbs | Cancer | Type 1 Diabetes | IL2/mAb |
| TNFSF15 | tumor necrosis factor (ligand) | Inhibicor | Asthma | Crohn/IBD | Inhbitor |
| | superfamily, member 15 | | | | |
| TNFSF11 | tumor necrosis factor (ligand) | Denosumab | Osteoporosis/bone cancer/ | Crohn's disease | mAb/Antagonists |
| | superfamily, member 11 | | | Cancer | |
| MTNR1 | melatonin receptor 1B | Melatonine | Depression/Clock/Migraine | T2DM | some evidence that |
| | | | | | antagonists have |
| | | | | | been explored |
| IL13 | interleukin 13 | GSK anti-IL13 | Asthma (failed) | Psoriasis | Inhibitors |
| IL-10 | interleukin 10 | avi-2; Eg-10; | RA/Crohn/ | Behcet | Inhibitors/Activators |
| | | Hmpl-011; | | | |
| | | Interleukin-10 | | | |
| LINGO1 | leucine rich repeat and Ig domain | Biib-033 | MS | Essential tremor | Inhibitor |
| | containing 1 | | | | (+mAb in PC) |
| CSF1 | colony stimulating factor 1 | Tg-3003 | Cancer | Paget | Pathway blocker |
| | (macrophage) | | | | |
| | | Valproate | Epilepsy | AD | |
| HDAC | | Vorinostat | Adjuvant BC | AD | |
| CR1 | complement component (3b/4b) | Candida hp; Cdx- | Infection, Candida; renal failure | AD | both directions |
| | receptor 1 | 1135; Eti-204; Eti- | | | |
| | | 211 | | | |
| CLU | clusterin | Ab-16b5; | Cancer | AD | Inhibitors |
| | | Clustirsen | | | |
| ICOSLG | inducible T-cell co-stimulator | Amg-557 | SLE | Crohn/Celiac | |
| | ligand | | | | |
| IL18R | Interleukin 18 receptor | IL18R antag | Psoriasis | Crohn/IBD | Inhibitor |
| IL27 | Interleukin 27 | Rpi-78m | Adrenoleukodystrophy; | Crohn/IBD | Inducer of IL27 |
| | | | Infection, herpes virus, | | expression |
| | | | general; Infection, varicella; | | |
| | | | zoster virus; Multiple sclerosis, | | |
| | | | general Myasthenia gravis | | |
| IL12A | Interleukin 12A | Briakinumab; Cancer | Cancer/Crohn/Psoriasis/Infection | Primary biliairy | Both |
| | | gene therapy, | | cirrhosis | |
| | | intrexon; Egen-001; | | | |
| | | Gx-110; Interleukin- | | | |
| | | 12, neumedicines | | | |
| TERT | telomerase reverse transcriptase | Vaccines | Cancer, fibrosis hepatic, | IPF | Inhibitors; |
| | | (PhIII)/TAT153 (PC) | pulmonary; Anaemia | | Activators |
| | | | aplastic | | |
| IL-2/IL-2R | Interleukin 2/Interleukin2 receptor | Anti-IL-2 MaB | inflammatory diseases | Crohn/IBD/vitiligo | |
| IL2RA | interleukin 2 receptor, alpha | IL2RA mAb | UC | Crohn's disease | |
| IL23R | Interleukin 22 receptor | Apg-2305; Fm-202 | Arthritis, psoriatic; Psoriasis | Crohn/IBD/Behcet | Inhibitor |
| | | | | | (ATX3105); |
| | | | | | allosteric modulator |

GWAS pointing to wrong indication

| | | | | |
|---|---|---|---|---|
| HMGCoAR | | Statins | LDL lowering | BMI |
| NOS2 | nitric oxide synthase 2, inducible | NOS2 inhibitors | Inflammatory | Psoriasis |

GWAS pointing to safety signal (i.e. opposite direction)

| | | | | | |
|---|---|---|---|---|---|
| MUC1 | mucin 1, cell surface associated | Anti-mucin Mab | cancer | Crohn | mAb |
| TCF4 | transcription factor 4 | Cwp-231 | cancer | Fuchs's corneal | |
| | | | | dystrophy | |

Selected examples of GWAS studies supporting drug indications: in each example the GWAS trait is identical (rows 1, 2, 3, 8, 10 and 11) or closely related (rows 4, 5, 6, 7, 9 and 12) to the drug indication. Examples are ranked from most advanced drug (launched) to less advanced (Preclinical). The associated gene between each GWAS and the drug is shown. The drug indication and the phase of development for each drug are derived from the Pharmaprojects database. In many cases more drugs for the gene are listed in the database at different phases. The GWAS references are from the catalog of GWAS studies (http://www.genome.gov/gwasstudies). See Table 3.

TABLE 3

| | Most Advanced | | | | |
|---|---|---|---|---|---|
| | Development | | | | |
| | Phase (for the | | | | |
| Drug Name | indication) | Gene | Drug Indication | GWAS Trait | GWAS Reference |
| Statins | Launched | HMGCR | Hypercholesterolaemia | LDL Cholesterol | 16, 17 |
| Ustekinumab | Approved | IL12B | Psoriasis | Psoriasis | 19 |
| Azimilide | III | KCNQ1 | Ventricular fibrillation | QT interval | 50, 51 |
| Ustekinumab | II | IL12B | Crohn's disease | Crohn's disease | 18 |
| anti-IL2 receptor | II | IL2RA | Ulcerative colitis | Crohn's disease | 22 |
| mAb | | | | | |
| TTP-399 | II | GCK | Type 2 diabetes | Fasting glucose-related | 52, 53 |
| | | | | traits | |
| AMG-785/CDP- | II | SOST | Bone regeneration/ | Bone mineral density | 54 |
| 7851 | | | Osteoporosis | | |
| PRX-302 | II | KLK3 | Prostate cancer | Serum prostate-specific | 55, 56 |
| | | | | antigen levels/ | |
| | | | | Prostate cancer | |
| CYC-202 | I/II | CCNE1 | Various cancers | Bladder cancer | 23 |
| | | | (not Bladder) | | |
| ASP2408 | I | CTLA4 | Rheumatoid arthritis | Rheumatoid arthritis | 57, 58 |
| Znt8 agonists | Preclinical | SLC30A8 | Type 2 diabetes | Type 2 diabetes | 20, 21 |
| TAT-153 | Preclinical | TERT | Pulmonary fibrosis | Idiopathic pulmonary | 59 |
| | | | | fibrosis | |

Selected examples of potential opportunities to reposition a drug for a new disease indication based on the GWAS trait. Examples are ranked from most advanced drug (launched) to less advanced (Preclinical). The associated gene between each GWAS and the drug is shown. The drug indication and the phase of development for each drug are derived from the Pharmaprojects database. In many cases more drugs for the gene are listed in the database at different phases. The GWAS references are from the catalog of GWAS studies (http://www.genome.gov/gwasstudies). For the full list, see table 4 in the supplementary material. See table 4.

TABLE 4

| | Most Advanced | | | | |
|---|---|---|---|---|---|
| | Development Phase | | | | |
| Drug Name | (for the indication) | Gene | Drug Indication | GWAS Trait | GWAS Reference |
| Denosumab/ | Launched/ | TNFSF11 | Osteoporosis/ | Crohn's disease | 22 |
| AMG-162 | registered | | Bone cancer | | |
| RPI-78M | III | IL27 | Adrenoleukodystro | Crohn's | 22, 26 |
| | | | phy | disease/Inflammatory | |
| | | | | bowel disease | |
| Azimilide | III | KCNQ1 | Ventricular | Type 2 diabetes | 60, 61, 62 |
| | | | fibrillation | | |
| Briakinumab/ | III/II | IL12A | Psoriasis/Cancer | Primary biliary cirrhosis | 40, 41, 42 |
| EGEN-001 | | | | | |
| ACN-189/AEB- | II | PRKCQ | Psoriasis/Uveitis | Type 1 diabetes | 63, 64 |
| 071 | | | | | |
| Oxelumab | II | TNFSF4 | Asthma | Systemic lupus | 65 |
| | | | | erythematosus | |
| Nepicastat | II | DBH | Cocaine | Smoking cessation | 44 |
| | | | addiction/post- | | |
| | | | traumatic stress | | |
| | | | disorder | | |
| NOX-A12 | I | CXCL12 | Cancer/Stem cell | Coronary heart disease | 66, 67 |
| | | | mobilization | | |
| Biib-033 | I | LINGO-1 | Multiple sclerosis | Essential Tremor | 35 |
| AMG-557 | I | ICOSLG | Systemic lupus | Crohn's disease/Celiac | 21, 40, 52 |
| | | | erythematosus | disease/Ulcerative | |
| | | | | colitis | |
| Inhibicor | Preclinical | TNFSF15 | Asthma | Crohn's | 21, 25, 39, 40 |
| | | | | disease/Inflammatory | |
| | | | | bowel | |
| | | | | disease/Ulcerative | |
| | | | | colitis | |
| Cwp-231 | Preclinical | TCF4 | Cancer | Fuchs's corneal | 68 |
| | | | | dystrophy | |

### Discussion

In the present analysis, we provide evidence that GWAS studies do not only provide insights into the biology of diseases, but may provide an immediate translational opportunity by pointing to alternative indications for drugs. First, we observed that the set of 991 GWAS associated genes in our analysis are significantly more likely to be amenable to modulation by small molecules or biopharmaceuticals than a random set of genes. That is, GWAS-identified genes offer theoretically greater chances of being “druggable” or “biopharmable” than otherwise random genes. Next, we found that 155 of those genes which are the targets of drugs active in pharmaceutical pipelines, a number which is significantly higher than expected by chance. That is, GWAS identified genes are also practically more likely to be active drug targets. 16% of the GWAS genes are active drug targets, while only 6% of the overall genome is actively targeted by drug projects in Pharmaprojects. We classified these 155 examples of genes targeted by pipeline and marketed drugs into two groups. The first group contains instances of matches between the GWAS trait and the drug indication; these observations provide validation for the approach proposed here. It also includes close matches, i.e. instances where the GWAS trait is closely related, but not identical, to the drug indication; these observations may be used for optimal positioning of the drug. The second group contains instances of mismatches between the GWAS trait, pointing to alternative indications for existing drugs or drugs in. development.

It must be noted that some GWAS positive signals are in gene-rich loci where it could be difficult to identify the driving gene. In these cases additional drug repositioning opportunities would require close scrutiny of each region individually. As an example, the 3p31 GWAS locus shows several Chemokine (C—C motif) receptor genes (CCR1, CCR2, CCRL2, CCR3, CCR5, CCR9) associated with celiac disease (Dubois, P. C. et al. Nat. Genet. 42, 295-302 (2010) and Hunt, K. A. et al. Nat. Genet. 40, 395-402, (2008)). A phase III drug targeting CCR9 has been developed for celiac disease whilst several drugs are targeting CCR1, CCR2, CCR3 and CCR3 but do not have celiac disease as an indication. In these instances, additional work is required to determine which of these genes is causative and, subsequently, which drug has the potential to modify the underlying condition. Our analyses are also fundamentally based on situations where the drug target matches a GWAS-identified locus. However, GWAS may hit the ligand, while drug discovery programs target the receptor, or vice versa, or the direction of effect may differ between the GWAS gene and desired drug action. These and other examples suggest that additional pathway information may be useful to further leverage the interaction of GWAS and ongoing drug development programs.

### Methods

### Selection of GWAS Genes.

GWAS data was downloaded from the NHGRI website (http://www.genome.gov/26525384) on Feb. 14, 2011. There were 4,818 rows of GWAS data in this version. This table included 796 publications. We only considered replicated GWAS's, removing from consideration 2,166 rows where the Replication sample size (column 10) was blank or “NR”. We also excluded 737 GWASs with p-value greater than 1e-7, and rejected an additional 400 rows because the traits as specified in column titled Disease/Trait were considered anthropometric and not relevant for a disease focused analysis. The remaining 1,515 (4818-2166-737-400) rows from 361 publications referred to 1,099 gene names in column titled Reported Gene(s). Of these 991 were recognizable as approved HUGO gene names from Entrez Gene. This set of 991 GWAS genes (GWAS-991) was used for further analysis.

### Small Molecule Druggability and Biopharmability of GWAS Genes.

We combined two sets of proteins that had been annotated as being small molecule druggable (Hopkins, A. L. and Groom C. R. Nat. Rev. Drug Discov. 1, 727-730 (2002) and Russ, A. P. and Lampel, S. Drug. Disc. Today 10, 1607-1610 (2005)) to generate a list of 3,191 genes (16.6% of the genome) that may possibly be considered small molecule druggable. We used a two sided Fischer exact test to determine all p-values to assess which overlaps are significant. Biopharmable proteins were defined as those accessible using an antibody or replaceable by a protein therapy. This is hard to determine precisely, but as a first approximation we defined it as those annotated with either a signal peptide or transmembrane domain in EnsEMBL. All protein coding human genes with HGNC symbols were exported from Biomart (www.biomart.org) along with transmembrane domain and signal domain annotation. 7,411 (38.5%) of the 19,258 EnsEMBL genes with HUGO names were annotated with either a signal peptide or transmembrane domain. We obtained a set of 2,375 housekeepers from Dezsö et al. (Dezsö, Z. et al. BMC Biology 6, 49 (2008)). The OMIM data was downloaded on Jan. 18, 2011 from ftp://ftp.ncbi.nih.gov/repository/OMIM/morbidmap. The Entrez gene identifiers were mapped to HUGO names for analysis using Entrez gene.

### Industry Pipelines Analysis.

Industry pipeline data for a list of all biotech and pharmaceutical projects was taken from PharmaProjects (from Informa Healthcare) as of Nov. 1, 2010. We considered all active projects spanning preclinical to marketed drugs that listed one or more human genes as a known target and had an explicit disease indication. This comprised ˜14,000 projects of which 11,462 listed 1,089 human genes as a potential target. At that stage we also disregarded 17 associations with continuous traits which did not directly represent diseases like waist-hip ratio, C-reactive protein, vertical cup-disc ratio fibrinogen levels, aortic root size or platelet aggregation. Both GWAS and Pharmaprojects use non-standard phenotype and disease indication vocabularies, so we had to manually compare them and make a subjective call as to when a GWAS phenotype was a fairly obvious or related match for a Pharmaprojects disease indication and when it was a mismatch. In this phase we considered all cancer phenotypes to match all cancer indications. This step of determining whether GWAS phenotype matches a disease indication is admittedly subjective and laborius. We did not exclude vaccines provided they listed a human target. The original publications were checked for the selected examples included in Table 3 and in the text to determine if the condition was associated unambiguously with the drug target gene or whether several genes underneath the association locus were in the vicinity, making the association ambiguous.

Note that, like the NHGRI GWAS database, Pharmaprojects is also often updated, so additional inclusions and terminations will have occurred since the time of our database freeze.

### CONCLUSION

One of the long-standing arguments in favor of the GWAS approach and the common disease/common variant hypothesis that underpins it, has been the potential to identify new targets or pathways for therapeutic intervention. However despite the identification of many GWAS associated genes and some new knowledge for some diseases the direct and rapid application of GWAS studies to the benefit of patients remains elusive. We investigated potential opportunities in a drug discovery context. We found that GWAS associated genes were more likely to be druggable than a random set of genes. We also identified a list of drugs that target GWAS genes. In some cases it provided additional support that the drug was used for the right or a related disease indication. In a few cases it also provided some exciting opportunities for drug repositioning or repurposing which is an effective way quickly to discover new efficacious and safe drugs.

Our analyses demonstrate that GWAS have the potential for both direct and indirect identification of disease-validated therapeutic targets. Although the obvious and much heralded use of genetic data for prediction and diagnosis in a personalized medicine context have not materialized, there are immediate applications in the selection of targets which are proven to be druggable, in alignment of treatment indications with appropriate therapies, in identification of new, externally validated, indicates for existing diseases, and in better understanding the pathophysiology of complex human diseases. These data offer direct validation and support for the translational utility of GWAS and also offer real therapeutic opportunities in a very short timescale.

### REFERENCES FOR EXAMPLE 1

1. The Wellcome Trust Case Control Consortium. Nature 447, 661-78 (2007)
2. Hampe et al. Nat. Genet. 39, 207-211 (2007)
3. Zeggini et al. Nat. Genet. 40, 638-645 (2008)
4. Goldstein, D. B. N. Engl. J. Med. 360, 1696-1698 (2009)
5. Hirschorn, J. N. N. Engl. J. Med. 360, 1699-1701 (2009)
6. Freedman, M. L. et al. Nat. Genet. 43, 513-518 (2011)
7. Ashburn, T. T. and Thor, K. B. Nat. Rev. Drug. Discov. 3, 673-683 (2004)
8. Hindorff, L. A. et al. Proc. Natl. Acad. Sci. USA 106, 9362-9367 (2009)
9. Maglott, D. et al. Nucl. Acids Res. 39, D52-D57 (2011)
10. Hopkins, A. L. and Groom C. R. Nat. Rev. Drug Discov. 1, 727-730 (2002)
11. Russ, A. P. and Lampel, S. Drug. Disc. Today 10, 1607-1610 (2005)
12. Seal, R. L. et al. Nucl. Acids Res. 39, D514-D519 (2011)
13. Flicek, P. et al. Nucl. Acids Res. 39, D800-D806 (2011)
14. DezsÖ, Z. et al. BMC Biology 6, 49 (2008)
15. Hamberger, J. et al. Nucl. Acids Res. 37, D793-796 (2008)
16. Kathiresan, S. et al. Nat. Genet. 40, 189-197 (2008)
17. Burkhardt, R. et al. Arterioscler. Thromb. Vasc. Biol. 28, 2078-2084 (2008)
18. Barrett, J. C. et al. Nat. Genet. 40, 955-962 (2008)
19. Nair, R. P. et al. Nat. Genet. 41, 199-204 (2009)
20. Scott, L. G. et al. Science 316, 1341-1345 (2007)
21. Zeggini, E. et al. Science 316, 1336-1341 (2007)
22. Franke, A. et al. Nat. Genet. 42, 1118-1125 (2010)
23. Rohtman, N. et al. Nat. Genet. 42, 978-984 (2010)
24. Ahscroft et al. Immunity 19, 849-861 (2003)
25. Moschen et al. Gut 54, 479-487 (2005)
26. Imielinski, M. et al. Nat. Genet. 41, 1335-1340 (2009)
27. Sasaoka, T. et al. Am. J. Physiol. Gastrointest. Liver Physiol. 300, G568-G576 (2011)
28. Mi, S. et al. Int. J. of Biochem. and Cell. Biol. 40, 1971-1978
29. Stefansson, H. et al. Nat. Genet 41, 277-279 (2009)
30. Clark, L. N. et al. Eur. J. of Hum. Genet. 18, 838-843 (2010)
31. Thier, S. et al. Mov. Disord. 25, 709-715 (2010)
32. Louis, E. D. Lancet Neurol. 4, 100-110 (2005)
33. Kugatashan, S. et al. Nat. Genet. 40, 1211-1215 (2008)
34. Anderson, C. A. et al. Nat. Genet. 43, 246-252 (2011)
35. Chaudhary, D. and Kasaian, M. Curr. Op. in Inv. Drugs 7, 432-437 (2006)
36. Bamias, G. et al. J. of Imm. 171, 4868-4874 (2003)
37. Takedatsu, H. et al. Gastroenterology 135, 552-567 (2008)
38. Shih, D. Q, et al. PloS One 6(1), e16090 (2011)
39. Tobacco and Genetics Consortium Nat. Genet. 42, 441-447 (2010)
40. Hirschfield, G. M. et al. N. Eng. J of Med. 360, 2544-2555 (2009)
41. Liu, X. et al. Nat. Genet. 42, 658-660 (2010)
42. Mells, G. F. et al. Nat. Genet. 42, 329-332 (2010)
43. Stuart, P. E. et al. Nat Genet. 42, 1000-1004 (2010)
44. Ormerod, A. D. et al. Arch. Dermatol. Res. 290, 3-8 (1998)
45. Ormerod, A. D. et al. Br. J. Dermatol. 142, 985-990 (2000)
46. Speliotes, E. K. et al. Nat. Genet. 42, 937-948 (2010)
47. Heart Protection Study Collaborative Group Lancet 360, 7-22 (2002)
48. Dubois, P. C. et al. Nat. Genet. 42, 295-302 (2010)
49. Hunt, K. A. et al. Nat. Genet. 40, 395-402, (2008)
50. Pfeufer, A. et al. Nat. Genet. 41, 407-414 (2009)
51. Newton-Cheh, C. et al. Nat. Genet. 41, 399-406 (2009)
52. Prokopenko, I. et al. Nat. Genet. 41, 77-81 (2008)
53. Dupuis, J. et al. Nat. Genet. 42, 105-116 (2010)
54. Styrkarsdottir, U. et al. Nat. Genet. 41, 15-17 (2009)
55. Gudmundsson, G. et al. Sci. Transl. Med. 15, 62ra92 (2010)
56. Eeles, R. A. et al. Nat. Genet. 40, 316-321 (2008)
57. Gregersen, P. K. et al. Nat. Genet. 41, 820-823 (2009)
58. Stahl, E. A. et al. Nat. Genet. 42, 508-514 (2010)
59. Mushiroda, T. et al. J. Med. Genet. 45, 654-656 (2008)
60. Yasuda, K. et al. Nat. Genet. 40, 1092-1097 (2008)
61. Voight, B. F. et al. Nat. Genet. 42, 579-589 (2010)
62. Tasi, F. G. et al. PloS Genet. 19, e1000847(2010)
63. Barrett, J. C. et al. Nat. Genet. 41, 703-707 (2009)
64. Cooper, J. D. et al. Nat. Genet. 41, 1399-1401 (2008)
65. Han, J. W. et al. Nat. Genet. 41, 1234-1236 (2009)
66. Samani, N. J. et al. N. Eng. J. Med. 357, 443-453 (2007)
67. Schunkert, H. et al. Nat. Genet. 43, 333-338 (2011)
68. Baratz, K. H. et al. N. Eng. J. Med. 363, 1016-1024 (2010)

### Example 2

### Additional Indications for Drug Repositioning

Based on the methods provided herein new therapeutic indications are provided for drugs and biotherapeutics according to Table 1 previously shown. The column designated “New suggested indication” of the Table 1 provides new therapeutic indication determined by the methods of the present invention for the corresponding drugs and classes of therapy listed in the column designated “All drugs” of Table 1. The compounds and/or drugs presented in Table 1 can be used for the treatment of at least one corresponding “New Suggested Indication” in the same row according to Table 1 and/or for the making of a medicament for the treatment of the corresponding “New Suggested Indication.”

1-20. (canceled)

21. A method of treating Crohn's disease in a human in need thereof, comprising administering to said human at least one compound selected from the group consisting of: a T-cell co-stimulator, a T-cell co-stimulatory ligand, an IL-18 receptor antagonist and/or inhibitor, an inducer of IL27 expression, an anti-IL2 receptor mAb, a chemokine (C—C motif) ligand 2 inhibitor, a chemokine (C—C motif) ligand antagonist, an estrogen related receptor alpha binding agent, galactosylceramidase, a monoclonal antibody to anti-Intercellular adhesion molecule 3 (ICAM3), a monoclonal antibody to ICOS, an IL-23 receptor inhibitor, an IL-23 receptor antagonist, a Janus kinase 2 inhibitor, a leucine-rich repeat kinase 2 inhibitor, mucin 1, a cell surface associated inhibitor, a signal transducer and activator of transcription 3 (acute-phase response factor) inhibitor, and a tyrosine kinase 2 inhibitor.

22. The method of claim 21, wherein the human also has Celiac disease, irritable bowel syndrome, and/or inflammatory bowel disease.

23. The method of claim 21, wherein the compound is a T-cell co-stimulator or a T-cell co-stimulatory ligand.

24. The method of claim 23, wherein the compound is a T-cell co-stimulator that is an antibody that binds B7 related proteins.

25. The method of claim 24, wherein said anti-body that binds B7 related proteins is a human antibody that binds to 137 related protein-1 (B7RP-1).

26. The method of claim 21, wherein the compound is a signal transducer and activator of transcription 3 (STAT3) inhibitor.

27. The method of claim 26, wherein said compound is selected from: OPB-31121; OPB-51602; Bardoxolone methyl; Brivudine, and RESprote.

28. A method of treating multiple sclerosis in a human in need thereof, comprising administering a STAT3 inhibitor to said human.

29. A method for repositioning a pharmaceutical, comprising the steps of:
a. selecting at least one target gene, gene product, or loci associated with the treatment of at least one first disease, trait, or phenotype by said pharmaceutical,
b. identifying at least one second disease, trait, or phenotype associated with least one target gene, gene product, or loci of (a) using genome-wide associated studies, and
c. selecting at least one second disease, trait and/or phenotype based on step (b) for treatment with said pharmaceutical.

30. The method of claim 29, further comprising determining expression, overexpress ion, and/or amplification of said first target gene, gene product, or loci in the at least second disease, trait, or phenotype.

31. The method of claim 29, further comprising identifying additional data or experimental support for target gene, gene product, or loci in said second disease, trait, or phenotype.

32. The method of claim 29, wherein step (b) comprises identifying at least one second disease, trait, or phenotype associated with a target gene or loci and the method further comprises identifying at least one SNP in said target gene in said second disease, trait, or phenotype.
